(19) United States
(12) Patent Application Publication
(10) Pub. No.: US 2024/0151709 A1
(43) Pub. Date: May 9, 2024

(54) COMPOSITIONS AND METHODS FOR TRACING CELL NETWORKS

(71) Applicant: The Brigham and Women`s Hospital, Inc., Boston, MA (US)

(72) Inventors: Francisco J. Quintana, Jamaica Plain, MA (US); Michael A. Wheeler, Roslindale, MA (US); Iain C. Clark, Berkeley, CA (US)

(21) Appl. No.: 18/279,778

(22) PCT Filed: Mar. 1, 2022
(86) PCT No.: PCT/US2022/018296

§ 371 Date: Aug. 31, 2023

Related U.S. Application Data
(60) Provisional application No. 63/155,067, filed on Mar. 1, 2021.

(51) Int. Cl. G01N 33/50; C12N 15/86

(52) U.S. Cl. CPC G01N 33/5032; C12N 15/86; G01N 33/5023; G01N 33/5058; C12N 2760/20122; C12N 2760/20143; C12N 2760/20162; G01N 2333/145

(57) ABSTRACT

The present application relates to compositions and methods for tracing cell networks, e.g., for investigation of cell interactions in the CNS with single cell resolution.

### CLAIM OF PRIORITY

This application is the U.S. National Phase Application under 35 U.S.C. § 371 of International Patent Application No. PCT/US2022/018296, filed on Mar. 1, 2022, which claims the benefit of U.S. Provisional Patent Application Ser. No. 63/155,067, filed on Mar. 1, 2021. The entire contents of the foregoing are hereby incorporated by reference.

### SEQUENCE LISTING

The instant application contains a Sequence Listing which has been submitted electronically in ASCII format and is hereby incorporated by reference in its entirety. Said ASCII copy, created on Aug. 30, 2023, is named 29618_0328WO1_SL.txt and is 74,672 bytes in size.

### TECHNICAL FIELD

The present application relates to compositions and methods for tracing cell networks, e.g., for investigation of cell interactions in the CNS, with single cell resolution.

### BACKGROUND

Astrocytes are central nervous system (CNS)-resident glial cells with important roles in health and disease (1-4). Astrocyte functions in development, homeostasis and disease are controlled by their interactions with other cells in the CNS (1, 4-8). For example, astrocyte interactions with microglia regulate synaptic pruning (9), neurodegeneration (8) and CNS inflammation (10). In addition, in the context of autoimmune CNS disorders such as multiple sclerosis (MS) and its pre-clinical model, experimental autoimmune encephalomyelitis (EAE), astrocyte activation is also modulated by T cells and other peripheral immune cells recruited to the inflamed CNS (1, 2, 10-14). However, the full extent of cell interactions that control astrocyte responses and the molecular mechanisms involved are poorly understood. The investigation of those interactions is further complicated by the heterogeneity of astrocytes and other cell types, and the subsequent need to define the specific cell subsets participating in interactions of interest.

High throughput genomic approaches such as single-cell RNA-seq (scRNA-seq) and spatial transcriptomics can profile thousands of individual cells, but challenges remain in applying these approaches to study cell interactions. Moreover, although new techniques can profile immune cell interactions (15, 16) and cell networks based on the sequencing of microdissected units (17) or the use of photoactivatable markers (15, 18, 19), these approaches cannot easily profile cell interactions in the CNS and may fail to detect interactions involving only a small subset of cells.

### SUMMARY

Described herein are methods to identify cell interactions, e.g., in the CNS, and the molecular phenotypes of interacting cells in vivo. The present methods use G-deficient pseudorabies virus (RabΔG), engineered to express a barcode, e.g., a fluorescent mRNA-encoded barcode as it spreads between interacting cells, allowing the reconstruction of cellular interactions in vivo via scRNA-seq. By encoding spatial relationships directly into the transcriptome, the present methods detect cell interactions that otherwise would not be detected by single cell profiling alone. Using one embodiment of these methods, referred to herein as Rabies Barcode Interaction Detection followed by sequencing (RABID-seq), we identified a novel microglia-astrocyte interaction mediated by Sema4D-PlexinB2 signaling, which drives CNS pathology in EAE and potentially in MS. In summary, the present methods provide a new approach for the comprehensive investigation of cell interactions in the CNS with single cell resolution.

Thus, provided herein is a recombinant rabies virus comprising: a polynucleotide encoding a foreign virus envelope protein; and a polynucleotide encoding: a fluorescent protein; a barcode sequence flanked by a first common sequence and a second common sequence; and a 3′ poly(A) tail, wherein the recombinant rabies virus does not encode a functional G protein.

In some embodiments, the foreign virus envelope protein is selected from the group consisting of a retrovirus envelope protein, a paramyxovirus envelope protein, an alphavirus envelope protein, an orthomyxovirus envelope protein, a vesiculovirus envelope protein, and combinations thereof. In some embodiments, the foreign virus envelope protein is avian sarcoma leukosis virus (ASLV) envelope EnvA (ASLV EnvA).

In some embodiments, the fluorescent protein is selected from the group consisting of a green fluorescent protein, a red fluorescent protein, a yellow fluorescent protein, a blue fluorescent protein, a cyan fluorescent protein, an orange fluorescent protein, and combinations thereof. In some embodiments, the fluorescent protein is a red fluorescent protein, optionally mCherry.

In some embodiments, the barcode sequence is about 15 to about 38 nucleotides long.

In some embodiments, the barcode sequence comprises repeated regions. In some embodiments, the barcode sequence comprises repeats of VHDBVHDB (SEQ ID NO:2). In some embodiments, the barcode sequence comprises three repeats of VHDBVHDB (SEQ ID NO:2). In some embodiments, the repeated region(s) are separated by a spacer. In some embodiments, the spacer is an AT dinucleotide. In some embodiments, the barcode sequence comprises VHDBVHDBATVHDBVHDBATVHDBVHDB (SEQ ID NO:1).

Also provided herein is a method for identifying cell-cell contacts in a network of living cells comprising: (i) providing a network of living cells comprising rabies virus-infection-competent cells expressing a receptor for the foreign envelope protein and a functional rabies virus G protein, and rabies virus-infection-incompetent cells that cannot be directly infected by a recombinant rabies viruses described herein (ii) contacting the network of living cells with a recombinant rabies viruses described herein, and maintaining the network under conditions sufficient for the rabies virus to spread from the infection-competent cells to the infection-incompetent cells; and (ii) isolating mRNA transcript(s) from cell(s) expressing the fluorescent protein.

In some embodiments, the method further comprises (iii) attaching a first adapter comprising a first common sequence to the 5′ end and a second adapter comprising a second common sequence to the 3′ end of the mRNA transcript(s); and (iv) sequencing the mRNA transcript(s), thereby generating mRNA transcript sequence(s).

In some embodiments, the foreign envelope protein is avian sarcoma leucosis virus (ASLV) envelope EnvA (ASLV EnvA) and the receptor for the foreign envelope protein is ASLV EnvA receptor TVA.

In some embodiments, isolating mRNA transcript(s) from cell(s) expressing the fluorescent protein comprises fluorescence-activated cell sorting (FACS). In some embodiments, isolating mRNA transcript(s) from cell(s) expressing the fluorescent protein comprises in situ hybridization, capture or capture of the mRNA transcript(s). In some embodiments, isolating mRNA transcript(s) from cell(s) expressing the fluorescent protein comprises fluorescent in situ hybridization (FISH).

In some embodiments, either the first adapter, the second adapter, or both further comprises a cell barcode. In some embodiments, the first adapter, the second adapter, or both further comprises a unique molecular identifier (UMI).

In some embodiments, the method further comprises analyzing the sequences of the mRNA transcript(s) to trace networks within the target cells by: (i) identifying rabies virus barcode sequence(s) amongst the mRNA transcript sequence(s); and (ii) determining which cell(s) of the living network the rabies virus barcode sequence(s) originated from.

In some embodiments, the network of living cells is in a living mammal. In some embodiments, the network of living cells is in the central nervous system of the mammal. In some embodiments, the network of living cells is outside a living mammal. In some embodiments, the network of living cells is a tissue sample from a living mammal. In some embodiments, the tissue sample is a tumor sample. In some embodiments, the tumor is a brain tumor. In some embodiments, the brain tumor is glioblastoma. In some embodiments, the network of living cells is a mammalian cell culture. In some embodiments, the tissue sample is a xenograft. In some embodiments, the network of living cells is an organoid.

In some embodiments, providing a network of living cells comprises transducing a network of living cells with a vector comprising a nucleic acid sequence encoding a cell- or tissue-specific promoter, a nucleic acid sequence encoding a receptor for the foreign envelope protein, and a nucleic acid sequence encoding a functional rabies virus G protein, wherein the network of living cells comprises multiple cell and/or tissue types, and the cell- or tissue-specific promoter is specific is specific for some, but not all, of the cell and/or tissue types.

In some embodiments, the foreign envelope protein is avian sarcoma leucosis virus (ASLV) envelope EnvA (ASLV EnvA) and the receptor for the foreign envelope protein is the ASLV EnvA receptor TVA.

In some embodiments, the cell- or tissue-specific promoter is a CNS cell specific promoter.

In some embodiments, the vector is a lentiviral vector.

Also provided herein are compositions comprising a recombinant rabies virus described herein and a network of living cells comprising rabies virus-infection-competent cells expressing a receptor for the foreign envelope protein and a functional rabies virus G protein, and rabies virus-infection incompetent cells that cannot be directly infected by the recombinant rabies.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1K show reconstruction of single-cell transcriptomes and connectomes using RABID-seq. FIGS. 1A-1D show barcoded RabΔG virus is delivered with intracranial injection, and barcodes transfer to neighboring cells as RabΔG spreads throughout interacting cells (FIG. 1A). The RabΔG genome expresses mCherry, which enables recovery and sequencing of virus-infected cells. The mCherry transcript harbors a unique barcode with semi-random structure, flanked by constant regions to facilitate amplification (FIG. 1B) (FIG. 1B discloses SEQ ID NO: 1). Flow cytometry recovery of mCherry⁺ cells from the CNS (FIG. 1C). Single-cell RNA-sequencing of mCherry⁺ cells (FIG. 1D). FIG. 1E shows fraction of uniquely labeled cells as a function of RabΔG barcode library diversity and number of cells transduced. FIG. 1F shows fraction of the in vivo network captured using inDrop (maximum 60% cell capture rate over a maximum period of 12 hours of encapsulation) as a function of the number of connections that each cell makes for different numbers of transduced cells. FIGS. 1G-1I show RabΔG pseudotyping for cell targeting. Schematic of RabΔG pseudotyping workflow and cell infectability (FIG. 1G). Pseudotyped virus only infects HEK293-TVA cells in vitro, which express the envelope protein of subgroup A (EnvA) receptor, TVA (FIG. 1H). Percent of HEK293 or HEK293-TVA cells infected with pseudotyped RabΔG virus (FIG. 1I). n=4 per group. Unpaired two-tailed t-test. FIG. 1J shows generation of scRNA-seq libraries from inDrop using a SMART-seq approach with template switching and whole transcriptome amplification (WTA). Top-right: WTA material is further amplified using a 2-step approach with mCherry-specific primers followed by PCR primers targeting the constant region flanking the barcode. Bottom-right: Sequencing libraries are prepared from WTA product to produce scRNA-seq libraries. FIG. 1K shows linkage of transcriptome and connectome data enables reconstruction of genome-wide transcriptional signatures of interacting cells in vivo. Data shown as mean±SEM. ***p<0.001. ns: p>0.05.

FIGS. 2A-2D show RABID-seq analysis of astrocyte cell interactions in naive and EAE mice. FIG. 2A shows transgenic mouse line generated to target Gfap-expressing cells with the EnvA-TVA system. FIG. 2B shows EAE disease course. Mice were transduced with barcoded rabies at EAE priming phase and brains were harvested 7.5 days post infection for scRNA-seq. FIG. 2C shows tSNE plots of single-cell RABID-seq data from naive and EAE mice. The number of cells that passed bioinformatic filters is displayed near the origin. FIG. 2D shows chord diagram summaries of astrocyte cell interactions in naive and EAE mice. Percentages are shown relative to the total number of connections. n is the number of cells of each cell type.

FIGS. 3A-3B show analysis of astrocyte—microglia interactions during EAE by RABID-seq. FIG. 3A is a schematic of heterogeneous interactions between astrocytes and microglia during EAE. FIG. 3B shows analysis by GO: molecular function of microglia connected to >90^{th} percentile pro-inflammatory astrocytes.

FIGS. 4A-4B show RABID-seq identifies a novel role for Sema4D-PlexinB2 signaling in microglia-astrocyte communication. FIG. 4A shows differentially regulated axon guidance pathways in astrocyte-microglia networks in EAE vs. naïve mice. FIG. 4B shows differentially regulated axon guidance pathways in microglia connected to astrocytes in EAE vs. naïve mice.

FIGS. 5A-5C show microglia-astrocyte Sema4D-PlexinB2 signaling promotes CNS inflammation in EAE. FIG. 5A shows Sema4d expression determined by qPCR in primary mouse microglia treated with IL-1β/TNF vs. vehicle. n=6 vehicle, n=5 IL-1β/TNF. Kolmogorov-Smirnov t-test. FIG. 5B shows Nos2 and Il1b expression determined by qPCR in primary mouse astrocytes treated with a recombinant Sema4D fragment with agonistic activity. n>8 biologically independent samples per condition. n=9 per group, n=8 for Nos2 vehicle. Kolmogorov-Smirnov t-test per group. FIG. 5C is a schematic depicting microglial Sema4D binding PlexinB2 expressed in astrocytes.

FIGS. 6A-6G show rabies library barcode design, recovery, and analysis pipeline. FIG. 6A shows nucleotide structure of the mCherry barcode. Barcodes were designed using the 28-base sequence VHDBVHDBATVHDBVHDBATVHDBVHDB (SEQ ID NO:1) that contained three variable stretches encoded by the degenerate bases (IUPAC nucleotide code): V is not T; H is not G; D is not C; B is not A; and two AT anchors. Bioinformatic recovery of the barcode from sequencing reads identified the presence of conserved 5′ or 3′ PCR handles (18 bp each), extracted the intervening 28 bases, filtered sequences with the correct VHDBVHDB (SEQ ID NO:2) structure between AT anchors, and error corrected barcodes using a Levenshtein distance of one. FIG. 6A discloses SEQ ID NOS 1 and 83-85, respectively, in order of appearance. FIG. 6B shows: amplification of barcodes from plasmid libraries used primers that target conserved handles flanking the barcode, generating a sequencing-ready Illumina library. FIG. 6C shows number of unique barcodes recovered after bioinformatic processing versus sub-sampled read depth showed sequencing saturation and an approximate plasmid library barcode diversity of 1.5 million unique sequences. FIG. 6D shows after viral packaging and EnvA pseudotyping, the diversity of the viral library was estimated at approximately 10,000-100,000 unique sequences. FIG. 6E shows molecular strategy for recovering RabΔG barcodes from amplified cDNA. Step one: limited cycle (˜5) and qPCR quantification (N cycles) was followed by a semi-nested 10-cycle PCR primed off one of the flanking handles. A representative Bioanalyzer trace from a sequencing-ready library is shown. FIG. 6F shows cells containing both transcriptome and RabΔG barcode information were retained for analysis. FIG. 6G shows networks were generated and trimmed based on UMI counts of shared barcodes to create final networks. Vertices represent cells, edges represent shared rabies barcodes.

FIG. 7 is a schematic of RabΔG barcoding of glia. EnvA pseudotyped rabies virus infects TVA-expressing astrocytes. G-expressing astrocytes produce functional rabies virus that infects neighboring cells, but cannot be spread by glycoprotein G-deficient connected cells.

FIG. 8A shows an example of an organotypic culture procedure for RABID-seq. In this example, specimens are cut into 350 μm slices and placed on 0.4 μm membrane inserts, infected with lentivirus expressing the TVA envelope receptor and rabies glycoprotein (“G”), and 2 days later, barcoded, pseudotyped, glycoprotein G deficient rabies virus is added. Samples are then processed, cDNA generated, and mCherry barcodes amplified using mCherry and Indrop forward primers, followed by handle and index primers. A 285 bp barcode product is detected by Bioanalyzer.

FIG. 8B shows cell-cell interactions detected in the tumor microenvironment of human glioblastoma patient samples (n=3).

FIG. 9A shows a map of plasmid pLenti-EF1a-G-2A-TVA-2A-EGFP.

FIG. 9B shows a map of plasmid pLenti-Gfap-G-2A-TVA-2A-EGFP.

FIG. 9C shows a map of plasmid pLenti-Prom-TVA-2A-G-2A-EGFP.

### DETAILED DESCRIPTION

Powerful techniques have been developed for inferring cell interactions based on spatial transcriptomics (42-48), physical interactions (15, 16), or co-localization in tissue (17, 18). However, these approaches remain difficult to apply for the study of cells in the CNS due to technical complexity, throughput limitations, or a lack of single-cell resolution with respect to transcriptional or interaction information. To overcome these limitations, we developed unbiased, high-throughput and accessible methods for the study of cell interactions, one embodiment of which is referred to herein as RABID-seq. The present methods use G-deficient pseudotyped rabies virus (RabΔG) engineered to express a unique detectably labeled mRNA barcode that is polyadenylated and captured in RNA-sequencing experiments, enabling the identification of cell interactions directly from transcriptomic data, thereby exploiting the maturity and ubiquity of next generation sequencing (NGS) methods (49) while lowering the barrier to technology adoption. The use of TVA-expressing cells (e.g., transgenic animals or other engineered cells such as cell lines lines, tissue samples, or organoids) and rabies glycoprotein-G in target cells of interest allows the present methods to be used for the study of cell interactions, e.g., outside the CNS.

The present methods are scalable to millions of cells and can be applied to sparse networks and transient interactions, using flow cytometry to recover fluorescently labeled barcoded cells. Additional antibody-based sorting can be employed to study interactions involving rare or specific cell subsets. Because these methods read out cell interactions at the sequencing step, it is agnostic to scRNA-seq technology used; any method that captures and reads the labeled mRNA barcode can be utilized for RABID-seq studies, e.g., scRNA-seq platforms such as the 10× Genomics v3 Chromium platform. Moreover, multi-omic approaches that measure RNA and DNA (50), RNA and chromatin (51), or RNA and protein (18), could be combined with the present methods to identify cell interactions and associate them with the regulation of cellular responses at the genomic, epigenetic, and proteomic level. This is an important point considering recent reports on the effects of cell interactions on the epigenetic control of astrocytes and microglia, and the potential effects that epigenetic modifications may have on the long-term responses and disease-promoting activities of CNS-resident cells (1, 52-54).

Secreted factors involved in microglia-astrocyte communication have been identified, with important roles in neurologic disorders (8-11, 55-57). The present methods identified axon guidance molecules as novel participants in astrocyte-microglia interactions. Axon guidance molecules are known to be co-opted in the context of cancer and inflammation (58, 59), but their participation in microglia-astrocyte interactions during CNS inflammation was unknown. Indeed, Sema4D is known to participate in neurodevelopment (60, 61), T-cell activation and T-cell driven microglia activation in EAE (35, 62, 63), but its role in microglia-astrocyte communication has not been previously reported. The present methods identified a novel role for Sema4D-PlexinB1/2 interactions in the microglial control of astrocytes during EAE, and analysis of the scRNA-seq datasets supports a role for SEMA4D-PLEXINB1/2 signaling in the microglial regulation of astrocyte responses during MS. Collectively, these findings suggest that MS therapies under development targeting SEMA4D could be improved if the therapeutic agent reaches the CNS (64). In addition, the present methods showed that PlexinB1/2 signaling in astrocytes mediates their regulation by microglial Sema4D. Plexins interact with neuropilins to control downstream signaling pathways, and neuropilins also participate in signaling pathways activated by VEGF-B, which promotes astrocyte pathogenic responses in EAE and MS (10, 35, 65). Thus, based on their roles in coordinating astrocyte responses to multiple pro-inflammatory stimuli, PlexinB1/2 may provide additional targets for the therapeutic modulation of disease-promoting astrocyte responses in MS and other neurologic disorders.

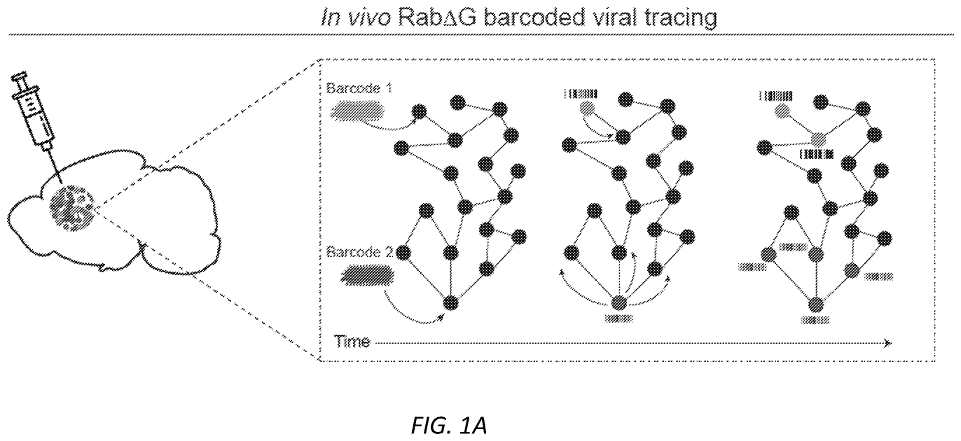

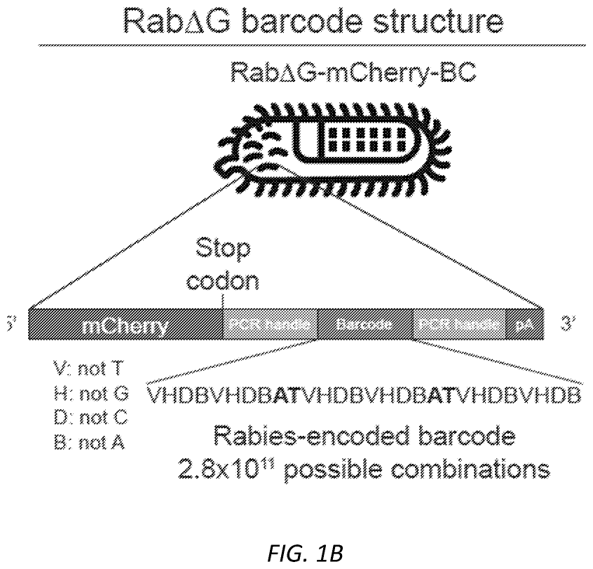

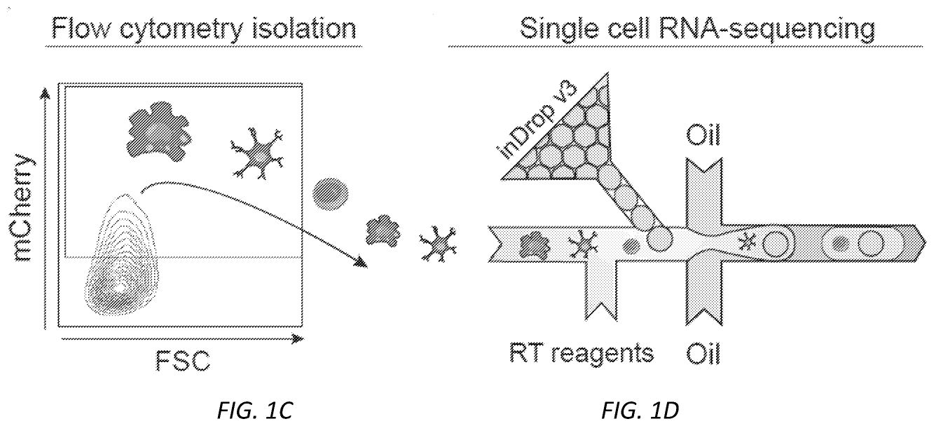

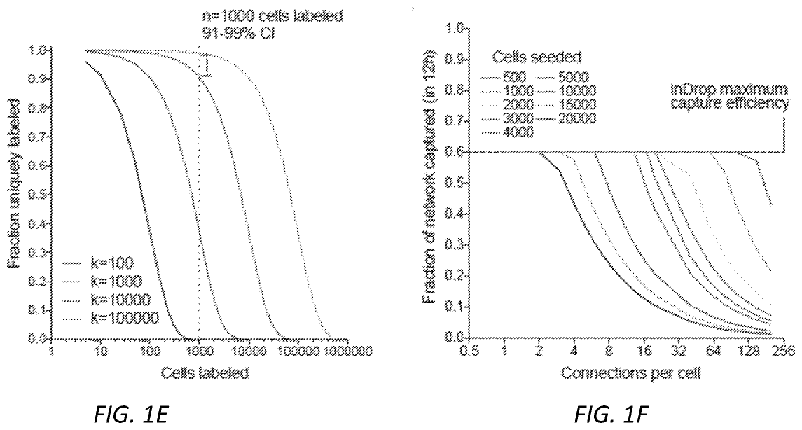

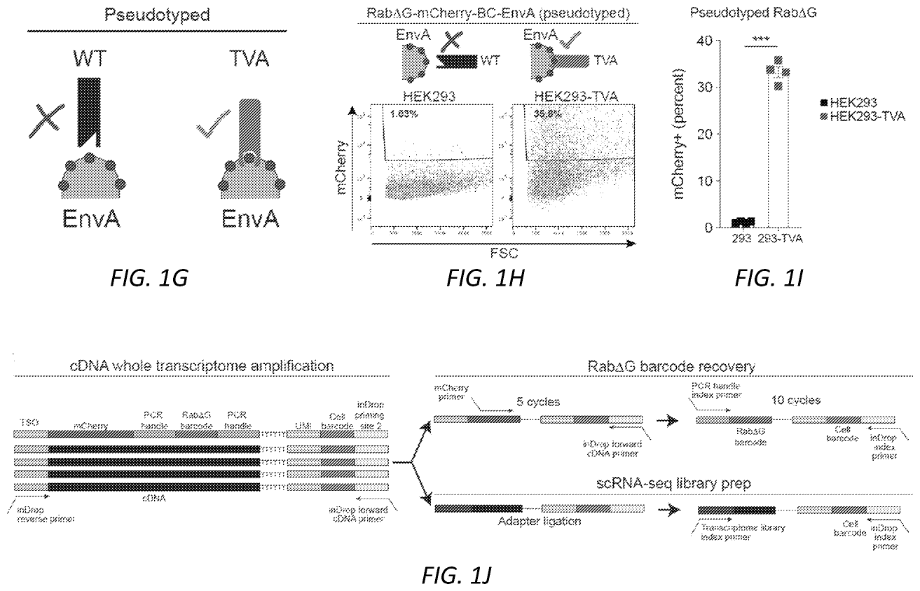

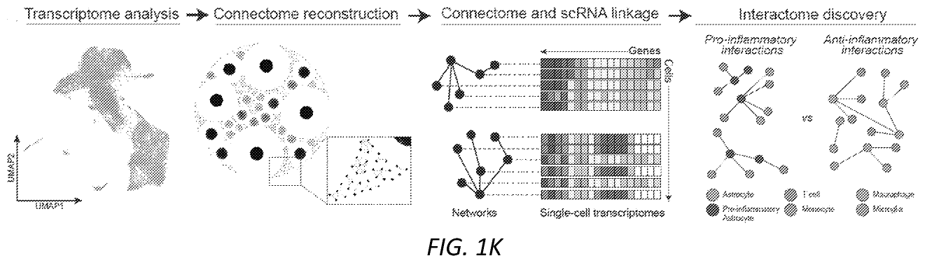

RabΔG is a powerful tool for studying cell interactions because it can be targeted to specific cell types including astrocytes and other glia (20-24) (FIG. 1A). To study astrocyte cell interactions, we engineered the RabΔG virus to express a barcoded reporter gene; in some embodiments, mCherry was used (RabΔG-mCherry-BC), but other fluorescent reporter genes, e.g., as described herein can also be used as well as recombinases (such as Cre recombinase or FLP); Cre-dependent (or independent) actuators of cellular activity such as channelrhodopsin-2 or DREADDs like hM3Dq; methods of ablation including the diphtheria toxin receptor; single guide RNAs utilized by CRISPR/Cas9 or other Cas enzymes to perturb or modify gene function; intersectional targeting strategies such as split Cre or split GFP; and membrane protein(s) (e.g., Thy1) for sorting, e.g., using magnetic beads. Since barcode sequences are inserted prior to the transcriptional stop of the polyadenylated mCherry transcript, the transcribed mRNA barcode can be analyzed by scRNA-seq (FIG. 1B). mCherry expression allows the isolation by flow cytometry of fluorescently-labeled barcoded cells in a RabΔG-transduced cell network (FIG. 1C), enabling the simultaneous analysis of cell transcriptomes and RabΔG barcodes by high throughput droplet-based scRNA-seq (FIG. 1D).

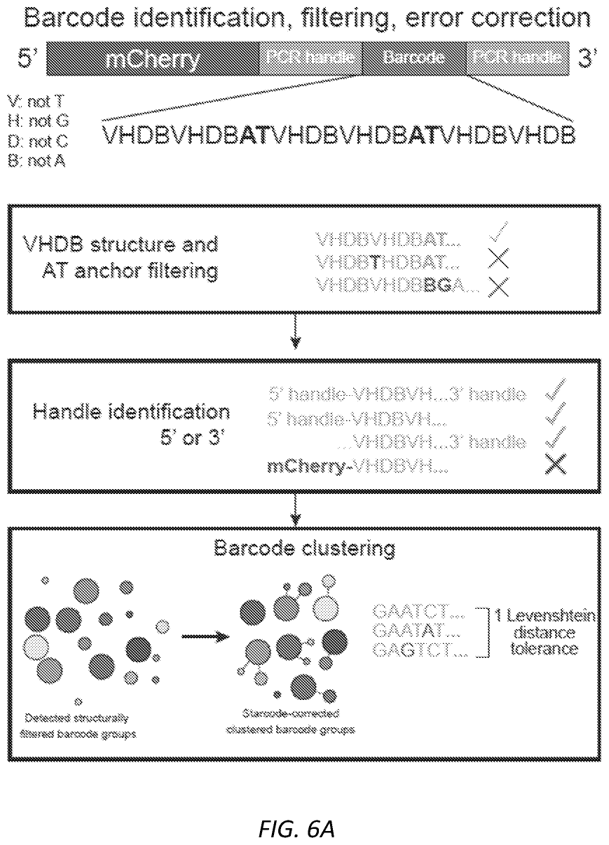

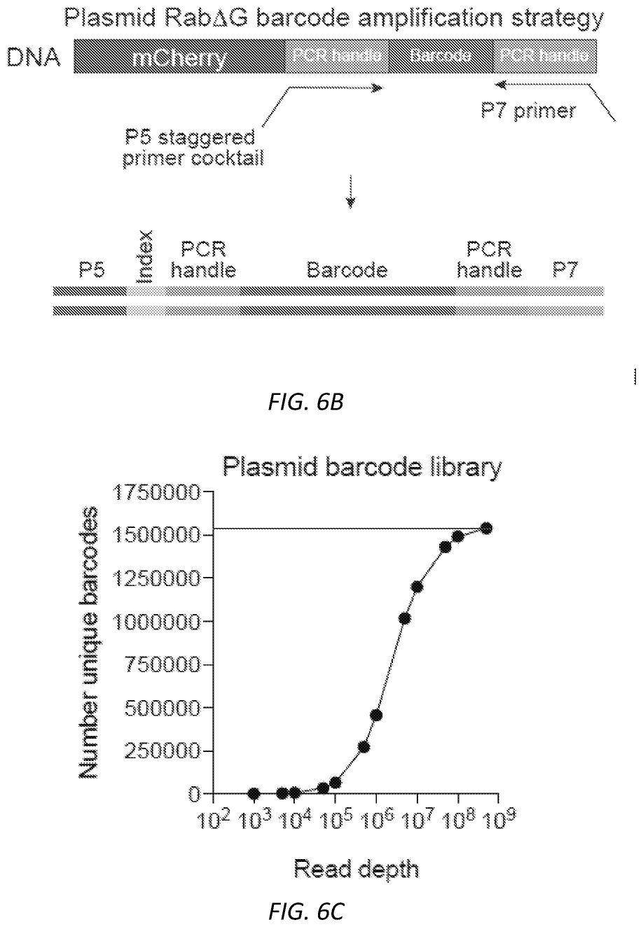

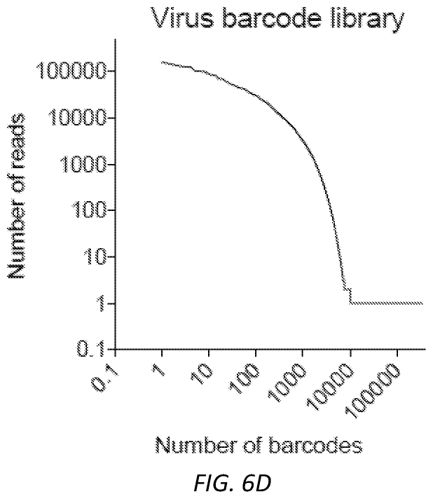

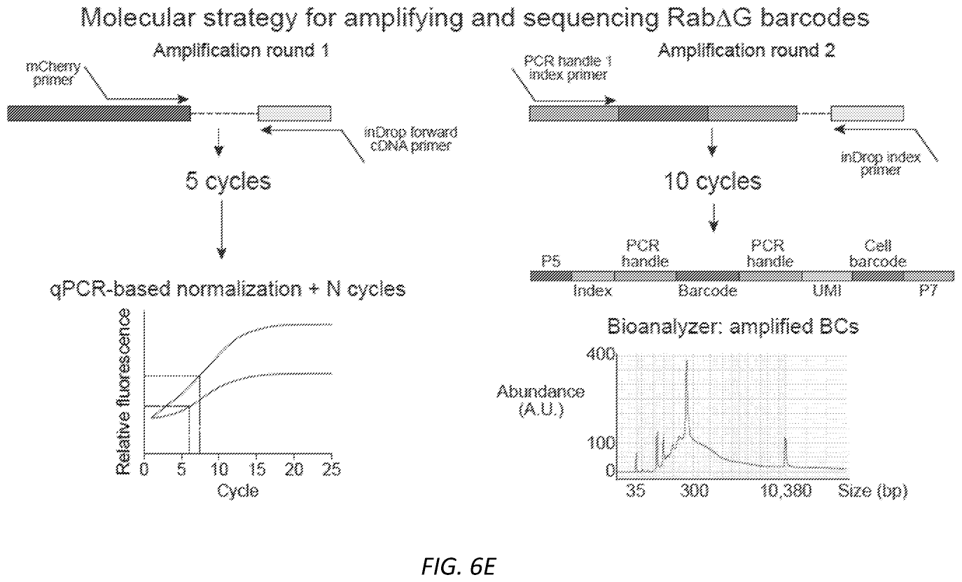

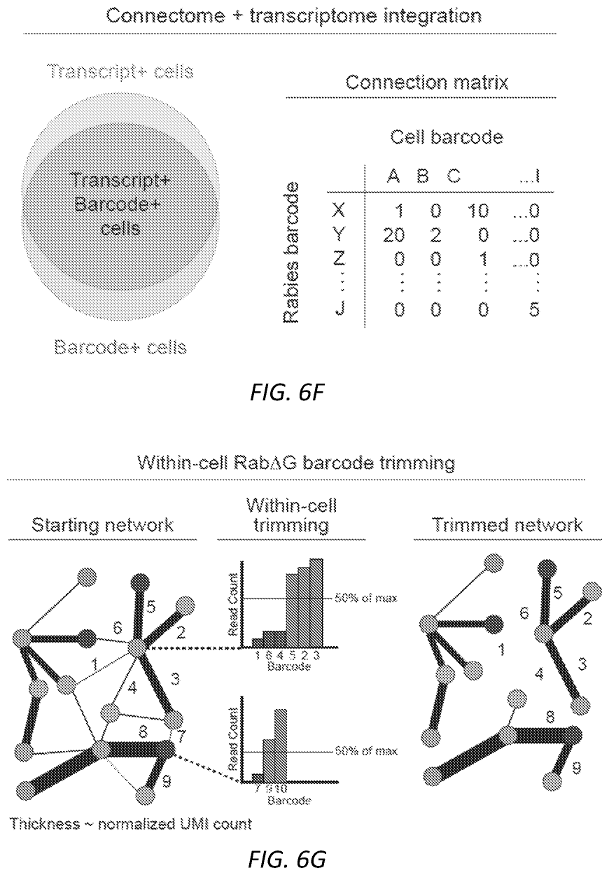

Following amplification and sequencing, we detected approximately 1.5 million unique sequences in the barcoded RabΔG-mCherry-BC plasmid library (FIGS. 6A-6C). We pseudotyped the rabies virus from the barcoded RabΔG-mCherry-BC plasmid library using envelope protein of subgroup A (EnvA)-packaging, which only infects cells expressing the EnvA receptor TVA, thereby allowing the genetic targeting of cells of interest in vivo (20, 25, 26). Since the resulting pseudotyped rabies virus library was estimated to contain 10⁴-10⁵ unique barcodes (FIG. 6D), we predicted that 91%-99% of infected cells will be uniquely barcoded if 1,000 cells are initially infected with the pseudotyped RabΔG-mCherry-BC virus library (FIGS. 1E-1F).

We used an in vitro system to confirm that infection with pseudotyped RabΔG-mCherry-BC virus was restricted to TVA-expressing cells (FIGS. 1G-1I), and developed a PCR-based strategy for amplifying rabies connection barcodes from cDNA generated by the inDrop workflow (FIGS. 1J, 6E). Importantly, RabΔG-mCherry-BC sequencing libraries retained three crucial pieces of information: 1) A scRNA-seq cell barcode to assign RabΔG rabies barcodes to single-cell transcriptome data, 2) A unique barcode structure that allows efficient error correction, and 3) A unique molecular identifier (UMI) to count RabΔG barcode transcripts (FIG. 1J). After sequencing, barcodes were identified, counted, and associated with individual cells captured by scRNA-seq. Interactions between cells were determined by the presence of shared barcodes (FIGS. 6F-6G), allowing the reconstruction of cellular networks with genome-wide transcriptional information in vivo at single-cell resolution (FIG. 1K). Thus, RABID-seq is a novel method for the high throughput analysis.

In summary, we developed methods for the high throughput identification of cell interactions and the molecular phenotype of interacting cells. These methods enable the identification of novel microglia-astrocyte interactions and potential targets for their therapeutic modulation in neurologic diseases. These methods provide useful tools for the comprehensive investigation of CNS cell interactions and their physiologic roles in development, homeostasis and neurologic disorders.

### Recombinant Rabies Virus (RABV)-Based Cell Interaction Tracers

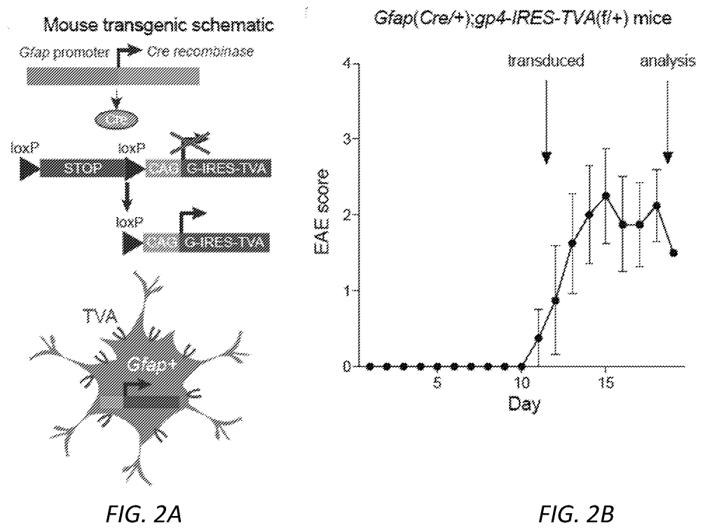

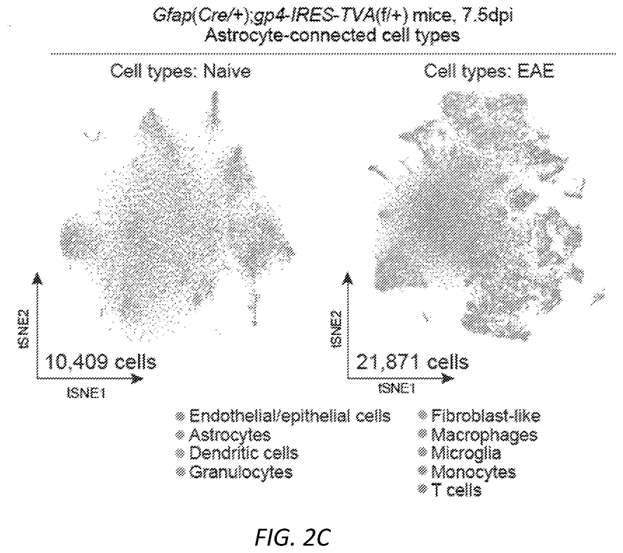

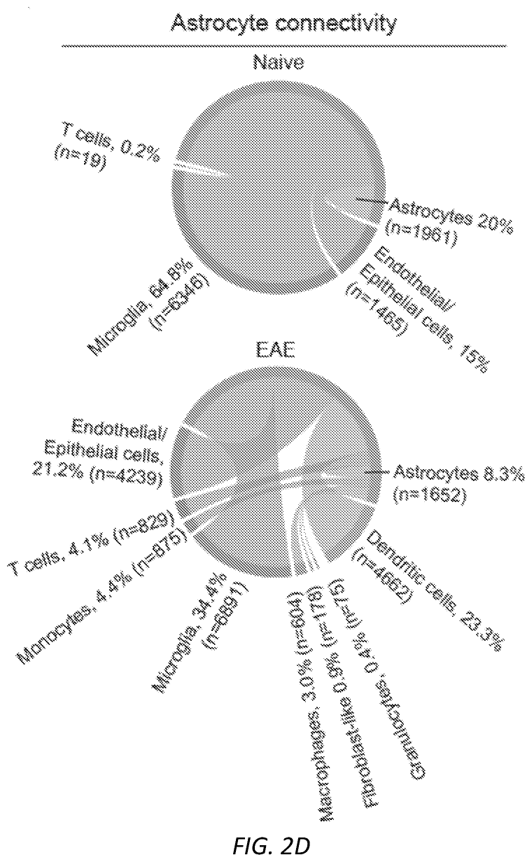

The systems and methods described herein can include the use of barcoded viral tracers. Each barcode is encoded on a fluorescent reporter, enabling the identification of transduced cells by fluorescence activated cell sorting (FACS) (FIG. 2C). The viruses are engineered to infect only genetically defined cells expressing the rabies virus receptor TVA, and to limit spread of the virus only by direct contact from infected cells expressing the rabies glycoprotein G. The recombinant rabies viruses described herein are useful, e.g., in methods for tracing cell networks.

In some embodiments, the viral tracer is a recombinant rabies virus (RABV).

### Rabies Virus Proteins

The rabies virus genome is a single-stranded, antisense, nonsegmented, RNA of approximately 12 kb. There is a leader-sequence of approximately 50 nucleotides, followed by nucleoprotein (N), phosphoprotein (P), matrix protein (M), glycoprotein (G), and polymerase (L) genes. See, e.g., Tordo et al., “The Rabies Virus Genome: an Overview,” Onderstepoort Journal of Veterinary Research, 60:263-9 (1993) and Tordo et al., “Completion of the Rabies Virus Genome Sequence Determination: Highly Conserved Domains among the L (polymerase) proteins of unsegmented negative-strand RNA Viruses,” Virology 165(2):565-76 (1988).

Exemplary rabies virus protein sequences for strain SAD B19 are as follows, though any rabies virus, including orthologs, homologs, mutants, and variants of SAD B19, is suitable for use as a viral tracer.

Rabies virus SAD B19 complete genome (GenBank Accession No. M31046.1; SEQ ID NO:73):

ACGCTTAACAACCAGATCAAAGAAAAAACAGACATTGTCAATTGC
AAAGCAAAAATGTAACACCCCTACAATGGATGCCGACAAGATTGT
ATTCAAAGTCAATAATCAGGTGGTCTCTTTGAAGCCTGAGATTAT
CGTGGATCAATATGAGTACAAGTACCCTGCCATCAAAGATTTGAA
AAAGCCCTGTATAACCCTAGGAAAGGCTCCCGATTTAAATAAAGC
ATACAAGTCAGTTTTGTCAGGCATGAGCGCCGCCAAACTTAATCC
TGACGATGTATGTTCCTATTTGGCAGCGGCAATGCAGTTTTTTGA
GGGGACATGTCCGGAAGACTGGACCAGCTATGGAATTGTGATTGC
ACGAAAAGGAGATAAGATCACCCCAGGTTCTCTGGTGGAGATAAA
ACGTACTGATGTAGAAGGGAATTGGGCTCTGACAGGAGGCATGGA
ACTGACAAGAGACCCCACTGTCCCTGAGCATGCGTCCTTAGTCGG
TCTTCTCTTGAGTCTGTATAGGTTGAGCAAAATATCCGGGCAAAA
CACTGGTAACTATAAGACAAACATTGCAGACAGGATAGAGCAGAT
TTTTGAGACAGCCCCTTTTGTTAAAATCGTGGAACACCATACTCT
AATGACAACTCACAAAATGTGTGCTAATTGGAGTACTATACCAAA
CTTCAGATTTTTGGCCGGAACCTATGACATGTTTTTCTCCCGGAT
TGAGCATCTATATTCAGCAATCAGAGTGGGCACAGTTGTCACTGC
TTATGAAGACTGTTCAGGACTGGTATCATTTACTGGGTTCATAAA
ACAAATCAATCTCACCGCTAGAGAGGCAATACTATATTTCTTCCA
CAAGAACTTTGAGGAAGAGATAAGAAGAATGTTTGAGCCAGGGCA
GGAGACAGCTGTTCCTCACTCTTATTTCATCCACTTCCGTTCACT
AGGCTTGAGTGGGAAATCTCCTTATTCATCAAATGCTGTTGGTCA
CGTGTTCAATCTCATTCACTTTGTAGGATGCTATATGGGTCAAGT
CAGATCCCTAAATGCAACGGTTATTGCTGCATGTGCTCCTCATGA
AATGTCTGTTCTAGGGGGCTATCTGGGAGAGGAATTCTTCGGGAA
AGGGACATTTGAAAGAAGATTCTTCAGAGATGAGAAAGAACTTCA
AGAATACGAGGCGGCTGAACTGACAAAGACTGACGTAGCACTGGC
AGATGATGGAACTGTCAACTCTGACGACGAGGACTACTTTTCAGG
TGAAACCAGAAGTCCGGAGGCTGTTTATACTCGAATCATGATGAA
TGGAGGTCGACTAAAGAGATCTCACATACGGAGATATGTCTCAGT
CAGTTCCAATCATCAAGCCCGTCCAAACTCATTCGCCGAGTTTCT
AAACAAGACATATTCGAGTGACTCATAAGAAGTTGAATAACAAAA
TGCCGGAAATCTACGGATTGTGTATATCCATCATGAAAAAAACTA
ACACCCCTCCTTTCGAACCATCCCAAACATGAGCAAGATCTTTGT
CAATCCTAGTGCTATTAGAGCCGGTCTGGCCGATCTTGAGATGGC
TGAAGAAACTGTTGATCTGATCAATAGAAATATCGAAGACAATCA
GGCTCATCTCCAAGGGGAACCCATAGAGGTGGACAATCTCCCTGA
GGATATGGGGCGACTTCACCTGGATGATGGAAAATCGCCCAACCA
TGGTGAGATAGCCAAGGTGGGAGAAGGCAAGTATCGAGAGGACTT
TCAGATGGATGAAGGAGAGGATCCTAGCTTCCTGTTCCAGTCATA
CCTGGAAAATGTTGGAGTCCAAATAGTCAGACAAATGAGGTCAGG
AGAGAGATTTCTCAAGATATGGTCACAGACCGTAGAAGAGATTAT
ATCCTATGTCGCGGTCAACTTTCCCAACCCTCCAGGAAAGTCTTC
AGAGGATAAATCAACCCAGACTACTGGCCGAGAGCTCAAGAAGGA
GACAACACCCACTCCTTCTCAGAGAGAAAGCCAATCATCGAAAGC
CAGGATGGCGGCTCAAATTGCTTCTGGCCCTCCAGCCCTTGAATG
GTCGGCTACCAATGAAGAGGATGATCTATCAGTGGAGGCTGAGAT
CGCTCACCAGATTGCAGAAAGTTTCTCCAAAAAATATAAGTTTCC
CTCTCGATCCTCAGGGATACTCTTGTATAATTTTGAGCAATTGAA
AATGAACCTTGATGATATAGTTAAAGAGGCAAAAAATGTACCAGG
TGTGACCCGTTTAGCCCATGACGGGTCCAAACTCCCCCTAAGATG
TGTACTGGGATGGGTCGCTTTGGCCAACTCTAAGAAATTCCAGTT
GTTAGTCGAATCCGACAAGCTGAGTAAAATCATGCAAGATGACTT
GAATCGCTATACATCTTGCTAACCGAACCTCTCCCCTCAGTCCCT
CTAGACAATAAAATCCGAGATGTCCCAAAGTCAACATGAAAAAAA
CAGGCAACACCACTGATAAAATGAACCTCCTACGTAAGATAGTGA
AAAACCGCAGGGACGAGGACACTCAAAAATCCTCTCCCGCGTCAG
CCCCTCTGGATGACGATGACTTGTGGCTTCCACCCCCTGAATACG
TCCCGCTGAAAGAACTTACAGGCAAGAAGAACATGAGGAACTTTT
GTATCAACGGAAGGGTTAAAGTGTGTAGCCCGAATGGTTACTCGT
TCAGGATCCTGCGGCACATTCTGAAATCATTCGACGAGATATATT
CTGGGAATCATAGGATGATCGGGTTAGTCAAAGTGGTTATTGGAC
TGGCTTTGTCAGGATCTCCAGTCCCTGAGGGCCTGAACTGGGTAT
ACAAATTGAGGAGAACCTTTATCTTCCAGTGGGCTGATTCCAGGG
GCCCTCTTGAAGGGGAGGAGTTGGAATACTCTCAGGAGATCACTT
GGGATGATGATACTGAGTTCGTCGGATTGCAAATAAGAGTGATTG
CAAAACAGTGTCATATCCAGGGCAGAGTCTGGTGTATCAACATGA
ACCCGAGAGCATGTCAACTATGGTCTGACATGTCTCTTCAGACAC
AAAGGTCCGAAGAGGACAAAGATTCCTCTCTGCTTCTAGAATAAT
CAGATTATATCCCGCAAATTTATCACTTGTTTACCTCTGGAGGAG
AGAACATATGGGCTCAACTCCAACCCTTGGGAGCAATATAACAAA
AAACATGTTATGGTGCCATTAAACCGCTGCATTTCATCAAAGTCA
AGTTGATTACCTTTACATTTTGATCCTCTTGGATGTGAAAAAAAC
TATTAACATCCCTCAAAAGACTCAAGGAAAGATGGTTCCTCAGGC
TCTCCTGTTTGTACCCCTTCTGGTTTTTCCATTGTGTTTTGGGAA
ATTCCCTATTTACACGATACCAGACAAGCTTGGTCCCTGGAGTCC
GATTGACATACATCACCTCAGCTGCCCAAACAATTTGGTAGTGGA
GGACGAAGGATGCACCAACCTGTCAGGGTTCTCCTACATGGAACT
TAAAGTTGGATACATCTTAGCCATAAAAGTGAACGGGTTCACTTG
CACAGGCGTTGTGACGGAGGCTGAAACCTACACTAACTTCGTTGG
TTATGTCACAACCACGTTCAAAAGAAAGCATTTCCGCCCAACACC
AGATGCATGTAGAGCCGCGTACAACTGGAAGATGGCCGGTGACCC
CAGATATGAAGAGTCTCTACACAATCCGTACCCTGACTACCGCTG
GCTTCGAACTGTAAAAACCACCAAGGAGTCTCTCGTTATCATATC
TCCAAGTGTGGCAGATTTGGACCCATATGACAGATCCCTTCACTC
GAGGGTCTTCCCTAGCGGGAAGTGCTCAGGAGTAGCGGTGTCTTC
TACCTACTGCTCCACTAACCACGATTACACCATTTGGATGCCCGA
GAATCCGAGACTAGGGATGTCTTGTGACATTTTTACCAATAGTAG
AGGGAAGAGAGCATCCAAAGGGAGTGAGACTTGCGGCTTTGTAGA
TGAAAGAGGCCTATATAAGTCTTTAAAAGGAGCATGCAAACTCAA
GTTATGTGGAGTTCTAGGACTTAGACTTATGGATGGAACATGGGT
CTCGATGCAAACATCAAATGAAACCAAATGGTGCCCTCCCGATAA
GTTGGTGAACCTGCACGACTTTCGCTCAGACGAAATTGAGCACCT
TGTTGTAGAGGAGTTGGTCAGGAAGAGAGAGGAGTGTCTGGATGC
ACTAGAGTCCATCATGACAACCAAGTCAGTGAGTTTCAGACGTCT
CAGTCATTTAAGAAAACTTGTCCCTGGGTTTGGAAAAGCATATAC
CATATTCAACAAGACCTTGATGGAAGCCGATGCTCACTACAAGTC
AGTCAGAACTTGGAATGAGATCCTCCCTTCAAAAGGGTGTTTAAG
AGTTGGGGGGAGGTGTCATCCTCATGTGAACGGGGTGTTTTTCAA
TGGTATAATATTAGGACCTGACGGCAATGTCTTAATCCCAGAGAT
GCAATCATCCCTCCTCCAGCAACATATGGAGTTGTTGGAATCCTC
GGTTATCCCCCTTGTGCACCCCCTGGCAGACCCGTCTACCGTTTT
CAAGGACGGTGACGAGGCTGAGGATTTTGTTGAAGTTCACCTTCC
CGATGTGCACAATCAGGTCTCAGGAGTTGACTTGGGTCTCCCGAA
CTGGGGGAAGTATGTATTACTGAGTGCAGGGGCCCTGACTGCCTT
GATGTTGATAATTTTCCTGATGACATGTTGTAGAAGAGTCAATCG
ATCAGAACCTACGCAACACAATCTCAGAGGGACAGGGAGGGAGGT
GTCAGTCACTCCCCAAAGCGGGAAGATCATATCTTCATGGGAATC
ACACAAGAGTGGGGGTGAGACCAGACTGTAAGGACTGGCCGTCCT
TTCAACGATCCAAGTCCTGAAGATCACCTCCCCTTGGGGGGTTCT
TTTTGAAAAACCTGGGTTCAATAGTCCTCCTTGAACTCCATGCAA
CTGGGTAGATTCAAGAGTCATGAGATTTTCATTAATCCTCTCAGT
TGATCAAGCAAGATCATGTCGATTCTCATAATAGGGGAGATCTTC
TAGCAGTTTCAGTGACTAACGGTACTTTCATTCTCCAGGAACTGA
CACCAACAGTTGTAGACAAACCACGGGGTGTCTCGGGTGACTCTG
TGCTTGGGCACAGACAAAGGTCATGGTGTGTTCCATGATAGCGGA
CTCAGGATGAGTTAATTGAGAGAGGCAGTCTTCCTCCCGTGAAGG
ACATAAGCAGTAGCTCACAATCATCTCGCGTCTCAGCAAAGTGTG
CATAATTATAAAGTGCTGGGTCATCTAAGCTTTTCAGTCGAGAAA
AAAACATTAGATCAGAAGAACAACTGGCAACACTTCTCAACCTGA
GACTTACTTCAAGATGCTCGATCCTGGAGAGGTCTATGATGACCC
TATTGACCCAATCGAGTTAGAGGCTGAACCCAGAGGAACCCCCAT
TGTCCCCAACATCTTGAGGAACTCTGACTACAATCTCAACTCTCC
TTTGATAGAAGATCCTGCTAGACTAATGTTAGAATGGTTAAAAAC
AGGGAATAGACCTTATCGGATGACTCTAACAGACAATTGCTCCAG
GTCTTTCAGAGTTTTGAAAGATTATTTCAAGAAGGTAGATTTGGG
TTCTCTCAAGGTGGGCGGAATGGCTGCACAGTCAATGATTTCTCT
CTGGTTATATGGTGCCCACTCTGAATCCAACAGGAGCCGGAGATG
TATAACAGACTTGGCCCATTTCTATTCCAAGTCGTCCCCCATAGA
GAAGCTGTTGAATCTCACGCTAGGAAATAGAGGGCTGAGAATCCC
CCCAGAGGGAGTGTTAAGTTGCCTTGAGAGGGTTGATTATGATAA
TGCATTTGGAAGGTATCTTGCCAACACGTATTCCTCTTACTTGTT
CTTCCATGTAATCACCTTATACATGAACGCCCTAGACTGGGATGA
AGAAAAGACCATCCTAGCATTATGGAAAGATTTAACCTCAGTGGA
CATCGGGAAGGACTTGGTAAAGTTCAAAGACCAAATATGGGGACT
GCTGATCGTGACAAAGGACTTTGTTTACTCCCAAAGTTCCAATTG
TCTTTTTGACAGAAACTACACACTTATGCTAAAAGATCTTTTCTT
GTCTCGCTTCAACTCCTTAATGGTCTTGCTCTCTCCCCCAGAGCC
CCGATACTCAGATGACTTGATATCTCAACTATGCCAGCTGTACAT
TGCTGGGGATCAAGTCTTGTCTATGTGTGGAAACTCCGGCTATGA
AGTCATCAAAATATTGGAGCCATATGTCGTGAATAGTTTAGTCCA
GAGAGCAGAAAAGTTTAGGCCTCTCATTCATTCCTTGGGAGACTT
TCCTGTATTTATAAAAGACAAGGTAAGTCAACTTGAAGAGACGTT
CGGTCCCTGTGCAAGAAGGTTCTTTAGGGCTCTGGATCAATTCGA
CAACATACATGACTTGGTTTTTGTGTTTGGCTGTTACAGGCATTG
GGGGCACCCATATATAGATTATCGAAAGGGTCTGTCAAAACTATA
TGATCAGGTTCACCTTAAAAAAATGATAGATAAGTCCTACCAGGA
GTGCTTAGCAAGCGACCTAGCCAGGAGGATCCTTAGATGGGGTTT
TGATAAGTACTCCAAGTGGTATCTGGATTCAAGATTCCTAGCCCG
AGACCACCCCTTGACTCCTTATATCAAAACCCAAACATGGCCACC
CAAACATATTGTAGACTTGGTGGGGGATACATGGCACAAGCTCCC
GATCACGCAGATCTTTGAGATTCCTGAATCAATGGATCCGTCAGA
AATATTGGATGACAAATCACATTCTTTCACCAGAACGAGACTAGC
TTCTTGGCTGTCAGAAAACCGAGGGGGGCCTGTTCCTAGCGAAAA
AGTTATTATCACGGCCCTGTCTAAGCCGCCTGTCAATCCCCGAGA
GTTTCTGAGGTCTATAGACCTCGGAGGATTGCCAGATGAAGACTT
GATAATTGGCCTCAAGCCAAAGGAACGGGAATTGAAGATTGAAGG
TCGATTCTTTGCTCTAATGTCATGGAATCTAAGATTGTATTTTGT
CATCACTGAAAAACTCTTGGCCAACTACATCTTGCCACTTTTTGA
CGCGCTGACTATGACAGACAACCTGAACAAGGTGTTTAAAAAGCT
GATCGACAGGGTCACCGGGCAAGGGCTTTTGGACTATTCAAGGGT
CACATATGCATTTCACCTGGACTATGAAAAGTGGAACAACCATCA
AAGATTAGAGTCAACAGAGGATGTATTTTCTGTCCTAGATCAAGT
GTTTGGATTGAAGAGAGTGTTTTCTAGAACACACGAGTTTTTTCA
AAAGGCCTGGATCTATTATTCAGACAGATCAGACCTCATCGGGTT
ACGGGAGGATCAAATATACTGCTTAGATGCGTCCAACGGCCCAAC
CTGTTGGAATGGCCAGGATGGCGGGCTAGAAGGCTTACGGCAGAA
GGGCTGGAGTCTAGTCAGCTTATTGATGATAGATAGAGAATCTCA
AATCAGGAACACAAGAACCAAAATACTAGCTCAAGGAGACAACCA
GGTTTTATGTCCGACATACATGTTGTCGCCAGGGCTATCTCAAGA
GGGGCTCCTCTATGAATTGGAGAGAATATCAAGGAATGCACTTTC
GATATACAGAGCCGTCGAGGAAGGGGCATCTAAGCTAGGGCTGAT
CATCAAGAAAGAAGAGACCATGTGTAGTTATGACTTCCTCATCTA
TGGAAAAACCCCTTTGTTTAGAGGTAACATATTGGTGCCTGAGTC
CAAAAGATGGGCCAGAGTCTCTTGCGTCTCTAATGACCAAATAGT
CAACCTCGCCAATATAATGTCGACAGTGTCCACCAATGCGCTAAC
AGTGGCACAACACTCTCAATCTTTGATCAAACCGATGAGGGATTT
TCTGCTCATGTCAGTACAGGCAGTCTTTCACTACCTGCTATTTAG
CCCAATCTTAAAGGGAAGAGTTTACAAGATTCTGAGCGCTGAAGG
GGAGAGCTTTCTCCTAGCCATGTCAAGGATAATCTATCTAGATCC
TTCTTTGGGAGGGATATCTGGAATGTCCCTCGGAAGATTCCATAT
ACGACAGTTCTCAGACCCTGTCTCTGAAGGGTTATCCTTCTGGAG
AGAGATCTGGTTAAGCTCCCAAGAGTCCTGGATTCACGCGTTGTG
TCAAGAGGCTGGAAACCCAGATCTTGGAGAGAGAACACTCGAGAG
CTTCACTCGCCTTCTAGAAGATCCGACCACCTTAAATATCAGAGG
AGGGGCCAGTCCTACCATTCTACTCAAGGATGCAATCAGAAAGGC
TTTATATGACGAGGTGGACAAGGTGGAAAATTCAGAGTTTCGAGA
GGCAATCCTGTTGTCCAAGACCCATAGAGATAATTTTATACTCTT
CTTAATATCTGTTGAGCCTCTGTTTCCTCGATTTCTCAGTGAGCT
ATTCAGTTCGTCTTTTTTGGGAATCCCCGAGTCAATCATTGGATT
GATACAAAACTCCCGAACGATAAGAAGGCAGTTTAGAAAGAGTCT
CTCAAAAACTTTAGAAGAATCCTTCTACAACTCAGAGATCCACGG
GATTAGTCGGATGACCCAGACACCTCAGAGGGTTGGGGGGGTGTG
GCCTTGCTCTTCAGAGAGGGCAGATCTACTTAGGGAGATCTCTTG
GGGAAGAAAAGTGGTAGGCACGACAGTTCCTCACCCTTCTGAGAT
GTTGGGATTACTTCCCAAGTCCTCTATTTCTTGCACTTGTGGAGC
AACAGGAGGAGGCAATCCTAGAGTTTCTGTATCAGTACTCCCGTC
CTTTGATCAGTCATTTTTTTCACGAGGCCCCCTAAAGGGATACTT
GGGCTCGTCCACCTCTATGTCGACCCAGCTATTCCATGCATGGGA
AAAAGTCACTAATGTTCATGTGGTGAAGAGAGCTCTATCGTTAAA
AGAATCTATAAACTGGTTCATTACTAGAGATTCCAACTTGGCTCA
AGCTCTAATTAGGAACATTATGTCTCTGACAGGCCCTGATTTCCC
TCTAGAGGAGGCCCCTGTCTTCAAAAGGACGGGGTCAGCCTTGCA
TAGGTTCAAGTCTGCCAGATACAGCGAAGGAGGGTATTCTTCTGT
CTGCCCGAACCTCCTCTCTCATATTTCTGTTAGTACAGACACCAT
GTCTGATTTGACCCAAGACGGGAAGAACTACGATTTCATGTTCCA
GCCATTGATGCTTTATGCACAGACATGGACATCAGAGCTGGTACA
GAGAGACACAAGGCTAAGAGACTCTACGTTTCATTGGCACCTCCG
ATGCAACAGGTGTGTGAGACCCATTGACGACGTGACCCTGGAGAC
CTCTCAGATCTTCGAGTTTCCGGATGTGTCGAAAAGAATATCCAG
AATGGTTTCTGGGGCTGTGCCTCACTTCCAGAGGCTTCCCGATAT
CCGTCTGAGACCAGGAGATTTTGAATCTCTAAGCGGTAGAGAAAA
GTCTCACCATATCGGATCAGCTCAGGGGCTCTTATACTCAATCTT
AGTGGCAATTCACGACTCAGGATACAATGATGGAACCATCTTCCC
TGTCAACATATACGGCAAGGTTTCCCCTAGAGACTATTTGAGAGG
GCTCGCAAGGGGAGTATTGATAGGATCCTCGATTTGCTTCTTGAC
AAGAATGACAAATATCAATATTAATAGACCTCTTGAATTGGTCTC
AGGGGTAATCTCATATATTCTCCTGAGGCTAGATAACCATCCCTC
CTTGTACATAATGCTCAGAGAACCGTCTCTTAGAGGAGAGATATT
TTCTATCCCTCAGAAAATCCCCGCCGCTTATCCAACCACTATGAA
AGAAGGCAACAGATCAATCTTGTGTTATCTCCAACATGTGCTACG
CTATGAGCGAGAGATAATCACGGCGTCTCCAGAGAATGACTGGCT
ATGGATCTTTTCAGACTTTAGAAGTGCCAAAATGACGTACCTATC
CCTCATTACTTACCAGTCTCATCTTCTACTCCAGAGGGTTGAGAG
AAACCTATCTAAGAGTATGAGAGATAACCTGCGACAATTGAGTTC
TTTGATGAGGCAGGTGCTGGGCGGGCACGGAGAAGATACCTTAGA
GTCAGACGACAACATTCAACGACTGCTAAAAGACTCTTTACGAAG
GACAAGATGGGTGGATCAAGAGGTGCGCCATGCAGCTAGAACCAT
GACTGGAGATTACAGCCCCAACAAGAAGGTGTCCCGTAAGGTAGG
ATGTTCAGAATGGGTCTGCTCTGCTCAACAGGTTGCAGTCTCTAC
CTCAGCAAACCCGGCCCCTGTCTCGGAGCTTGACATAAGGGCCCT
CTCTAAGAGGTTCCAGAACCCTTTGATCTCGGGCTTGAGAGTGGT
TCAGTGGGCAACCGGTGCTCATTATAAGCTTAAGCCTATTCTAGA
TGATCTCAATGTTTTCCCATCTCTCTGCCTTGTAGTTGGGGACGG
GTCAGGGGGGATATCAAGGGCAGTCCTCAACATGTTTCCAGATGC
CAAGCTTGTGTTCAACAGTCTTTTAGAGGTGAATGACCTGATGGC
TTCCGGAACACATCCACTGCCTCCTTCAGCAATCATGAGGGGAGG
AAATGATATCGTCTCCAGAGTGATAGATCTTGACTCAATCTGGGA
AAAACCGTCCGACTTGAGAAACTTGGCAACCTGGAAATACTTCCA
GTCAGTCCAAAAGCAGGTCAACATGTCCTATGACCTCATTATTTG
CGATGCAGAAGTTACTGACATTGCATCTATCAACCGGATCACCCT
GTTAATGTCCGATTTTGCATTGTCTATAGATGGACCACTCTATTT
GGTCTTCAAAACTTATGGGACTATGCTAGTAAATCCAAACTACAA
GGCTATTCAACACCTGTCAAGAGCGTTCCCCTCGGTCACAGGGTT
TATCACCCAAGTAACTTCGTCTTTTTCATCTGAGCTCTACCTCCG
ATTCTCCAAACGAGGGAAGTTTTTCAGAGATGCTGAGTACTTGAC
CTCTTCCACCCTTCGAGAAATGAGCCTTGTGTTATTCAATTGTAG
CAGCCCCAAGAGTGAGATGCAGAGAGCTCGTTCCTTGAACTATCA
GGATCTTGTGAGAGGATTTCCTGAAGAAATCATATCAAATCCTTA
CAATGAGATGATCATAACTCTGATTGACAGTGATGTAGAATCTTT
TCTAGTCCACAAGATGGTTGATGATCTTGAGTTACAGAGGGGAAC
TCTGTCTAAAGTGGCTATCATTATAGCCATCATGATAGTTTTCTC
CAACAGAGTCTTCAACGTTTCCAAACCCCTAACTGACCCCTCGTT
CTATCCACCGTCTGATCCCAAAATCCTGAGGCACTTCAACATATG
TTGCAGTACTATGATGTATCTATCTACTGCTTTAGGTGACGTCCC
TAGCTTCGCAAGACTTCACGACCTGTATAACAGACCTATAACTTA
TTACTTCAGAAAGCAAGTCATTCGAGGGAACGTTTATCTATCTTG
GAGTTGGTCCAACGACACCTCAGTGTTCAAAAGGGTAGCCTGTAA
TTCTAGCCTGAGTCTGTCATCTCACTGGATCAGGTTGATTTACAA
GATAGTGAAGACTACCAGACTCGTTGGCAGCATCAAGGATCTATC
CAGAGAAGTGGAAAGACACCTTCATAGGTACAACAGGTGGATCAC
CCTAGAGGATATCAGATCTAGATCATCCCTACTAGACTACAGTTG
CCTGTGAACCGGATACTCCTGGAAGCCTGCCCATGCTAAGACTCT
TGTGTGATGTATCTTGAAAAAAACAAGATCCTAAATCTGAACCTT
TGGTTGTTTGATTGTTTTTCTCATTTTTGTTGTTTATTTGTTAAG
CGT

The nucleoprotein (N) of SAD B19 (mRNA encoded by nucleotides 59-1482 of SEQ ID NO:73 and CDS at nucleotides 71-1423 encodes the following protein sequence (GenBank Accession No. AAA47199.1; SEQ ID NO:74):

MDADKIVFKVNNQVVSLKPEIIVDQYEYKYPAIKDLKKPCITLGK
APDLNKAYKSVLSGMSAAKLNPDDVCSYLAAAMQFFEGTCPEDWT
SYGIVIARKGDKITPGSLVEIKRTDVEGNWALTGGMELTRDPTVP
EHASLVGLLLSLYRLSKISGQNTGNYKTNIADRIEQIFETAPFVK
IVEHHTLMTTHKMCANWSTIPNFRFLAGTYDMFFSRIEHLYSAIR
VGTVVTAYEDCSGLVSFTGFIKQINLTAREAILYFFHKNFEEEIR
RMFEPGQETAVPHSYFIHFRSLGLSGKSPYSSNAVGHVENLIHFV
GCYMGQVRSLNATVIAACAPHEMSVLGGYLGEEFFGKGTFERRFF
RDEKELQEYEAAELTKTDVALADDGTVNSDDEDYFSGETRSPEAV
YTRIMMNGGRLKRSHIRRYVSVSSNHQARPNSFAEFLNKTYSSDS

The phosphoprotein (P) of SAD B19 (mRNA encoded by nucleotides 1485-2475 of SEQ ID NO:73 and CDS at nucleotides 1514-2407 encodes the following protein sequence (GenBank Accession No. AAA47200.1; SEQ ID NO:75):

MSKIFVNPSAIRAGLADLEMAEETVDLINRNIEDNQAHLQGEPIE
VDNLPEDMGRLHLDDGKSPNHGEIAKVGEGKYREDFQMDEGEDPS
FLFQSYLENVGVQIVRQMRSGERFLKIWSQTVEEIISYVAVNFPN
PPGKSSEDKSTQTTGRELKKETTPTPSQRESQSSKARMAAQIASG
PPALEWSATNEEDDLSVEAEIAHQIAESFSKKYKFPSRSSGILLY
NFEQLKMNLDDIVKEAKNVPGVTRLAHDGSKLPLRCVLGWVALAN
SKKFQLLVESDKLSKIMQDDLNRYTSC

The matrix protein (M) of SAD B19 (mRNA encoded by nucleotides 2481-3284 of SEQ ID NO:73 and CDS at nucleotides 2496-3104 encodes the following protein sequence (GenBank 35 Accession No. AAA47201.1; SEQ ID NO:76):

MNLLRKIVKNRRDEDTQKSSPASAPLDDDDLWLPPPEYVPLKELT
GKKNMRNFCINGRVKVCSPNGYSFRILRHILKSFDEIYSGNHRMI
GLVKVVIGLALSGSPVPEGLNWVYKLRRTFIFQWADSRGPLEGEE
LEYSQEITWDDDTEFVGLQIRVIAKQCHIQGRVWCINMNPRACQL
WSDMSLQTQRSEEDKDSSLLLE

The glycoprotein (G) of SAD B19 (mRNA encoded by nucleotides 3290-5359 of SEQ ID NO:73 and CDS at nucleotides 3317-4891 encodes the following protein sequence (GenBank Accession No. AAA47202.1; SEQ ID NO:77):

MVPQALLFVPLLVFPLCFGKFPIYTIPDKLGPWSPIDIHHLSCPN
LVVEDEGCTNLSGFSYMELKVGYILAIKVNGFTCTGVVTEAETYT
NFVGYVTTTFKRKHFRPTPDACRAAYNWKMAGDPRYEESLHNPYP
DYRWLRTVKTTKESLVIISPSVADLDPYDRSLHSRVFPSGKCSGV
AVSSTYCSTNHDYTIWMPENPRLGMSCDIFTNSRGKRASKGSETC
GFVDERGLYKSLKGACKLKLCGVLGLRLMDGTWVSMQTSNETKWC
PPDKLVNLHDFRSDEIEHLVVEELVRKREECLDALESIMTTKSVS
FRRLSHLRKLVPGFGKAYTIFNKTLMEADAHYKSVRTWNEILPSK
GCLRVGGRCHPHVNGVFFNGIILGPDGNVLIPEMQSSLLQQHMEL
LESSVIPLVHPLADPSTVFKDGDEAEDFVEVHLPDVHNQVSGVDL
GLPNWGKYVLLSAGALTALMLIIFLMTCCRRVNRSEPTQHNLRGT
GREVSVTPQSGKIISSWESHKSGGETRL

The polymerase (L) of SAD B19 (mRNA encoded by nucleotides 5384-11858 of SEQ ID NO:73 and CDS at nucleotides 5414-11797 encodes the following protein sequence (GenBank Accession No. AAA47203.1; SEQ ID NO:78):

MLDPGEVYDDPIDPIELEAEPRGTPIVPNILRNSDYNLNSPLIED
PARLMLEWLKTGNRPYRMTLTDNCSRSFRVLKDYFKKVDLGSLKV
GGMAAQSMISLWLYGAHSESNRSRRCITDLAHFYSKSSPIEKLLN
LTLGNRGLRIPPEGVLSCLERVDYDNAFGRYLANTYSSYLFFHVI
TLYMNALDWDEEKTILALWKDLTSVDIGKDLVKFKDQIWGLLIVT
KDFVYSQSSNCLFDRNYTLMLKDLFLSRENSLMVLLSPPEPRYSD
DLISQLCQLYIAGDQVLSMCGNSGYEVIKILEPYVVNSLVQRAEK
FRPLIHSLGDFPVFIKDKVSQLEETFGPCARRFFRALDQFDNIHD
LVFVFGCYRHWGHPYIDYRKGLSKLYDQVHLKKMIDKSYQECLAS
DLARRILRWGFDKYSKWYLDSRFLARDHPLTPYIKTQTWPPKHIV
DLVGDTWHKLPITQIFEIPESMDPSEILDDKSHSFTRTRLASWLS
ENRGGPVPSEKVIITALSKPPVNPREFLRSIDLGGLPDEDLIIGL
KPKERELKIEGRFFALMSWNLRLYFVITEKLLANYILPLFDALTM
TDNLNKVFKKLIDRVTGQGLLDYSRVTYAFHLDYEKWNNHQRLES
TEDVFSVLDQVFGLKRVFSRTHEFFQKAWIYYSDRSDLIGLREDQ
IYCLDASNGPTCWNGQDGGLEGLRQKGWSLVSLLMIDRESQIRNT
RTKILAQGDNQVLCPTYMLSPGLSQEGLLYELERISRNALSIYRA
VEEGASKLGLIIKKEETMCSYDFLIYGKTPLFRGNILVPESKRWA
RVSCVSNDQIVNLANIMSTVSTNALTVAQHSQSLIKPMRDFLLMS
VQAVFHYLLESPILKGRVYKILSAEGESFLLAMSRIIYLDPSLGG
ISGMSLGRFHIRQFDPVSEGLSFWREIWLSSQESWIHALCQEAGN
PDLGERTLESFTRLLEDPTTLNIRGGASPTILLKDAIRKALYDEV
DKVENSEFREAILLSKTHRDNFILFLISVEPLFPRFLSELFSSSF
LGIPESIIGLIQNSRTIRRQFRKSLSKTLEESFYNSEIHGISRMT
QTPQRVGGVWPCSSERADLLREISWGRKVVGTTVPHPSEMLGLLP
KSSISCTCGATGGGNPRVSVSVLPSEDQSFFSRGPLKGYLGSSTS
MSTQLFHAWEKVINVHVVKRALSLKESINWFITRDSNLAQALIRN
IMSLTGPDFPLEEAPVFKRTGSALHRFKSARYSEGGYSSVCPNLL
SHISVSTDTMSDLTQDGKNYDEMFQPLMLYAQTWTSELVQRDTRL
RDSTFHWHLRCNRCVRPIDDVTLETSQIFEFPDVSKRISRMVSGA
VPHFQRLPDIRLRPGDFESLSGREKSHHIGSAQGLLYSILVAIHD
SGYNDGTIFPVNIYGKVSPRDYLRGLARGVLIGSSICFLTRMINI
NINRPLELVSGVISYILLRLDNHPSLYIMLREPSLRGEIFSIPQK
IPAAYPTTMKEGNRSILCYLQHVLRYEREIITASPENDWLWIFSD
FRSAKMTYLSLITYQSHLLLQRVERNLSKSMRDNLRQLSSLMRQV
LGGHGEDTLESDDNIQRLLKDSLRRTRWVDQEVRHAARTMTGDYS
PNKKVSRKVGCSEWVCSAQQVAVSTSANPAPVSELDIRALSKRFQ
NPLISGLRVVQWATGAHYKLKPILDDLNVFPSLCLVVGDGSGGIS
RAVLNMFPDAKLVENSLLEVNDLMASGTHPLPPSAIMRGGNDIVS
RVIDLDSIWEKPSDLRNLATWKYFQSVQKQVNMSYDLIICDAEVT
DIASINRITLLMSDFALSIDGPLYLVFKTYGTMLVNPNYKAIQHL
SRAFPSVTGFITQVTSSFSSELYLRFSKRGKFFRDAEYLTSSTLR
EMSLVLFNCSPKSEMQRARSLNYQDLVRGEPEEIISNPYNEMIIT
LIDSDVESFLVHKMVDDLELQRGTLSKVAIIIAIMIVFSNRVENV
SKPLTDPSFYPPSDPKILRHFNICCSTMMYLSTALGDVPSFARLH
DLYNRPITYYFRKQVIRGNVYLSWSWSNDTSVFKRVACNSSLSLS
SHWIRLIYKIVKTTRLVGSIKDLSREVERHLHRYNRWITLEDIRS
RSSLLDYSCL

The recombinant rabies virus can include one or more deletions or inactivating mutation in one or more genes, e.g., genetic deletion(s) such as those described herein. For example, when the G protein is functionally deleted, the release of viral particles is significantly reduced. Since G is required for transneuronal transfer of Rabies virus, RabΔG cannot spread without trans-complementation, so it cannot spread from cells that don't express the G protein (Etessami et al., J Gen Virol. 2000 Sep; 81(Pt 9):2147-2153; Wall et al., PNAS December 14, 2010 107 (50) 21848-21853; Suzuki et al., Front Neural Circuits. 2020 Jan 10;13:77; Callaway and Luo, J Neurosci. 2015 Jun 17; 35(24): 8979-8985). In some embodiments, the inactivating mutation is in the G protein gene. Thus, in some embodiments, the recombinant rabies virus does not encode a functional G protein.

Other non-limiting examples of suitable viral tracers include:

| Tracer | Description | Reference |
|---|---|---|
| 2^{nd}-gen RV (RVΔGL-4 or -1) | A double deletion mutant | Chatterjee et al., Nature |
| | rabies virus to prevent | Neuroscience 2018; |
| | cytotoxicity | doi: 10.1038/s41593-018- |
| | | 0091-7. |
| AAV1 and AAV9 | Specific AAV serotypes that | Zingg et al., Neuron 2017; |
| anterograde transsynaptic | spread anterogradely and | doi: |
| tracing | label interacting cells | 10.1016/j.neuron.2016.11.045 |
| HSV-1 Strain H129DTK-TT | Herpes simplex virus that | Lo and Anderson, Neuron |
| | spreads anterogradely and | 2011; DOI: |
| | can be Cre-dependent | 10.1016/j.neuron.2011.12.002 |

### Pseudotypes

In some embodiments, the recombinant rabies virus is a pseudovirus, e.g., is pseudotyped, e.g., comprises a foreign virus envelope protein, e.g., a non-rabies virus G protein. See, e.g., Sanders, “No False Start for Novel Pseudotyped Vectors,” Current Opinion in Biotechnology 13:437-42 (2002) and Joglekar and Sandoval, “Pseudotyped Lentiviral Vectors: One Vector, Many Guises,” Human Gene Therapy Methods 28(6):291-301 (2017).

Thus, in some embodiments, the recombinant rabies virus comprises a gene encoding a foreign virus envelope protein. In some embodiments, the foreign virus envelope protein is selected from the group consisting of a retrovirus envelope protein, a paramyxovirus envelope protein, an alphavirus envelope protein, an orthomyxovirus envelope protein, a vesiculovirus envelope protein, and combinations thereof.

In some embodiments, the recombinant rabies virus comprises a gene selected from HIV envelope glycoprotein p160, HIV envelope glycoprotein gp120, HIV envelope glycoprotein gp41, retrovirus envelope protein gp70, retrovirus envelope protein SU, retrovirus envelope protein TM, retrovirus envelope protein A, retrovirus envelope protein CVS-G paramyxovirus envelope protein H, paramyxovirus envelope protein F, rhabdovirus envelope protein G, rhabdovirus envelope protein GP1, rhabdovirus envelope protein GP2, rhabdovirus envelope protein GP64, alphavirus envelope protein El, alphavirus envelope protein E2, orthomyxovirus envelope protein HA, vesiculovirus envelope protein VSV-G, vesiculovirus envelope protein CNV-G, vesiculovirus envelope protein PRV-G, and combinations thereof.

In some embodiments, the retrovirus envelope protein A is avian sarcoma leucosis virus glycoprotein EnvA (Wall et al., PNAS December 14,2010 107 (50) 21848-21853; Suzuki et al., Front Neural Circuits. 2020 Jan 10;13:77; Callaway and Luo, J Neurosci. 2015 Jun 17; 35(24): 8979-8985). In some embodiments, the retrovirus envelope protein CSV-G is RV CVSG. See, e.g., Hagendorf and Conzelmann, “Pseudotyping of G-Gene-Deficient Rabies Virus,” Cold Spring Harbor Prtoco. Doi:10.1101/pdb.prot069417 (2015).

### Variants

In some embodiments, the recombinant rabies virus described herein and/or the components of the recombinant rabies virus described herein, e.g., genes encoded by the recombinant rabies viruses described herein, are at least 80%, e.g., at least 85%, 90%, 95%, 97%, or 99% identical to the nucleic or amino acid sequence of an exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the mutations described herein. In some embodiments, the variant gene(s) encoded by the recombinant rabies virus retains the same gene function as the parent gene.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M.O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Also provided herein are nucleic acids encoding the recombinant rabies viruses and/or components thereof described herein, e.g., isolated nucleic acids encoding the recombinant rabies viruses and/or components thereof, vectors comprising nucleic acids, optionally operably linked to one or more regulatory domains for expressing the proteins encoded by the nucleic acids, and host cells, e.g., mammalian host cells, comprising the nucleic acids and/or vectors.

### Reporter Genes

In some embodiments, the recombinant rabies virus comprises a reporter gene. In some embodiments, the reporter gene is a gene that encodes a fluorescent protein. In some embodiments, the reporter gene encodes a green fluorescent protein, a red fluorescent protein, a yellow fluorescent protein, a blue fluorescent protein, a cyan fluorescent protein, an orange fluorescent protein, or combinations thereof.

In some embodiments, reporter gene encodes a green fluorescent protein selected from GFP, EGFP, Emerland, Superfold GFP, Azami Green, mWasabi, TagGFP, TurboGFP, AcGFP, ZsGreen, T-Sapphire, click beetle green, and combinations thereof.

In some embodiments, the reporter gene encodes a blue fluorescent protein selected from EBFP, EBFP2, Azurite, mTagBFP, and combinations thereof.

In some embodiments, the reporter gene encodes a cyan fluorescent protein selected from ECFP, mECFP, Cerulean, mTurqoise, CyPet, AmCyan1, Midori-Ishi Cyan, TagCFP, mTFP11 (Teal), and combinations thereof.

In some embodiments, the reporter gene encodes a yellow fluorescent protein selected from EYFP, Topaz, Venus, mCitrine, YPet, TagYFP, PhiYFP, ZsYellow1, mBanana, and combinations thereof.

In some embodiments, the reporter gene encodes an orange fluorescent protein selected from Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine, and combinations thereof.

In some embodiments, the reporter gene encodes a red fluorescent protein selected from mRuby, mApple, mStrawberry, AsRed2, mRFP1, JRed, mCherry, HcRed1, mRaspberry, dKeima-Tandem, HcRed-Tandem, mPlum, AQ143, or combinations thereof.

### Virus Libraries

Generally speaking, the recombinant rabies viruses described herein comprise barcodes (e.g., exogenous nucleotide sequences, orthogonal to the rabies virus genome and/or the other components of the recombinant rabies viruses described here) that can be used to differentiate between different recombinant rabies viruses).

In some cases, the barcode is from 10-500 nts long, e.g., 10-450, 10-400, 10-350, 10-300, 10-250, 10-200, 10-150, 10-100, 10-50, 10-40, 10-30, 10-20, 20-450, 20-400, 20-350, 20-300, 20-250, 20-200, 20-150, 20-100, 20-50, 20-40, 20-30, 30-500, 30-450, 30-400, 30-350, 30-300, 30-250, 30-200, 30-150, 30-100, 30-50, 30-40, 40-500, 40-450, 40-400, 40-350, 40-300, 40-250, 40-200, 40-150, 40-100, 40-50, 50-500, 50-450, 50-400, 50-350, 50-300, 50-250, 50-200, 50-150, 50-100, 100-500, 100-450, 100-400, 100-350, 100-300, 100-250, 100-200, 100-150, 150-500, 150-450, 150-400, 150-350, 150-300, 150-250, 150-200, 200-500, 200-450, 200-400, 200-350, 200-300, 200-250, 250-500, 250-450, 250-400, 250-350, 250-300, 300-500, 300-450, 300-400, 300-350, 350-500, 350-450, 350-400, 400-500, 400-450, or 450-500 nts long.

Each virus can includes barcodes that include one sequence, or two or more copies of a repeated sequence, e.g., between 2 and 10 copies of the repeated sequence, optionally 1, 2, 3, 4, 5, 6, 7, 8, 9, or 10 copies of the repeated sequence. The barcodes can include a spacer between repeated barcode sequence(s). In some embodiments, the spacer comprises 1, 2, 3, 4, 5, 6, 7, 8, 9, or 10 nucleotides. In some embodiments, the spacer comprises one or more AT (Adenine-Thymine) dinucleotide sequences.

In some embodiments, each repeated sequence in the barcode is separated by a spacer. When multiple spacers are present in a single virus, the spacers can be the same, or the spacers can be different. When present in a plurality of viruses with different barcodes (e.g., a library), the spacers can be the same in every virus, or the spacers can be different, e.g., unique to every unique virus.

In some embodiments, the barcode comprises the nucleotide sequence VHDBVHDB (SEQ ID NO:2), according to IUPAC ambiguity codes, where V is any nucleotide except T, H is any nucleotide except G, D is any nucleotide except C, and B is any nucleotide except A.

In some embodiments, the barcode comprises repeats of the nucleotide sequence VHDBVHDB (SEQ ID NO:2).

In some embodiments, the barcode comprises spacers between the repeats of the nucleotide sequence VHDBVHDB (SEQ ID NO:2). In some embodiments, the spacer between the repeats of the nucleotide sequence VHDBVHDB (SEQ ID NO:2) is AT.

In some embodiments, the barcode sequence comprises the nucleotide sequence VHDBVHDBATVHDBVHDBATVHDBVHDB (SEQ ID NO:1). In some embodiments, the barcode sequence is VHDBVHDBATVHDBVHDBATVHDBVHDB (SEQ ID NO:1).

In some embodiments, the plurality of viruses comprises 1.4e7 to 1.4e18 different barcodes

In some embodiments, the recombinant rabies viruses of the plurality further comprise a first constant region 5′ of the barcode and a second constant region 3′ of the barcode. In some cases, the first constant region and second constant region is each, independently, from 10-500 nts long, e.g., 10-450, 10-400, 10-350, 10-300, 10-250, 10-200, 10-150, 10-100, 10-50, 10-40, 10-30, 10-20, 20-450, 20-400, 20-350, 20-300, 20-250, 20-200, 20-150, 20-100, 20-50, 20-40, 20-30, 30-500, 30-450, 30-400, 30-350, 30-300, 30-250, 30-200, 30-150, 30-100, 30-50, 30-40, 40-500, 40-450, 40-400, 40-350, 40-300, 40-250, 40-200, 40-150, 40-100, 40-50, 50-500, 50-450, 50-400, 50-350, 50-300, 50-250, 50-200, 50-150, 50-100, 100-500, 100-450, 100-400, 100-350, 100-300, 100-250, 100-200, 100-150, 150-500, 150-450, 150-400, 150-350, 150-300, 150-250, 150-200, 200-500, 200-450, 200-400, 200-350, 200-300, 200-250, 250-500, 250-450, 250-400, 250-350, 250-300, 300-500, 300-450, 300-400, 300-350, 350-500, 350-450, 350-400, 400-500, 400-450, or 450-500 nts long.

In some embodiments, the first constant region is conserved amongst the members of the plurality. In some embodiments, the second constant region is conserved amongst the members of the plurality. In some embodiments, both the first and second constant regions are conserved amongst members of the plurality.

In some cases, the first and/or second constant region(s) comprise per priming sites.

In some embodiments, the barcode and/or constant regions are encoded on the mRNA that also encodes a reporter gene (i.e., part of the same coding region); for example, the barcode sequences can be inserted prior to the transcriptional stop of the reporter gene transcript, so that the transcribed mRNA barcode can be analyzed by scRNA-seq. In some embodiments, mRNA that encodes the barcode and/or constant region further comprises a 3′ polyadenylated tail. The barcode sequence can be just 5′ of the polyadenylation sequence.

Also described herein are recombinant virus libraries comprising a plurality of viruses, e.g., a plurality of recombinant rabies viruses, having a plurality of different barcodes.

### Methods for Tracing Cell Networks

The recombinant rabies viruses described herein can be used in methods for tracing cell networks. The methods can be applied to any type of cell network that can be targeted by the recombinant rabies viruses described herein, e.g. organ system(s) and tumor cell-host networks

In some embodiments, the cell network comprises CNS cells, e.g., glial cells (e.g., astrocytes, microglia, oligodendrocytes, and/or ependymal cells) and/or neurons.

In some cases, the cell network is in a living mammal (i.e., a non-human mammal).

In some cases, the cell network is outside of a living mammal. In some cases, the cell network is a cell and/or tissue sample from a living mammal. In some cases, the cell network is a mammalian cell culture. In some cases, the cell network is a xenoplanted cell network (e.g., a tumor xenograph). In some cases, the cell network is an organoid.

In some cases, cell network comprises tumor cells. In some cases, the tumor is a CNS tumor. See, e.g., Louis et al., “The 2016 World Health Organization Classification of Tumors of the Central Nervous System: a summary,” Acta Neuropathologica 131:803-20 (2016), which is hereby incorporated by reference in its entirety. In some cases, the CNS tumor is a spinal cord tumor. In some cases, the CNS tumor is a brain tumor. In some cases, the brain tumor is a glioblastoma.

In some cases, the mammal is a human. In some cases, the mammal is a non-human mammal, e.g., a non-human primate, rat, or mouse.

The methods can include expressing one or more proteins in a cell- or tissue-specific manner that restricts the cell type that can be directly infected with the rabies virus. For example, the cells can be engineered to express TVA, an avian receptor protein that allows infection with rabies virus pseudotyped with the avian sarcoma leucosis virus glycoprotein EnvA (Wall et al., PNAS December 14, 2010 107 (50) 21848-21853; Suzuki et al., Front Neural Circuits. 2020 Jan 10;13:77; Callaway and Luo, J Neurosci. 2015 Jun 17; 35(24): 8979-8985). In addition, the cells are engineered to express G proteins (again in a cell- or tissue-specific manner), such that the expressed G proteins complement the missing G protein in the rabies virus, and allow transmission only from the infected cells that express the G proteins. In this way, great specificity can be obtained. The same or different cell- or tissue-specific promoters can be used for the TVA and G proteins, so long as they direct expression to the same cells. In some embodiments, the cells are in an animal that expresses TVA and/or G protein in only a defined set of cells, or expresses TVA or G protein ubiquitously (e.g., in substantially all of the cells of the animal). The methods can be used in in vitro models of cellular networks as well. A number of cell- and tissue-specific promoters are known in the art, including, e.g., Cre drivers described in the JAX Cre Repository (jax.org/research-and-faculty/resources/cre-repository).

In some cases, the method includes transduction, e.g., viral transduction, e.g., lentiviral transduction, of a cell network with a vector, e.g., a viral vector such as an AAV or lentiviral vector, that comprises a nucleic acid sequence, preferably comprising a cell- or tissue-specific promoter, a nucleic acid sequence encoding TVA, and a nucleic acid sequence encoding G. In some cases, the vector further comprises one or more nucleic acid sequences encoding a self-cleaving peptide, e.g., a 2A self-cleaving peptide. In some cases, the nucleic acid sequences encoding the self-cleaving peptide(s) are interleaved between the nucleic acid sequence(s) encoding the TVA, G, and/or EGFP.

In some cases, the cell-or tissue-specific promoter is a mammalian cell- or tissue-specific promoter.

Suitable cell- or tissue-specific promoters include, but are not limited to those shown in Table A, below.

TABLE A
Cell- and Tissue-Specific Promoters

| | | Tissue | | |
|---|---|---|---|---|
| Name | Description | Specificity | Cell Type Specificity | References |
| Nanog | Mouse nanog | Embryonic | Pluripotent stem cells | J Biol Chem. |
| | promoter | inner cell | such as embryonic stem | 280: 24731 (2005) |
| | | mass | cells | |
| Nes | Rat nestin intron | Nervous system | Neural stem/progenitor | Eur J Neurosci. |
| | 2 enhancer fused | at embryonic | cells | 9: 452 (1997); J |
| | to mouse Hsp68 | stage | | Neurosci Res. |
| | minimal promoter | | | 59:321 (2000) |
| Tuba1a | Rat α1A tubulin | Nervous systems | Developing neurons | J Neurosci. |
| | promoter | at embryonic and | but not astrocytes or | 14: 7319 (1994) |
| | | early postnatal | oligodendrocytes | |
| | | stages | | |
| Camk2a | Mouse α-calcium- | Forebrain | CA1 pyramidal cells | Cell. 81: 891 |
| (long) | calmodulin | | of the hippocampus | (1995); Cell. |
| | dependent kinase | | | 87: 1317 (1996) |
| | II promoter (8 kb) | | | |
| Camk2a | Mouse α-calcium- | Brain | Pyramidal neurons | Neuroscience. |
| (short) | calmodulin | | | 161: 441 (2009) |
| | dependent kinase | | | |
| | II promoter (1.3 kb) | | | |
| SYN1 | Human synapsin I | Brain | Mature neurons | Mol Ther. 5: 509 |
| | promoter | | | (2002) |
| Hb9 | Mouse Hb9 | Spinal cord | Motor neurons | Exp Neurol. 196: 224 |
| | enhancer fused to | | | (2005); Dev Biol. |
| | mouse Hsp68 | | | 283: 474 (2005) |
| | minimal promoter | | | |
| Th | Mouse tyrosine | Brain | Dopaminergic neurons | Biochem Biophys |
| | hydroxylase | | | Res Commun. 274: 590 |
| | promoter | | | (2000); Neurosci |
| | | | | Lett. 363: 33 (2004) |
| NSE | Rat neuron- | Nervous | Various neurons | Neuron. 5: 187 |
| | specific enolase | system | | (1990); J Mol |
| | promoter | | | Neurosci. 8: 63(1997) |
| GFAP | Human glial | Brain | Astrocytes | J Neurosci. |
| (long) | fibrillary acidic | | | 14: 1030 (1994) |
| | protein promoter | | | |
| | (2.1 kb) | | | |
| GFAP | Human glial | Brain | Astrocytes | Glia. 56: 481 |
| (short) | fibrillary acidic | | | (2008) |
| | protein promoter | | | |
| | (0.68 kb) | | | |
| Iba1 | Mouse ionized | Brain | Microglia | J Neurosci Res. |
| | calcium binding | | | 81: 357 (2005) |
| | adapter molecule | | | |
| | 1 promoter | | | |
| ProA1 | Mouse Gnat2 | Retina | Cone photoreceptor | Nat Neurosci. 22: 1345 |
| | promoter | | | (2019); Int J Mol |
| | | | | Sci. 21: 4197 (2020) |
| hRHO | Human rhodopsin | Retina | Rod photoreceptor | J Virol. 81: 11372 |
| | promoter | | | (2007); eLife. 5: |
| | | | | e12242 (2016) |
| hBEST1 | Human bestrophin 1 | Retina | RPE | Hum Mol Genet. |
| | promoter | | | 18: 128 (2009) |
| Prnp | Mouse prion | Brain, | Various CNS neurons | Eur J Neurosci. |
| | protein promoter | hippocampus, | | 14: 1777 (2001) |
| | | cerebellum, | | |
| | | retina | | |
| Cnp | Mouse 2′,3′-cyclic | Nervous | Oligodendrocytes and | J Neurosci Res. |
| | nucleotide 3′- | systems, testis | Schwann cells | 53: 393 (1998); J |
| | phosphodiesterase | and thymus | | Neurosci. 19: 759 |
| | promoter | | | (1999) |
| K14 | Human keratin 14 | Epidermis | Keratinocytes | Gene. 153: 297 |
| | promoter | | | (1995) |
| BK5 | Bovine keratin 5 | Epidermis | Keratinocytes | Differentiation. |
| | promoter | | | 58: 53 |
| | | | | (1994); Oncogene. |
| | | | | 19: 4243 (2000) |
| mTyr | Mouse tyrosinase | Epidermis | Melanocytes and | Development. 120: 2103 |
| | promoter and | | melanoma cells | (1994); Gene Ther. |
| | enhancer | | | 1: 307 (1994); Int J |
| | | | | Mol Sci. 17: 2149 |
| | | | | (2016) |
| cTnT | Chicken cardiac | Heart | Cardiomyocytes | Am J Physiol Cell |
| | troponin T | | | Physiol. 286: C556 |
| | promoter | | | (2004); Gene Ther. |
| | | | | 18: 43 (2011); Circ |
| | | | | Res. 115: 354 |
| | | | | (2014) |
| αMHC | Mouse α-cardiac | Heart | Cardiomyocytes | J Biol Chem. |
| (long) | myosin heavy | | | 266: 24613 (1991) |
| | chain promoter | | | |
| | (5.4 kb) | | | |
| αMHC | Mouse α-cardiac | Heart | Cardiomyocytes | J Biol Chem. |
| (short) | myosin heavy | | | 266: 24613 (1991) |
| | chain promoter | | | |
| | (2.8 kb) | | | |
| Myog | Mouse myogenin | Muscle | Myoblasts | Genes Dev. 7: 1277 |
| | promoter | | | (1993) |
| ACTA1 | Human skeletal | Muscle | Myocytes | J Biol Chem. |
| | muscle actin α1 | | | 268: 719 (1993) |
| | promoter | | | |
| MHCK7 | Composed of | Mouse | Differentiated | Mol Ther. 15: 320 |
| | enhancer/promoter | skeletal and | postmitotic striated | (2007); Sci Transl |
| | regions of murine | heart muscles | muscle cells | Med. 9(418) (2017) |
| | muscle creatine | | | |
| | kinase (MCK) and | | | |
| | enhancer region | | | |
| | of α-myosin | | | |
| | heavy-chain genes | | | |
| SM22a | Mouse transgelin | Adult smooth | Vascular smooth | J Cell Biol. |
| | (a.k.a. Sm22a) | muscle | muscle cells | 132: 849 (1996) |
| | promoter | | | |
| EnSM22a | The chimeric | Adult smooth | Vascular smooth | J Pharmacol Exp |
| | vascular smooth | muscle | muscle cells | Ther. 329: 775 |
| | muscle-specific | | | (2009) |
| | enhancer/promoter | | | |
| | (The rabbit myosin | | | |
| | heavy-chain enhancer | | | |
| | inserted upstream | | | |
| | of a truncated | | | |
| | mouse SM22a | | | |
| | promoter) | | | |
| Runx2 | Mouse runt related | Bone | Early osteoblasts | Biochim Biophys |
| | transcription | | | Acta. 1731: 95 |
| | factor 2 promoter | | | (2005) |
| OC | Human osteocalcin | Bone and | Osteoblasts, osteocytes | Mol Endocrinol. |
| | promoter | cartilage | and hypertrophic | 11: 1695 (1997) |
| | | | chondrocytes | |
| Col1a1 | Rat collagen type | Bone, tooth | Osteoblasts and | J Bone Miner Res. |
| | I α1 promoter | and tendon | fibroblasts | 9: 285 (1994) |
| Col2a1 | Mouse collagen type | Cartilage | Chondrocytes | Genesis. 45: 44 |
| | II α1 promoter | | | (2007) |
| aP2 | Rat adipocyte P2 | Fat | Adipocytes | J Biol Chem. |
| | promoter | | | 268: 22243 (1993) |
| Adipoq | Mouse adiponectin | Fat | Adipocytes | Endocrinology. |
| | promoter | | | 151: 2933 (2010) |
| Tie1 | Mouse tyrosine | Blood vessel | Endothelial cells | J Cell Sci. |
| | kinase with | | | 115: 2075 (2002) |
| | immunoglobulin- | | | |
| | like and EGF-like | | | |
| | domains 1 promoter | | | |
| Cd144 | Mouse cadherin 5 | Blood vessels | Vascular endothelial | Dev Dyn. 235:759 |
| | (a.k.a. VE-Cadherin) | | cells | (2006) |
| | promoter | | | |
| CD68 | Human CD68 | Blood | Monocytes and | Gene Ther. |
| (short) | promoter, short, | | macrophages | 19: 1041 (2012) |
| | recommended for | | | |
| | in vitro use | | | |
| CD68 | Human CD68 | Blood | Monocytes and | J Immunol. |
| (long) | promoter, long, | | macrophages | 168: 3402(2002); |
| | recommended for | | | Blood. 124: e33 |
| | in vivo use | | | (2014) |
| CD11b | Human integrin | Bone marrow | Mature myeloid cell | Blood. 79: 865 |
| (also | subunit alpha M | | lines (neutrophils, | (1992); Blood. |
| called | promoter | | monocytes and | 85: 319 (1995) |
| Itgam) | | | macrophages) | |
| Afp | Mouse α-fetoprotein | Liver | Hepatocytes | Science. 235: 53 |
| | enhancer II fused | | | (1987); Mol Cell |
| | with human beta | | | Biol. 15: 4947 |
| | globin promoter | | | (1995) |
| Alb | Mouse albumin | Liver | Mature hepatocytes | Genes Dev. 1: 268 |
| | promoter | | | (1987) |
| TBG | Human | Liver | Hepatocytes | Gene. 506: 289 |
| | thyroxine-binding | | | (2012) |
| | globulin promoter | | | |
| MMTV | Mouse mammary | Mammary gland | Ductal cells of the | Nucleic Acids Res. |
| | tumor virus long | | salivary gland, mammary | 25: 4323 (1997) |
| | terminal repeat | | epithelial cells | |
| | (LTR) | | | |
| Wap | Mouse whey | Mammary gland | Alveolar epithelial | Nucleic Acids Res. |
| | acidic protein | | cells of mammary tissue | 25: 4323 (1997) |
| | promoter | | | |
| HIP | Human insulin | Pancreas | β cells | Cell. 52: 773 |
| | promoter | | | (1998); Proc Natl |
| | | | | Acad Sci USA. |
| | | | | 100: 6688 (2003) |
| Pdx1 | Mouse pancreatic | Embryonic | Pancreatic progenitor | Development. |
| | and duodenal | developing | cells and pancreatic β | 129:2447 (2002) |
| | homeobox 1 | pancreas and | cells | |
| | promoter | adult pancreatic | | |
| | | islets | | |
| Ins2 | Rat insulin 2 | Pancreas | Pancreatic β cells | Nature. 315: 115 |
| | promoter | | | (1985); Nature. |
| | | | | 429: 41 (2004) |
| Hcn4 | Mouse | Embryonic | Cardiomyocytes | Biochem Biophys |
| | hyperpolarization- | heart | | Res Commun. |
| | activated cyclic | | | 353: 67 (2007) |
| | nucleotide-gated | | | |
| | K + 4 promoter | | | |
| NPHS2 | Human podocin | Kidney | Podocytes | Genesis. 35: 39 |
| | promoter | | | (2003) |
| SPB | Human surfactant | Lung | AT II cells (alveolar | J Biol Chem. |
| | protein B promoter | | type II epithelial | 270: 24852 |
| | | | cells) and Clara cells | (1995); Gene Ther. |
| | | | (bronchiolar epithelial | 14: 1461 (2007) |
| | | | cells) | |
| CD144 | Human cadherin 5 | Lung, heart, | Vascular endothelial | Oncogene. |
| | (a.k.a. VE-Cadherin) | ovary, spleen | cells | 24: 2992 (2005) |
| | promoter | and kidney | | |
| | | glomeruli | | |
| TERT | Human telomerase | Tumor | Immortalized and | Cancer Res. 59: 551 |
| | reverse | | cancer cells | (1999); Cancer |
| | transcriptase | | | Res. 60: 5359 |
| | promoter | | | (2000) |

Cells comprising the recombinant rabies viruses described herein are also provided herein.

### Transcriptomics

The methods for tracing cell networks, described herein, can leverage transcriptomic (i.e., RNA) profiling (e.g., single cell transcriptomics). Thus, the methods described herein can comprise, e.g., cell imaging (e.g., to detect fluorescent reporter proteins), cell and/or transcript capture (e.g., cell or tissue isolation and/or permeabilization, e.g., based on detection of the reporter protein), and transcriptome sequencing (e.g., single cell sequencing).

Many different methods for transcriptomic profiling (transcriptome sequencing) are known in the art. See, e.g., Kulkarni et al., “Beyond Bulk: A Review of Single Cell Transcriptomics Methodologies and Applications,” Curr. Opin. Biotechnol. 58:129-36 (2019).

In some embodiments, the method incorporates spatial information, e.g., by visually labeling individual gene markers, e.g., through in situ hybridization (e.g., fluorescent in situ hybridization (FISH). Many variants of this method are known in the art. See Asp et al., “Spatially Resolved Transcriptomes—Next Generation Tools for Tissue Exploration,” Bioessays 42:1900221 (2020) for a discussion of various spatial transcriptomics methods; see also Kulkarni et al., “Beyond Bulk: A Review of Single Cell Transcriptomics Methodologies and Applications,” Curr. Opin. Biotechnol. 58:129-36 (2019); Chen et al., “Spatially Resolved, Highly Multiplexed RNA Profiling in Single Cells,” Science 348(6233):aaa6090-1 (2015); and Moffit et al., “Molecular, Spatial, and Functional Single-Cell Profiling of the Hypothalamic Preoptic Region,” Science 362:eaau5324 (2018).

In some embodiments, the methods described herein comprise single cell transcriptomics, e.g., the inDrops method described herein. Thus, in some embodiments, the methods described herein comprise cell capture, e.g., by flow cytometry. In some embodiments, the methods described herein further comprise isolating mRNA from captured cell(s).

In some embodiments, the methods described herein comprise spatial transcriptomics.

In some embodiments, the methods described herein comprise combined single-cell and spatial transcriptomics methods. See, e.g., Baccin et al., “Combined Single-Cell and Spatial Transcriptomics Reveal the Molecular, Cellular, and Spatial Bone Marrow Niche Organization,” Nat Cell Biol 22(1):38-48 (2020).

### Library Preparation and Sequencing

In some embodiments, the methods described herein comprise adding one or more adapter(s) to mRNA transcripts, e.g., mRNA transcripts of cells comprising the recombinant rabies viruses described herein, captured using any of the methods described herein, to prepare an mRNA library.

In some embodiments, one or more of the adapter(s) comprise priming sites.

In some embodiments, one or more of the adapter(s) comprise a template switching oligonucleotide (TSO).

In some embodiments, one or more of the adapter(s) comprise a barcode that is shared amongst members of the library, e.g., a cell-specific barcode (for example, in the case of single cell transcriptomics methods) or a location-specific barcode (for example, in the case of spatial transcriptomics methods), and combinations thereof.

In some embodiments, one or more of the adapter(s) comprise a unique molecular identifier, e.g., a random sequence that differs amongst adapters within a library.

In some embodiments, the methods described herein comprise amplifying mRNAs, e.g., mRNAs captured from cell(s) comprising the recombinant rabies viruses described herein, including, but not limited to, e.g., mRNAs comprising the reporter genes and barcodes described herein.

Many methods for encoding mRNA library members with, e.g., barcodes and UMIs are known in the art. See, e.g., Zhou et al., “Encoding Method of Single-Cell Spatial Transcriptomics Sequencing,” Int. J. Biol. Sci. 16(14):1663-75 (2020).

In some embodiments, the methods described herein comprise whole transcriptome amplification.

In some embodiments, the methods described herein comprise sequencing, e.g., by next generation sequencing and/or third-generation (long read) sequencing. These sequencing methods are well known and described in the art. See, e.g., van Dijk et al., “The Third Revolution in Sequencing Technology,” Trends Genet. 34(9):666-681 (2018).

In some embodiments, the methods described herein comprise identifying RabΔG barcodes in the mRNA library. In some embodiments, identifying RabΔG barcodes in the mRNA library comprises amplifying, e.g., by PCR, the RabΔG barcodes in the mRNA library. In some embodiments, identifying RabΔG barcodes in the mRNA library comprises sequencing the RabΔG barcodes in the mRNA library.

In some embodiments, the methods described herein comprise network analysis, e.g., transcriptome analysis, connectome reconstruction, and/or interactome discovery; genetic perturbation network analysis within discrete subnetworks since we can theoretically deliver shRNA or sgRNA to affect gene function; analysis of activity within networks by comparing transcriptional activation of immediate early genes alongside actuator expression and activation; activation or inhibition of transcription using dCas9 variants alongside sgRNA expression; epigenetic readout (ATACseq, methylation, ChIP-seq) or even proteomic readout (Abseq); in situ transcriptomics (eg Merfish) as a readout to identify not only interacting cells, but also to determine their location in the CNS or tissue of interest; and a perturb seq approach to perform in vivo screens of genes that affect cell-cell interactions.

### EXAMPLES

The invention is further described in the following examples, which do not limit the scope of the invention described in the claims.

Materials and Methods for Examples 1-3

Mice. B6.Cg-Tg(Gfap-cre)73.12Mvs/J hemizygous mice (The Jackson Laboratory, #012886) were crossed to homozygous B6; 129P2-Gt(ROSA)26Sortm1(CAG-RABVgp4,-TVA)Arenk/J mice (The Jackson Laboratory, #024708). Male mice were used in experiments at >8 weeks of age. All procedures were reviewed and approved by the Brigham and Women's Hospital IACUC.

Molecular biology. First, a cDNA encoding the mCherry ORF was inserted into the vector, pSADAG-GFP-F2 (Addgene, #32635) (SI). PCR with mCherry_FWD and mCherry REV primers in Table 1 on a template plasmid encoding mCherry (Addgene, #80139) (S2) was performed, followed by SbfI and SacII restriction and ligation, which inserted a unique NheI site. A 156 bp dsDNA fragment (Genewiz) was cloned into the NheI/SacII site via XbaI/SacII, which introduced unique NheI/AscI sites flanking a unique PacI site to create pSADΔG-mCherry. Enzymes used in this study were: XbaI (NEB, #R0145S), SacII (NEB, #R0157S), NheI-HF (NEB, #R3131M), AscI (NEB, #R0558S), SbfI-HF (NEB, #R3642L), DpnI (NEB, #R0176L), and PacI (NEB, #R0547L).

TABLE 1
Sequences used to generate and analyze the
barcoded pSADΔG-mCherry plasmid.

| NAME | Sequence | SEQ ID NO: |
|---|---|---|
| mCherry-BC | gccacc ATGGTGAGCAAGGGCGAGGAGGATAACATGG | 81 |
| Code: | CCATCATCAAGGAGTTCATGCGCTTCAAGGTGCACATG | |
| MCHERRY | GAGGGCTCCGTGAACGGCCACGAGTTCGAGATCGAG | |
| PCR handles | GGCGAGGGCGAGGGCCGCCCCTACGAGGGCACCCA | |
| Barcode | GACCGCCAAGCTGAAGGTGACCAAGGGTGGCCCCCT | |
| | GCCCTTCGCCTGGGACATCCTGTCCCCTCAGTTCATG | |
| | TACGGCTCCAAGGCCTACGTGAAGCACCCCGCCGAC | |
| | ATCCCCGACTACTTGAAGCTGTCCTTCCCCGAGGGCT | |
| | TCAAGTGGGAGCGCGTGATGAACTTCGAGGACGGCG | |
| | GCGTGGTGACCGTGACCCAGGACTCCTCCCTGCAGG | |
| | ACGGCGAGTTCATCTACAAGGTGAAGCTGCGCGGCA | |
| | CCAACTTCCCCTCCGACGGCCCCGTAATGCAGAAGA | |
| | AGACCATGGGCTGGGAGGCCTCCTCCGAGCGGATGT | |
| | ACCCCGAGGACGGCGCCCTGAAGGGCGAGATCAAG | |
| | CAGAGGCTGAAGCTGAAGGACGGCGGCCACTACGAC | |
| | GCTGAGGTCAAGACCACCTACAAGGCCAAGAAGCCC | |
| | GTGCAGCTGCCCGGCGCCTACAACGTCAACATCAAG | |
| | TTGGACATCACCTCCCACAACGAGGACTACACCATCG | |
| | TGGAACAGTACGAACGCGCCGAGGGCCGCCACTCCA | |
| | CCGGCGGCATGGACGAGCTGTACAAGTAATAAgctaga | |
| | GATGTCCACGAGGTCTCTgctagcVHDBVHDBATVH | |
| | DBVHDBATVHDBVHDB ggcgcgccCGTACGCTGCAG | |
| | GTCGACccgcggTAGCTTTTCAGTCGAGAAAAAAA | |
| mCherry_FWD | AAACCTGCAGGGCCACCATGGTGAGCAAGGG | 3 |
| primer | | |
| mCherry_REV | TTTCCGCGGTTTTTTGCTAGCTTACTTGTACAGCTC | 4 |
| primer | GTCCATGC | |
| Rabies barcode | AAGTAAGCTAGAGATGTCCACGAGGTCTCTGCTAG | 5 |
| | C(V:33333300)(H:33330033) | |
| | (D:33003333)(B:00333333)V | |
| | HDBATVHDBVHDBATVHDBVHDBGGCGCGCCCGT | |
| | ACGCTGCAGGTCGACCCGCGGTAGC | |
| RAB_P7_C01 | CAAGCAGAAGACGGCATACGAGATGCACGACCGT | 6 |
| | GACTGGAGTTCAGACGTGTGCTCTTCCGATCTGTC | |
| | GACCTGCAGCGTACG | |
| RAB_P5_s0 | AATGATACGGCGACCACCGAGATCTACACTCTTTC | 7 |
| | CCTACACGACGCTCTTCCGATCTGATGTCCACGAG | |
| | GTCTCT | |
| RAB_P5_s1 | AATGATACGGCGACCACCGAGATCTACACTCTTTC | 8 |
| | CCTACACGACGCTCTTCCGATCTCGATGTCCACGA | |
| | GGTCTCT | |
| RAB_P5_s2 | AATGATACGGCGACCACCGAGATCTACACTCTTTC | 9 |
| | CCTACACGACGCTCTTCCGATCTGCGATGTCCACG | |
| | AGGTCTCT | |
| RAB_P5_s3 | AATGATACGGCGACCACCGAGATCTACACTCTTTC | 10 |
| | CCTACACGACGCTCTTCCGATCTAGCGATGTCCAC | |
| | GAGGTCTCT | |
| RAB_P5_s4 | AATGATACGGCGACCACCGAGATCTACACTCTTTC | 11 |
| | CCTACACGACGCTCTTCCGATCTCAACGATGTCCA | |
| | CGAGGTCTCT | |
| RAB_P5_s6 | AATGATACGGCGACCACCGAGATCTACACTCTTTC | 12 |
| | CCTACACGACGCTCTTCCGATCTTGCACCGATGTCC | |
| | ACGAGGTCTCT | |
| RAB_P5_s7 | AATGATACGGCGACCACCGAGATCTACACTCTTTC | 13 |
| | CCTACACGACGCTCTTCCGATCTACGCAACGATGT | |
| | CCACGAGGTCTCT | |
| RAB_P5_s8 | AATGATACGGCGACCACCGAGATCTACACTCTTTC | 14 |
| | CCTACACGACGCTCTTCCGATCTGAAGACCCGATG | |
| | TCCACGAGGTCTCT | |

Rabies library barcode generation. Gibson assembly was used to insert barcodes downstream of the mCherry translational stop codon and before the pseudorabies virus polyA transcriptional stop signal (S3). The barcode VHDBVHDBATVHDBVHDBATVHDBVHDB (SEQ ID NO:1) was synthesized from IDT as a single stranded oligonucleotide with flanking handles to allow for barcode recovery (Table 1). The plasmid pSADΔG-mCherry was linearized with NheI-HF (NEB, #R3131S) and AscI (NEB, #R0558S) for 16 hours at 37 C, mixed at a molar ratio of 5:1 barcode:plasmid, and assembled with NEBuilder HiFi DNA Assembly Master Mix (NEB, #E2621L) according to the manufacturer's instructions. Fifty reactions of 20 μL were pooled after assembly, purified using 2.0× Ampure XP magnetic beads (Beckman-Coulter, #A63881), and resuspended so as to concentrate 25-fold. The purified plasmid was electroporated into ElectroMAX Stb14 Competent Cells (Thermo Fisher, #11635018). Briefly, a 100 μL aliquot of Stb14 was combined with 4 μL of purified assembled plasmid, divided into 25 μL per cuvette and electroporated (Biorad Gene Pulser II; 1.2 kV, 200 Ohm, 25 μF). A total of 40 electroporations were performed. The cells were recovered at 30° C. in Zymo Broth (Zymo Research, #M3015-100) shaken at 225 rpm for 1 hour and plated at 30° C. overnight divided across sixteen 625 cm² agar plates (Thermo Fisher, #240835) with 100 μg/mL ampicillin (Sigma-Aldrich, #A9518-5G). Colonies were scraped into 20 mL LB per plate, shaken at 225 rpm at 30 C for 4 hours, and purified using an endotoxin-free plasmid Giga kit (Qiagen, #12391) resulting in 340 μg of plasmid. Test digestions of the assembled plasmid (RabΔG-mCherry-BC) using PacI (NEB, #R0547L) confirmed undetectable levels of non-linearized plasmid carryover.

Barcode amplification from DNA. Library amplification and sequencing to test for barcode diversity was performed based on similar protocols for genome-wide CRISPR libraries (S4). PCR reactions were set up using Phusion Flash HF PCR master mix (Thermo Fisher, #F548L) with 0.5 M each of forward and reverse primers, 3% DMSO, and 4 ng/μl plasmid DNA per reaction. Reactions were thermocycled at 98 C for 30 s, 18 cycles of [98 C for 10 s, 62 C for 30 s, 72 C for 60 s], followed by 72 C for 5 minutes, and 4 C forever. A staggered forward primer cocktail, made by combining equimolar concentrations of P5 primers (RAB_P5_s0, RAB_P5_s1, RAB_P5_s2, RAB_P5_s3, RAB_P5_s4, RAB_P5_s6, RAB_P5_s7, and RAB_P5_s8) was used with the RAB_P7_C01 primer to amplify the plasmid (Table 1).

Rabies virus production. All plasmids used for rabies virus production were prepared using an endotoxin-free plasmid Giga kit (Qiagen, #12391). Pseudotyped G-deficient rabies virus was produced largely as previously described (S5). Briefly, one day prior to transfection, baby hamster kidney (BHK) cells expressing T7 RNA polymerase, rabies glycoprotein G, and GFP (hereafter: B7GG cells) were seeded into ten 10-cm plates (Thermo Fisher Scientific, #08-772E) at a density of 2.2×10⁶ cells/plate. Cells were grown in DMEM with high glucose, L-glutamine, and sodium pyruvate (Life Technologies, #11995073) supplemented with 10% FBS (Life Technologies, #10438026) (hereafter: B7GG media). The next day, they were transfected with the following helper plasmids: 150 μg pcDNA-SADB19N (Addgene, #32630), 75 μg pcDNA-SADB19P (Addgene, #32631), 75 μg pcDNA-SADB19L (Addgene, #32632), 50 μg pcDNA-SADB19G (Addgene, #32633) (S1), and 300 μg RabΔG-mCherry-BC using Lipofectamine 2000 (Thermo Fisher Scientific, #11668019) according to the standard protocol. Cells were transfected at 37 C and 5% CO2 for 18 hours. The next day, cells were split into 30 15-cm plates (Fisher Scientific, #08-772-6) and grown at 35C and 3% CO2. The following day, the supernatant was aspirated, and 24 mL fresh media was added to each plate. Thereafter, every 2 days, 10 mL of B7GG media was added to each plate. Then 2 days later, all viral supernatant was harvested, vacuum filtered through 0.45 μm pores (Fisher Scientific, #SCHVU11RE) and frozen. Altogether, 6 batches of viral supernatant were collected. For viral pseudotyping, 10 15-cm plates of BHK cells expressing the rabies virus envelope protein, EnvA, (hereafter BHK-EnvA) were seeded at 60% confluency at 35 C and 3% CO2. The next day, unpseudotyped virus was applied to the BHK-EnvA cells for 48 hours. Each dish was washed 10× with 1×PBS to remove unpseudotyped virus. The next day, viral supernatant was aspirated and 24 mL of fresh B7GG media was added. Thereafter, viral supernatant was collected every 2 days. Altogether, 5 pseudotyped viral preparations were collected. To concentrate pseudotyped and unpseudotyped RabΔG-mCherry-BC viruses, 35 mL of the collected supernatant was ultracentrifuged using a Beckman SW28 rotor at 70,000 g for 2 hours at 4 C. Afterwards, the pellets were resuspended in HBSS, gently vortexed briefly, and were added to 2.5 mL of 20% sucrose in HBSS. Virus was centrifuged at 50,000 g for 2 hours at 4C using am SW55 rotor. Each viral pellet was resuspended in 100 μL ice cold HBSS, briefly vortexed, and chilled at 4 C for >1 hour. Viral titration and pseudotyping specificity were performed using HEK293-TVA cells and HEK293 cells as described (S5). The B7GG, BHK-EnvA, and HEK293-TVA cell lines were obtained from the GT3 Core Facility of the Salk Institute with funding from NIH-NCI CCSG: P30 014195, an NINDS R24 Core Grant and funding from NEI.

Rabies virus diversity analysis. To analyze rabies virus diversity, five 90% confluent 15-cm plates of HEK293T cells were transduced with rabies virus supernatant in vitro for 24 hours. 7 days post-transduction, RNA was harvested from cells and processed using an RNeasy Maxi kit (Qiagen, #75162). 5 μg RNA was reverse transcribed using 0.2 μL 100 μM Oligo(dT)20 primer (SEQ ID NO: 82), 1 μL 10 mM dNTP (Thermo Fisher Scientific, #R0191), 4 μL RT buffer, 0.5 μL RNase inhibitor (Lucigen), 1 μL Maxima H Minus (Thermo Scientific, #EP0752) and filled to 20 μL with nuclease free H2O. RT was performed at 50 C for 30 minutes, followed by 85 C for 5 minutes, then 4 C forever. cDNA was purified using Ampure RNAclean beads at a ratio of 2.0×. PCR of pSADAG- mCherry-BC plasmid of reverse transcribed cDNA was used for barcode amplification and addition of Illumina adapters.

EAE. EAE was performed as previously described (S6, S7). EAE was induced with 200 μg of MOG35-55 (Genemed Synthesis Inc., #110582,) mixed with freshly prepared complete Freund's Adjuvant (using 20 mL Incomplete Freund's Adjuvant (BD Biosciences, #BD263910) mixed with 100 mg M. Tuberculosis H-37Ra (BD Biosciences, #231141)) at a ratio of 1:1 (v/v at a concentration of 5 mg/mL). Mice received 2 subcutaneous injections of 100 μL each of the MOG/CFA mix. Mice then received a single intraperitoneal injection of pertussis toxin (List Biological Laboratories, #180) at a concentration of 2 ng/μL in 200 μL of PBS. Mice received a second injection of pertussis toxin at the same concentration two days after the initial EAE induction. Mice were monitored and scored daily thereafter. EAE clinical scores were defined as follows: 0—no signs, 1—fully limp tail, 2—hindlimb weakness, 3—hindlimb paralysis, 4—forelimb paralysis, 5—moribund, as described previously (S6-S10).

RabΔG viral transduction. Intracranial delivery of RabΔG was performed largely as described previously (S6, S7). Briefly, mice were anesthetized using 1-3% isoflurane mixed with oxygen. Heads were shaved and cleaned using 70% ethanol and Betadine (Thermo Fisher, #19-027132) followed by a medial incision of the skin to expose the skull. The forebrain was targeted unilaterally using the coordinates: +1.8 (lateral), +0.5 (rostral), −3.0 (ventral) relative to Bregma. Mice were injected with a RabΔG viral dilution in 1 μL capable of seeding 1,000 cells using a 5 μL Hamilton syringe on a Stereotaxic Alignment System (Kopf, #1900), sutured, and permitted to recover in a separate clean cage. Mice were sacrificed 7.5 days post-injection for RABID-seq experiments.

Isolation of cells from the mouse CNS. Cells were isolated by flow cytometry as described (S6-S9) with modifications. Briefly, mice were perfused with 1×PBS and the transduced CNS region (approx. 27 mm ³) was isolated into 10 mL of enzyme digestion solution consisting of 75 μL Papain suspension (Worthington, #LS003126) diluted in enzyme stock solution (ESS) and equilibrated to 37 C. ESS consisted of 10 mL 10×EBSS (Sigma-Aldrich, #E7510), 2.4 mL 30% D(+)-Glucose (Sigma-Aldrich, #G8769), 5.2 mL 1M NaHCO3 (VWR, #AAJ62495-AP), 200 μL 500 mM EDTA (Thermo Fisher Scientific, #15575020), and 168.2 mL ddH2O, filter-sterilized through a 0.22 μm filter. Samples were shaken at 80 rpm for 30 minutes at 37 C. Enzymatic digestion was stopped by adding 1 mL of 10×hi ovomucoid inhibitor solution and 20 μL 0.4% DNase (Worthington, #LS002007) diluted in 10 mL inhibitor stock solution (ISS). 10×hi ovomucoid inhibitor stock solution contained 300 mg BSA (Sigma-Aldrich, #A8806), 300 mg ovomucoid trypsin inhibitor (Worthington, #LS003086) diluted in 10 mL 1×PBS and filter sterilized using at 0.22 μm filter. ISS contained 50 mL 10×EB SS (Sigma-Aldrich, #E7510), 6 mL 30% D(+)-Glucose (Sigma- Aldrich, #G8769), 13 mL 1M NaHCO3 (VWR, #AAJ62495-AP) diluted in 170.4 mL ddH2O and filter-sterilized through a 0.22 μm filter. Tissue was gently mechanically dissociated using a 5 mL serological pipette and filtered through at 70 μm cell strainer (Fisher Scientific, #22363548) into a fresh 50 mL conical. Dead cells and myelin were removed from the cell pellet using the Miltenyi MACS isolation kits (Myelin: #130-096-733; Dead cells: #130-090-101). LS columns and the Annexin V binding buffer were used for wash steps and elution. The cell pellet (¼ to ⅛ mass of whole brain) was resuspended in 150 of Myelin removal beads and 150 μL of dead cell removal beads and incubated at room temperature for 15 minutes. Cells were then processed according to the manufacturer's protocol. Eluate was collected and centrifuged at 600 g for 5 minutes. Cells were resuspended in of FACS buffer (500 μM EDTA, 1% BSA, 0.9×PBS) on ice until sorting. Flow cytometry. Cells were filtered through a 35 μm filter prior to sorting (Fisher

Scientific, #08-771-23). Cells were sorted on a FACS Aria IIu (BD Biosciences). Gating parameters were established using a WT control during each batch of flow cytometry. Cells were gated as intact, followed by exclusion of FSC and SSC doublets. Sorting of mCherry+ cells was judged in the PE-Texas Red channel using a yellow-green laser. Cells were sorted at low flow rates (1-2) through a 100 μm nozzle using purity settings. Approximately >90% of all labeled mCherry+cells were sorted across all mice.

InDrop single cell RNA-sequencing. Cells were resuspended at a concentration of 100,000 cells/mL in PBS with 0.1% BSA, 18% Optiprep and 0.1% Pluronic F-68 (Thermo Fisher, #24040032). Microfluidic cell encapsulation with barcoded beads was performed using the microfluidic device and flow rates previously described (S11). FACS sorted mCherry+ cells were co-flowed with reverse transcription reagents at equal flow rates. Barcoded beads were obtained from the Harvard Single Cell Core (inDrop v3). Modifications to the molecular biology were made to enable reverse transcription with template switching in drops. A 2× reverse transcription master mix was prepared at a final concentration of 1 mM dNTP (NEB, #N04475), 50 μM TSO oligonucleotide (Table 2), 0.6% v/v IGEPAL CA-630 (Sigma-Aldrich, #56741), 2×RT Buffer (Thermo Scientific, #EP0751), 1 U/μl NxGen RNase Inhibitor (Lucigen, #30281-2), and 20 U/μl Maxima H minus RT (Thermo Scientific, #EP0751). Drops were collected on ice in batches of 3000 cells, and immediately UV treated for exactly 8 minutes (Analytik Jena Blak-Ray XX-15L UV light source) to release primers. A mineral oil overlay (Sigma-Aldrich, #M5310-1L) was placed on the emulsion and reverse transcription was performed for 60 minutes at 42 C in a heat block. Droplets were incubated at 5 minutes at 85° C. to inactivate the reverse transcriptase and the mineral oil overlay was carefully removed with a P1000 pipet. The bottom layer of oil was removed, and the emulsion was broken by the addition of 5X v/v 20% 1H,1H,2H,2H-Perfluoro-1-octanol (Sigma-Aldrich, #370533-25G) in oil (3M, HFE-7500 Novec Engineered fluid). The aqueous phase was transferred onto a spin filter

(Corning, #8162) to remove the barcoded beads, purified with 2.0×RNAclean XP (Beckman Coulter, #A63987), and eluted in 20 μL of ddH2O. Whole transcriptome amplification (WTA) was performed using 20 μL of purified cDNA, 0.5 μM inDrops_FWD primer (Table 2), 0.5 μM inDrops_REV primer (Table 2), and 1× KAPA HiFi master mix (KAPA Biosystems #KK2601) in a 50 μL reaction. The PCR program used was 98 C for 3 minutes, followed by 15 cycles of: [98 C for 15 s, 67 C for 20 s, 72 C for 3 min] then 72 C for 5 min, followed by 4 C forever. After amplification, WTA product was purified with 0.6×AMPure XP beads, diluted, and analyzed using a Bioanalyzer DNA HS assay (Agilent, #50674626). Single cell RNA-seq libraries were prepared using a NEBNext Ultra II FS Kit (NEB, #E7805) using a custom pre-annealed adapter at the concentrations recommended by the manufacturer, in accordance with the concentration of WTA product. The adapter was prepared by mixing 100 μM Ligation_FWD oligonucleotide and 100 μM Ligation REV oligonucleotide (Table 2) and heating to 95 C for 2 minutes, followed by cooling to room temperature for 5 minutes. The annealed adapter was suspended to 1.5 μM, aliquoted, and stored at −20° C. until use. DNA was suspended to 50 ng of material and fragmented using the NEB kit, followed by end repair, and adaptor ligation, all according to the manufacturer's protocol. Sequencing libraries were amplified from purified WTA product using 10 μM of inDropV3_P5_r1_S5XX primer (Table 3) and 10 μM of inDropV3_P7_r2 primer (Table S3) in NEBNext Q5 PCR master mix. Samples were cycled at 98 C for 45 s, followed by 14 cycles of: [98 C for 20 s, 54 C for 30 s, 72 C for 20 s], and 72 C for 60 s, then 4C forever. Samples were purified by 0.8× Ampure XP bead purification and eluted in 21 μL ddH2O. Libraries were quantified by Bioanalyzer and Kapa Library Quantification Kit (Kapa Biosystems, #KK4824) prior to sequencing on a NextSeq550.

TABLE 2
Primers used for the SMART-seq based
inDrop approach (5′ to 3′). XX
Indicates S5XX index sequence used.

| | | SEQ |
|---|---|---|
| | | ID |
| Primer | Sequence | NO: |
| TSO | AAGCAGTGGTATCAAC | 15 |
| | GCAGAGTACATrGrGrG | |
| inDrops_FWD | GGGTGTCGGGTGCAG | 16 |
| inDrops_REV | AAGCAGTGGTATCAACG | 17 |
| | CAGAGTACAT | |
| Ligation_FWD | CTGTCTCTTATACACAT | 18 |
| | CTGACGCTGCCGACGA | |
| Ligation_REV | AGATGTGTATAAGAGAC | 19 |
| | AGT | |
| PCR_p5 r1_ | AATGATACGGCGACCAC | 20 |
| S5XX | CGAGATCTACACXXXX | |
| | XXXXTCGTCGGCAGCGTC | |
| inDrop_v3_ | CAAGCAGAAGACGGCATA | 21 |
| p7_r2 | CGAGATGGGTGTCGG | |
| | GTGCAG | |

TABLE 3
Primers used for RabAG barcode amplification
from WTA product (5′ to 3′).
XXXXXXXX: Indicates S5XX index sequence used.

| | | SEQ |
|---|---|---|
| | | ID |
| Primer | Sequence | NO: |
| SMART mCherry | TACACCATCGTGGAACAGTACGAAC | 22 |
| primer | | |
| inDrop_FWD | GGGTGTCGGGTGCAG | 23 |
| inDrop_v3_ | CAAGCAGAAGACGGCATACGAGATGGGT | 24 |
| p7_r2 | GTCGGGTGCAG | |
| inDropv3_ | AATGATACGGCGACCACCGAGATCTACA | 25 |
| S5XX_R2bc | CXXXXXXXXTCGTCGGCAGCGTCAGATG | |
| | TGTATAAGAGACAGGATGTCCACGAGGT | |
| | CTCT | |
| inDropv3_ | AATGATACGGCGACCACCGAGATCTACAC | 26 |
| S5XX_R2bc+1 | XXXXXXXXTCGTCGGCAGCGTCAGATGTG | |
| | TATAAGAGACAGaGATGTCC | |
| | ACGAGGTCTCT | |
| inDropv3_ | AATGATACGGCGACCACCGAGATCTACA | 27 |
| S5XX_R2bc+2 | CXXXXXXXXTCGTCGGCAGCGTCAGATG | |
| | TGTATAAGAGACAGctGATGTCC | |
| | ACGAGGTCTCT | |
| inDropv3_ | AATGATACGGCGACCACCGAGATCTACAC | 28 |
| S5XX_R2bc+3 | XXXXXXXXTCGTCGGCAGCGTCAGATGTG | |
| | TATAAGAGACAGtccGATGTC | |
| | CACGAGGTCTCT | |

### Primer Conditions for Rabies Barcode Amplification:

Round 1:
   10 μM FWD
   10 μM SMART mCherry
Round 2:
   10 μM inDrop v3_p7_r2
   10 μM inDropinDropv3_S5XX _StaggerMix:
      inDropv3_S5XX_R2bc
      inDropv3_S5XX_R2bc+1
      inDropv3_S5XX_R2bc+2
      inDropv3_S5XX_R2bc+3

Rabies barcode recovery from in vivo experiments. To isolate barcodes from cDNA libraries generated from mCherry+ cells, we derived an approach based on the ATAC-seq protocol (S12, S13). First, 1-25 nM of whole transcriptome amplified cDNA (WTA product) was

PCRed using the following: 2.5 μL of 10 μM inDrops_FWD cDNA amplification primer (Table 3), 2.5 μL of 10 μM SMART mCherry primer (Table S3), 25 μL of NEBNext High-Fidelity 2× PCR Master Mix (NEB, #M0541L) and molecular grade water to fill a 50 μL reaction under the cycling conditions: 98 C for 30 sec, 5 cycles of [98 C for 20 s, 63 C for 30 s, 72 C for 10 sec], and 4 C forever. Following PCR, 5 μL of each sample was analyzed by qPCR in a master mix consisting of 4.4 μL H2O, 0.25 μL 0.5 μM inDrop FWD, 0.25 μL 0.5 μM SMART mCherry, 0.09 μL 100×SYBR Green I (Thermo Fisher, #S7563), and 5 μL NEBNext High-Fidelity 2×PCR Master Mix to determine the relative amount of DNA in the sample; the remainder of the sample was stored on ice. Samples were cycled by qPCR using the following conditions: 98 C for 30 s and 30 cycles of [98 C for 20 s, 63 C for 30 s, 72 C for 10 s]. The number of cycles (N) required to achieve ⅓ the maximal fluorescence was calculated for each sample and the remaining 45 of sample was cycled using the following conditions: 98 C for 30 s, and N cycles of [98 C for 20 s, 63 C for 30 s, 72 C for 10 s], and 4 C forever. Samples were then purified using Ampure XP magnetic beads (Beckman-Coulter, #A63881) using double-sided bead purification according to the manufacturer's protocol. First, large DNA fragments were removed using a 0.7× beads:volume ratio (31.5 μL beads). Supernatant was collected and incubated with 1.0× bead:volume (76.5 μL) purification to eliminate primer dimers. DNA was resuspended in 30 of DNase/RNase-free water. Next, DNA samples were PCRed using the following conditions: 23 μL purified PCR product from round 1, 25 μL NEBNext High-Fidelity 2×PCR Master Mix, 1 μL 10 μM inDrop_v3_p7_r2 (Table 3) and 1 μL of 10 μM staggered cocktail, which contained equimolar concentrations of inDropv3_S5XX_R2bc, inDropv3_S5XX_R2bc+1, inDropv3_S5XX_R2bc+2, inDropv3_S5XX_R2bc+3 (Table 3). Indices from N501-N536 were used (Table 4). Samples were cycled using the following conditions: 98 C for 30 sec and 10 cycles of [98 C for 20 s, 55 C for 20 s, 72 C for 10 s], followed by 72 C for 2 minutes, and 4 C forever. Following PCR, samples were purified using a double-sided Ampure XP bead purification (0.7× (35 μL) followed by 1.0× (85 μL) and resuspended in 15 μL of H2O. Samples were then quantified on a 2100 Bioanalzyer (Agilent Technologies). Samples were quantified by qPCR prior to deep sequencing using the Kapa Library Quantification Kit (Kapa Biosystems, #KK4824).

TABLE 4
Index sequences used (5′ to 3′).

| Primer name | Index | SEQ ID NO: |
|---|---|---|
| inDropv3_S501_R2bc | TAGATCGC | 29 |
| inDropv3_S502_R2bc | CTCTCTAT | 30 |
| inDropv3_S503_R2bc | TATCCTCT | 31 |
| inDropv3_S504_R2bc | AGAGTAGA | 32 |
| inDropv3_S505_R2bc | GTAAGGAG | 33 |
| inDropv3_S506_R2bc | ACTGCATA | 34 |
| inDropv3_S507_R2bc | AAGGAGTA | 35 |
| inDropv3_S508_R2bc | CTAAGCCT | 36 |
| inDropv3_S509_R2bc | GGCTACTC | 37 |
| inDropv3_S510_R2bc | CCTCAGAC | 38 |
| inDropv3_S511_R2bc | TCCTTACG | 39 |
| inDropv3_S512_R2bc | ACGCGTGG | 40 |
| inDropv3_S513_R2bc | GGAACTCC | 41 |
| inDropv3_S514_R2bc | TGGCCATG | 42 |
| inDropv3_S515_R2bc | GAGAGATT | 43 |
| inDropv3_S516_R2bc | CGCGGTTA | 44 |
| inDropv3_S517_R2bc | GACCGCCA | 45 |
| inDropv3_S518_R2bc | TAAGATGG | 46 |
| inDropv3_S519_R2bc | ATTGACAT | 47 |
| inDropv3_S520_R2bc | AGCCAACT | 48 |
| inDropv3_S521_R2bc | TACTAGGT | 49 |
| inDropv3_S522_R2bc | TCACGGTT | 50 |
| inDropv3_S523_R2bc | TGTAATGA | 51 |
| inDropv3_S524_R2bc | CACGTCAG | 52 |
| inDropv3_S525_R2bc | CTGAATTC | 53 |
| inDropv3_S526_R2bc | CGTACCGG | 54 |
| inDropv3_S527_R2bc | GATGACGG | 55 |
| inDropv3_S528_R2bc | TATAGACG | 56 |
| inDropv3_S529_R2bc | GTCATTGA | 57 |
| inDropv3_S530_R2bc | GCATCGTT | 58 |
| inDropv3_S531_R2bc | AGGTTGAC | 59 |
| inDropv3_S532_R2bc | TGAAACTG | 60 |
| inDropv3_S533_R2bc | CAATCACA | 61 |
| inDropv3_S534_R2bc | ACATGCAA | 62 |
| inDropv3_S535_R2bc | ATCGCGCC | 63 |
| inDropv3_S536_R2bc | TCGGTTAA | 64 |

Lentivirus production and transduction. Lentiviral constructs were generated by modifying the pLenti-U6-sgScramble-Gfap-Cas9-2A-EGFP-WPRE lentiviral backbone, described previously (S6, S7). This backbone contains derivatives of the previously described reagents lentiCRISPR v2 (Addgene plasmid #52961 (S14)), and lentiCas9-EGFP (Addgene plasmid #63592 (S15)). The Gfap promoter is the ABC1D gfa2 GFAP promoter (S16). The Itgam promoter (also known as Cd11b) we described previously (S10). Substitution of sgRNAs was performed through a PCR-based cloning strategy using Phusion Flash HF 2× Master Mix (Thermo Fisher, #F548L). A three-way cloning strategy was developed to substitute sgRNAs using the following primers: U6-PCR-F and U6-PCR-R; cr-RNA-F and cr-RNA-R′ (Table 5). Amplicons were purified using the QIAquick PCR Purification Kit (Qiagen, #28104) and digested using DpnI (NEB, #R0176S), BsaI-HF (NEB, #R3535/R3733), AscII (for U6 fragment) (NEB, #R0558), or SbfI-HF (for crRNA fragment) (NEB, #R3642). pLenti backbone was cut with AscI/SbfI-HF and purified using the QIAquick PCR purification kit. Ligations were performed overnight at 16 C using T4 DNA Ligase Kit (NEB, #M0202L). Ligations were transformed into NEB Stable Cells (NEB, #C3040) at 37 C, single colonies were picked, and DNA was prepared using QIAprep Spin Miniprep Kit (Qiagen, #27104). Lentiviral plasmids were transfected into HEK293FT cells according to the ViraPower Lentiviral Packaging Mix protocol (Thermo Fisher Scientific, #K497500) and lentiviruses were packaged with pLP1, pLP2, and pseudotyped with pLP/VSVG. Media was changed the next day, lentivirus was collected 48 hours later and concentrated using Lenti-X Concentrator (Clontech. #631231) overnight at 4 C followed by centrifugation according to the manufacturer's protocol and resuspension in 1/100- 1/500 of the original volume in 1×PBS. Delivery of lentiviruses via intracerebroventricular (ICV) injection was performed largely as described previously (S6, S7). Briefly, mice were anesthetized using 1-3% isoflurane mixed with oxygen. Heads were shaved and cleaned using 70% ethanol and Betadine (Thermo Fisher, #19-027132) followed by a medial incision of the skin to expose the skull. The ventricles were targeted bilaterally using the coordinates: +/−1.0 (lateral), −0.44 (posterior), −2.2 (ventral) relative to Bregma. Mice were injected with approximately 10⁷ total IU of lentivirus delivered by two 10 μL injections using a 25 μL Hamilton syringe (Sigma-Aldrich, #20787) on a stereotaxic alignment system (Kopf, #1900), sutured, and permitted to recover in a separate clean cage. Mice were permitted to recover for between 4-7 days before induction of EAE. CRISPR/Cas9 sgRNA sequences were designed using a combination of the Broad Institute's sgRNA GPP Web Portal (portals.broadinstitute.org/gpp/public/analysis-tools/sgrna-design), Synthego (design.synthego.com/#/validate), and cross-referenced with activity-optimized sequences contained within the Addgene library #1000000096 (S17). sgRNAs used in this study were: sgSema4d, sgPlxnb1, sgPlxnb2, and sgScramble (sequence from Origene, #GE100003) (Table 6).

TABLE 5
Primers used for sgRNA plasmid generation
(5′ to 3′), where N20 marks the sgRNA
substitution site.

| | | SEQ |
|---|---|---|
| Primer | | ID |
| name | Index sequence | NO: |
| U6-PCR-F | AAAGGCGCGCCGAGGGCCTATTT | 65 |
| U6-PCR-R | TTTTTTGGTCTCCCGGTGTTTCG | 66 |
| | TCCTTTCCAC | |
| cr-RNA-F | AAAAAAGGTCTCTACCG(N20) | 67 |
| | GTTTTAGAGCTAGAAATAGCAAGTT | |
| cr-RNA-R | GTTCCCTGCAGGAAAAAAGCACCGA | 68 |

TABLE 6
sgRNA sequences used in this study (5′ to 3′).

| | | SEQ |
|---|---|---|
| | | ID |
| sgRNA name | Index sequence | NO: |
| sgSema4d | GCCGAGTAGTTAAAGATGCC | 69 |
| sgPlxnb1 | GGGGAAGGCACAGAGCACAG | 70 |
| sgPlxnb2 | GGAGGTCACCAGCCCCACGG | 71 |
| sgScramble | GCACTACCAGAGCTAACTCA | 72 |

Immunostaining. Mice were intracardially perfused with ice cold 1×PBS followed by ice cold 4% PFA. Brains were harvested, post-fixed in 4% PFA overnight at 4 C, followed by dehydration in 30% sucrose for 2 days at 4 C. Brains were then frozen in OCT (Sakura, #4583) and 30 μm sections were obtained by cryostat on SuperFrost Plus slides (Fisher Scientific, #22-037-246). A hydrophobic barrier was drawn (Vector Laboratories, #H-4000) and sections were washed 3× for 5 minutes with 0.1% Triton X-100 in PBS (PBS-T). Sections were permeabilized with 0.3% PBS- T for 20 minutes, then washed 3× with 0.1% PBS-T. Sections were blocked with 5% donkey serum (Sigma-Aldrich, #D9663) in 0.1% PBS-T at RT for 30 minutes. Sections were then incubated with primary antibodies diluted in blocking buffer overnight at 4 C. Following primary antibody incubation, sections were washed 3× with 0.1% PBS-T and incubated with secondary antibodies diluted in blocking buffer for 2 hours at RT. Following secondary incubation, sections were washed 3× with 0.1% PBS-T, dried, and coverslips were mounted using Fluoromount-G with DAPI (SouthernBiotech, #0100-20). Primary antibodies used in this study were: mouse anti- GFAP (Millipore, 1:500, #MAB360), chicken anti-GFP (Abcam, #ab13970, 1:1000), rabbit anti- mCherry (Abcam, 1:500, ab167453), Armenian hamster anti-PlexinB2 Antibody (Fisher Scientific, 1:100, #5013113), rabbit anti-Iba1 (Abcam, 1:100, ab178846), and rabbit anti-Semaphorin 4D/CD100 (Abeam, 1:100, #ab231961). Secondary antibodies used in this study were: Alexa Fluor 647 donkey anti-mouse (Abeam, #ab150107), donkey anti-Rabbit IgG (H+L) Highly Cross-Adsorbed, Alexa Fluor 568 (Life Technologies, #A10042), Rhodamine Red-X-AffiniPure Fab Fragment Donkey Anti-Rabbit IgG (H+L) (Jackson Immunoresearch, #711-297-003), Rhodamine Red-X-AffiniPure Fab Fragment Donkey Anti-Mouse IgG (H+L) (Jackson Immunoresearch, #715-297-003), Alexa Fluor 488 AffiniPure Fab Fragment Goat Anti-Rabbit IgG (H+L) (Jackson Immunoresearch, #111-547-003), Alexa Fluor 647 AffiniPure Fab Fragment Donkey Anti-Rabbit IgG (H+L) (Jackson Immunoresearch, #711-607-003), Goat anti-Rabbit IgG (H+L) Cross-Adsorbed Secondary Antibody, Alexa Fluor 405 (Thermo Fisher, #A-31556), Goat Anti-Armenian hamster IgG H&L Alexa Fluor 568 (Abeam, #ab175716), Alexa Fluor 488-AffiniPure Donkey Anti-Rabbit IgG (H+L) (Jackson Immunoresearch, #711-545-152), and Goat anti-Chicken IgY (H+L) Alexa Fluor 488 (Life Technologies, #A11039) all at 1:500 working dilution. Iterative labeling using rabbit primary antibodies was accomplished by incubating with a single primary antibody on Day 1, staining with the anti-rabbit Fab fragment on Day 2, washing 6× with PBS-T, followed by incubation with primary and secondary antibodies as described above.

Primary astrocyte and microglia cultures. Procedures were performed largely as described previously (S6, S7, S10). Brains of mice aged P0-P3 were dissected into PBS on ice. Brains were centrifuged at 500 g for 10 minutes at 4 C and resuspended in 0.25% Trypsin-EDTA (Thermo Fisher Scientific, #25200-072) at 37C for 10 minutes. DNase I (Thermo Fisher

Scientific, #90083) was then added at a concentration of 1 mg/mL to the solution, and the brains were digested for 10 more minutes at 37 C. Trypsin was neutralized by adding DMEM/F12+GlutaMAX (Thermo Fisher Scientific, #10565018) supplemented with 10% FBS (Thermo Fisher Scientific, #10438026) and 1% penicillin/streptomycin (Thermo Fisher Scientific, #15140148), and cells were passed through a 70 μm cell strainer. Cells were centrifuged at 500 g for 10 minutes at 4° C., resuspended in DMEM/F12+GlutaMAX with 10% FBS/1% penicillin/streptomycin and cultured in T-75 flasks (Falcon, #353136) pre-coated with Poly-L-lysine (Sigma Aldrich, #P4707) for 1 h at 37 C and washed with 1×PBS. Cells were cultured at 37 C in a humidified incubator with 5% CO2, for 7-10 days until confluency was reached. Media was replaced every 2-3 days. Microglia were removed by shaking for 30 minutes at 180 rpm, and the media was changed, then cells were shaken for 2 hours at 220 rpm and the media was changed again. Remaining attached cells were enriched astrocytes. To obtain microglia, media from shaken cells was centrifuged at 500 g for 5 min at 4 C, pellet was resuspended in 0.5% BSA, 2 mM EDTA in 1×PBS, and magnetic sorted using anti-mouse CD11b Microbeads according to the manufacturer's protocol (Miltenyi, #130-049-601).

Primary astrocyte and microglia cytokine stimulation. Cytokine treatment was performed for 18 hours with cytokines diluted in DMEM/F12+GlutaMAX (Life Technologies, #10565042) supplemented with 10% FBS (Life Technologies, #10438026) and 1% penicillin/streptomycin (Life Technologies, #15140122). The following recombinant cytokines were used to stimulate microglia: 50 ng/mL TNF (R&D Systems, #410-MT-010), 100 ng/mL IL-1β (R&D Systems, #401-ML-005). Recombinant mouse semaphorin 4D (24-711) extracellular domain (VWR, #75791- 390), which possesses agonistic activity (S18-S20) was used to stimulate astrocytes at 1 μg/mL.

RNA isolation from cultured mouse astrocytes and microglia. Primary astrocytes were lysed in Buffer RLT (Qiagen) and RNA was isolated from cultured astrocytes using the Qiagen RNeasy Mini kit (Qiagen, #74106). cDNA was transcribed using the high-capacity cDNA Reverse Transcription Kit (Life Technologies, #4368813). Gene expression was then measured by qPCR using Taqman Fast Universal PCR Master Mix (Life Technologies, #4367846). Taqman probes used in this study are: Gapdh (Mm99999915_g1), Nos2 (Mm00440502_m1), Il1b (Mm00434228_m1), and Sema4d (Mm00443147_m1). qPCR data were analyzed by the ddCt method by normalizing the expression of each gene for each replicate to Gapdh and then to the control group.

Bulk RNA-seq. Bulk RNA isolated from flow cytometry sorted cells was used as input with the kit (NEB, #E6420) according to the manufacturer's protocol. Reverse transcription was performed according to the SMART protocol using a template switching oligo. Then, cDNA was amplified and cleaned using Ampure XP beads (Beckman-Coulter, #A63881) and quantified using a Bioanalyzer DNA HS assay (Agilent, #50674626). Libraries were then fragmented, end-repaired, and ligated to Illumina compatible adaptors followed by sample barcoding using NEBNext Multiplex Oligos for Illumina (#E7335S, #E7500S). Samples were selected again using Ampure XP beads and quantified using a Bioanalyzer. Libraries were quantified using a Kapa Library Quantification Kit (Kapa Biosystems, #KK4824) and run on an Illumina NextSeq550.

Modeling of barcode labeling and network capture. The fraction (F) of uniquely labeled cells based on barcode library diversity (N) and number of cells seeded (k) were predicted using the “birthday problem” equation:

$$F = {( 1 - \frac{1}{N} )}^{( k - 1 )}$$

Curves shown in FIG. 1E were then generated based on the fixed library sizes of k=100, 1000, 10000, and 100000. In a separate analysis in FIG. 1F, to estimate the network ratio captured (R) using inDrop we assumed:

$$T = \frac{S \cdot C}{3000} * t$$

where T is the total time required to process all labeled cells in a RABID-seq experiment, given the number of seeded cells (S), the number of connections per cell (C), an inDrop batch size of 3000 cells, and the time required to process each batch (t), which we approximated as 0.5 hours.

We assumed 12 hours is the maximum time that could be spent processing all inDrop samples in a day and 0.6 is the maximum fraction of input cells captured by inDrop. Thus, the ratio of the total seeded network (R) was estimated using the equations:

$$R ( T ) = \{ \begin{matrix}0.6 , & T \leq 12 \\ \frac{12}{T} * 0.6 , & T > 12\end{matrix}$$

R was then calculated for 500, 1000, 2000, 3000, 4000, 5000, 10000, 15000, and 20000 seeded cells and 1-20, 40, 60, 80, 100, 150, and 200 connections per cell.

Single-cell transcriptome analysis. Sequencing reads were processed using the inDrop pipeline available on Github (github.com/indrops/indrops) (S21). Transcriptome data were aligned and quantified using the mouse reference genome GrCM38. Samples were merged and any batch effects were corrected using the canonical correlation approach (S22) built in the R package Seurat. A graphics-based clustering analysis was conducted and cluster markers were used to identify different cell types with the R package SingleR (S23).

Pooling of samples into mice. Cells from each mouse were split into samples of less than 6,000 cells at the time of cell encapsulation in droplets. Samples were processed separately during library prep and sequencing and recombined at the time of data processing. A mouse and sample ID were added to each cell barcode to avoid barcode collisions. A mouse ID was added to each RabΔG barcode to ensure no cross-mouse connections were assigned during network analysis.

Connectome barcode extraction and analysis. Rabies barcodes were recovered from libraries by extracting the 28 base sequence between known flanking handle sequences as follows. If only a 5′ handle existed, 28 bases downstream were selected. If only the 3′ handles existed, 28 bases upstream were selected. If both handles were present, the entire internal sequence was extracted and confirmed to be 28 bases long. The structure of the barcode was checked using regex pattern matching and all sequences that did not conform to the designed sequence (VHDBVHDBATVHDBVHDBATVHDBVHDB) (SEQ ID NO:1) were removed from further analysis. Next, barcodes were error corrected at a Levenshtein distance of one using starcode with ‘—distance 1’ (code available at github.com/guillaume/starcode) (S24). A count matrix of RabΔG barcodes was generated by UMI counting to create a table of RabΔG barcodes, UMI counts, and cell barcodes.

Rarefaction analysis. Rarefaction analysis was performed to ensure that sufficient sequencing depth was obtained in RABID-seq datasets. We randomly sampled 1k, 5k, 10k, 100k, and 500k reads from paired-end .fastq files and applied our pipeline for barcode recovery described above. The number of unique RabΔG barcode sequences was determined and plotted as a function of the number of input reads.

Network analysis. Analysis of RABID-seq datasets was performed in R using the igraph package (version 1.2.5) in R (S25). Connectome data was used to generate graph objects with vertexes representing cells and edges representing shared rabies barcodes. For each vertex, edges were removed if they contained counts less than half of the maximum count in a given vertex. The strength of the edges was calculated as the average of the UMI counts for each shared rabies barcode. Vertexes were removed if they did not contain corresponding transcriptome information (i.e., if a cell barcode was not found in the scRNA-seq dataset). Vertexes were then assigned metadata corresponding to their cell type based on cell calling done with scRNA-seq data (described above). Each vertex, which represented an individual cell, contained full transcriptome information (gene name and normalized counts). Summaries of connections by cell type in the form of chord diagrams were performed using the chorddiag package in R. Astrocyte-centered networks were visualized using the edge weights described above and the Fruchterman-Reingold layout. We centered the networks on astrocytes by removing connections between other cell types. This is justified because we genetically targeted Gfap-expressing cells with the EnvA-TVA system. Thus, astrocytes were the initial progenitors of virus, and shared RabAGbarcodes between other cells are result of their shared connections to an astrocyte. Analysis of the interactions between cells was performed by generating subnetworks based on the characteristics (cell type, gene expression, inflammation score, etc.) of vertices. First, we found 2 sets of vertices, for example astrocytes expressing Il10ra and T cells expressing Il10. Next we extracted the edges between these sets. Lastly, we generated subgraphs from edge lists to create subnetworks that contained cells with only the desired properties (astrocytes expressing Il10ra connected to T cells expressing Il10). We inferred possible ligand receptor interactions using CellPhoneDB (S26) on cells extracted from subnetworks.

Pathway analysis. GSEA pre-ranked analyses (S27) were used to generate enrichment of gene sets in subsets of cells extracted from the network analysis. Genes were ranked based on log(FoldChange) differences in gene expression between two cell populations, for example astrocytes connected to microglia in EAE vs. astrocytes connected to microglia in naïve. GSEA analysis used the KEGG/Reactome/Biocarta (c2.cp.a11), Gene ontology (c5.cp.a11), and Hallmark (h.all) gene sets from Molecular Signatures Database v7.1. Overrepresented transcriptional motifs and pathways were found using Ingenuity Pathway Analysis (IPA) software (Qiagen) and ENRICHR (S28, S29) on lists of differentially expressed genes.

Astrocyte pro-inflammatory score. An inflammation score was calculated for each astrocyte based on its gene expression profile. The inflammation score was determined by first ranking each gene by its scaled counts. The sum of the rank of each gene in gene set M15877 (GO: POSITIVE_REGULATION_OF_INFLAMMATORY_RESPONSE) minus the sum of the rank of each gene in the gene set M13807 (GO: NEGATIVE_REGULATION_OF_INFLAMMATORY_RESPONSE) was calculated. This inflammation score was used to bin astrocytes; those in the bottom 10% and top 90% were extracted from the full network and their adjacent vertices were determined. These subnetworks represented cells connected to astrocytes expressing high pro-inflammatory and low pro-inflammatory transcriptional programs. Differential expression on cells in these subnetworks was performed using Seurat, and the corresponding gene lists were analyzed by GSEA pre-ranked analyses as described above.

### Example 1: RABID-seq Detects Astrocyte Cell Interactions in Naïve and EAE Mice

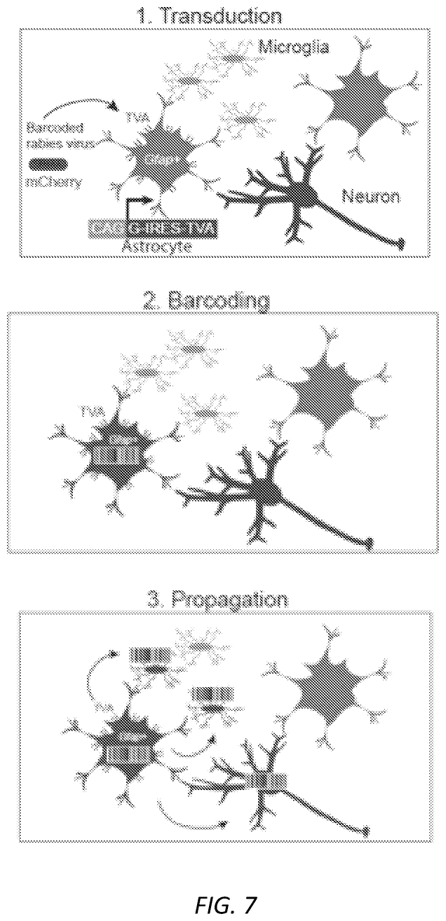

We used transgenic mice expressing the rabies glycoprotein G and the EnvA receptor (TVA) under the control of the Gfap promoter (Gfapp^{TVA/G} mice) (27) to target the initial infection of pseudotyped RabΔG-mCherry-BC virus to astrocytes and limit the subsequent transfer of barcodes (FIGS. 2A, 7). Specifically, the RabΔG-mCherry-BC virus can only initially infect Gfap⁺ astrocytes expressing TVA, which also express the rabies glycoprotein G. New viral particles produced by Gfap⁺ astrocytes incorporate the rabies glycoprotein G into their envelopes, thereby acquiring the ability to infect and barcode neighboring cells. However, since Gfap neighboring cells do not express the rabies glycoprotein G, they cannot further disseminate the virus (28, 29). Using this system, we detected the spread of RabΔG-mCherry-BC by flow cytometry, which peaked 7 days post-transduction. We titered the RabΔG-mCherry-BC virus to seed 1,000 cells in Gfap^{TVA/G} mice upon infection. Next, we transduced with RabΔG-mCherry-BC virus the forebrain of naïve and EAE Gfap^{TVA/G} mice 12 days after disease induction by immunization with MOG₃₅₋₅₅ (FIG. 2B). At 7.5 days post-transduction we sorted mCherry⁺ cells by flow cytometry and analyzed them by scRNA-seq. Cell types were evenly distributed across each sample analyzed by RABID-seq in naïve and EAE mice. We detected RabΔG-mCherry-encoded barcodes in all samples analyzed and confirmed sufficient sequencing depth for all samples. Specifically, we detected an average of 1,000 barcodes per mouse across the 8 mice analyzed, consistent with our target seeding rate, previously set to minimize barcode collisions (FIGS. 1E-1F).

We analyzed by scRNA-seq 32,280 RabΔG-barcoded cells including astrocytes, microglia, monocytes, and T cells (FIG. 2C). Of note, our cell isolation method removed oligodendrocytes and neurons in order to focus on astrocytes, microglia, and infiltrating immune cells. In naïve mice, we detected astrocyte-astrocyte interactions, as well as interactions with microglia and other cells. In EAE mice, astrocyte interaction networks were more diverse (FIG. 2D), and included interactions with peripheral cells recruited to the inflamed CNS such as T cells (1, 2, 14, 30).

We developed an inflammation score based on the activation of the inflammatory response defined by Gene Ontology. We then selected astrocytes displaying the highest (>90^{th} percentile) and lowest (<10^{th} percentile) pro-inflammatory transcriptional phenotypes in EAE that displayed interactions with T cells. Astrocytes displaying the highest pro-inflammatory scores were connected to T cells which exhibited pro-inflammatory phenotypes and high TNFα signaling via NF-κB (136 Astrocytes, 506 T cells, 3796 connections), in agreement with the reported boost of pro-inflammatory astrocyte responses by pro-inflammatory T cells (1). Conversely, T cells connected to astrocytes displaying the lowest pro-inflammatory phenotype (132 astrocytes, 684 T cells, 3,847 connections) showed higher expression of molecules associated with the suppression of inflammation (e.g. Ctla4, Ikzf4, Il2ra, and Il10). Indeed, when we analyzed subnetworks of Il10ra⁺ astrocytes and Il10⁺ T cells, we detected IL2-STAT5 signaling pathways which have been associated with regulatory T cells (31), recapitulating IL-10-driven anti-inflammatory effects of T cells on astrocytes (32, 33). Hence, RABID-seq can be used to simultaneously identify astrocyte cell interactions and the transcriptional features of interacting cells at the single-cell level.

### Example 2: Identification of Sema4D-PlexinB2 Microglia-Astrocyte Signaling by RABID-seq

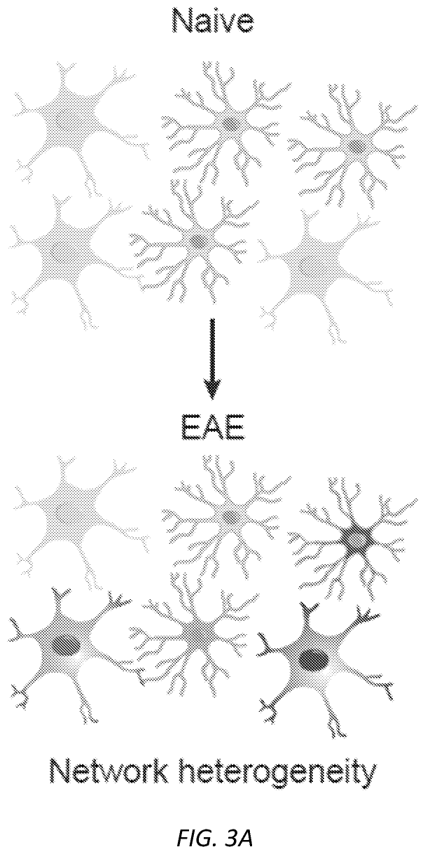

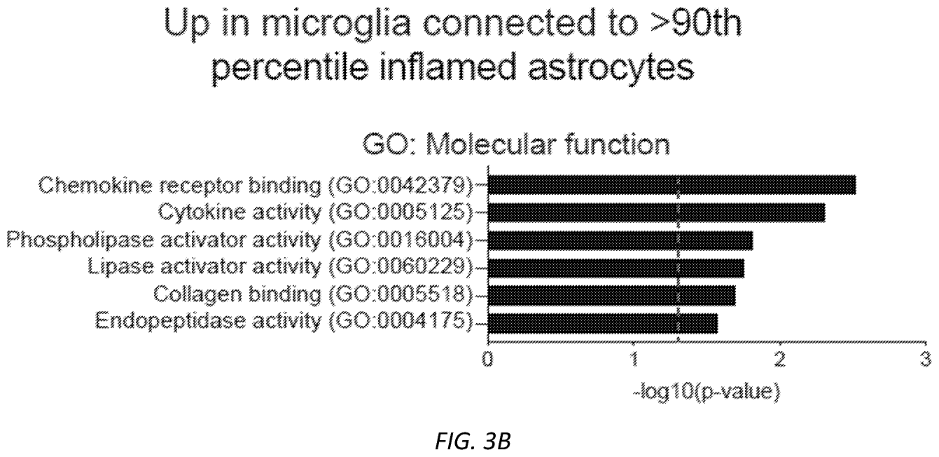

Astrocyte-microglia interactions play important roles during CNS development, homeostasis and disease (8-10). However, we still lack a comprehensive understanding of these interactions and how they shift during inflammation (FIG. 3A). RABID-seq detected the microglial control of astrocytes in EAE mediated by IL-1, TNF, and Clq, in agreement with previous reports (8). Moreover, we detected the activation of pro-inflammatory signatures and chemokine-mediated signaling in microglia connected to astrocytes displaying a high pro-inflammatory phenotype (>90^{th} percentile). Indeed, the analysis of ligand-receptor interactions (34) between these high pro-inflammatory phenotype astrocytes (>90^{th} percentile) and microglia in EAE recapitulated previous reports (10) of increased pro-inflammatory FLT1 signaling and decreased aryl hydrocarbon receptor-driven anti-inflammatory responses in astrocytes triggered by microglia-produced VEGF-B.

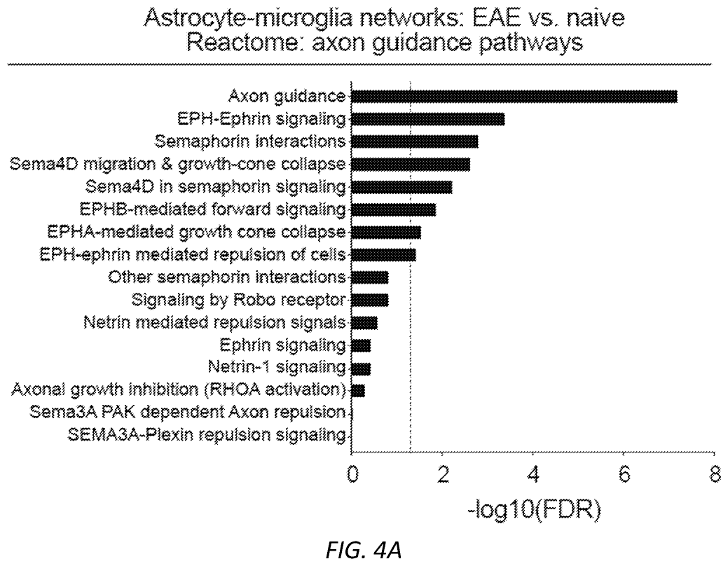

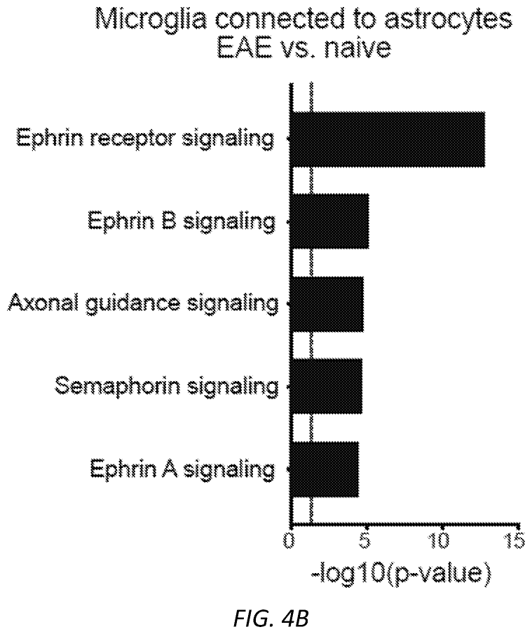

We then used RABID-seq to identify novel mechanisms of astrocyte-microglia communication during EAE, detecting the activation of pathways associated with axon guidance molecules. Axon guidance molecules play important roles during development, but are co-opted by tumors and inflammatory processes in the periphery (35). Thus, we examined axon guidance pathways associated with semaphorin-plexin, EPH-ephrin, netrin, and Slit/Robo signaling in the astrocyte-microglia cell networks that we identified in naive and EAE mice. The analysis of the single cell RABID-seq dataset detected the activation of semaphorin 4D (Sema4D)-PlexinB signaling during EAE (FIGS. 4A-4B), driven by Sema4d expression in microglia and Plxnb2 expression in astrocytes. We also detected the activation of EPH-ephrin B signaling.

Although Sema4D signals through the PlexinB1 and PlexinB2 receptors (35, 36), an analysis of the RABID-seq dataset detected higher Plxnb2 expression in astrocytes in EAE. Hence, to investigate the role of Sema4D-PlexinB2 signaling during EAE, we analyzed the single-cell transcriptional signatures of interacting Plxnb2⁺ astrocytes and Sema4d⁺ microglia detected by RABID-seq. Specifically, we subdivided our RABID-seq data into non-overlapping networks of Plxnb2⁺ or Plxnb2⁻ astrocytes, connected to Sema4d⁺ or Sema4d⁻ microglia. Plxnb2⁺ astrocytes were found to interact preferentially with Sema4d⁺ microglia, exhibiting increased activation of semaphorin-plexin signaling concomitant with increased activation of pro-inflammatory responses. In addition, the analysis of a published scRNA-seq dataset of 48 MS patients and matched controls (1), detected increased interactions between microglial SEMA4D and astrocytic PLXNB2 in MS patients. Collectively, these data suggest that microglia-astrocyte interactions mediated by Sema4D-PlexinB2 promote CNS inflammation during EAE, and potentially, MS.

### Example 3: Microglia-astrocyte Sema4D-PlexinB2 Signaling Promotes CNS Inflammation in EAE

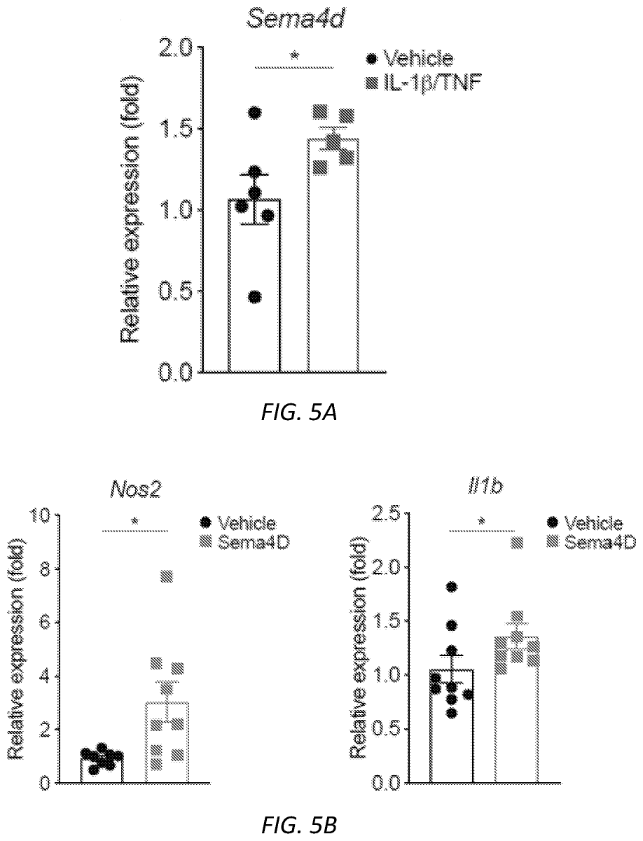

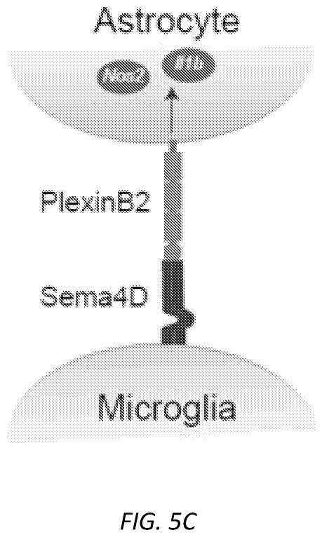

We then investigated the role in EAE pathogenesis of microglia-astrocyte interactions mediated by Sema4D-PlexinB2 signaling. By immunostaining, we detected increased expression of Sema4D in microglia and PlexinB2 in astrocytes during EAE, validating our RABID-seq findings. Moreover, the activation of primary mouse microglia in culture with IL-1β/TNF, pro-inflammatory cytokines known to contribute to the pathogenesis of EAE and MS (1), increased Sema4d expression (FIG. 5A). In addition, the treatment of primary mouse astrocytes with a recombinant Sema4D fragment with plexin agonist activity (37-39) increased the expression of the pro-inflammatory genes Nos2 and Il1b (FIG. 5B), suggesting that Sema4D-triggered plexin signaling boost astrocyte pro-inflammatory responses.

To determine whether microglia-astrocyte interactions mediated by Sema4D-PlexinB2 promote CNS inflammation during EAE, we developed CRISPR/Cas9 lentiviruses to inactivate Sema4d in microglia and Plxnb2 in astrocytes using Itgam- or Gfap-driven Cas9 and targeting sgRNA, respectively. The inactivation of Sema4d in microglia ameliorated EAE. Moreover, astrocytes isolated from Itgam-driven Sema4d inactivation mice showed decreased activation of Nos2 and pro-inflammatory signaling, supporting a role for microglia-expressed Sema4D in promoting astrocyte pathogenic activities. Indeed, Plxnb2 inactivation in astrocytes also resulted in EAE amelioration, concomitant with the downregulation of pro-inflammatory pathways in astrocytes driven primarily by NOS2 and IL-1β.

PlexinB1 and PlexinB2 show redundance in some, but not all, biological systems (40, 41). The analysis of our RABID-seq dataset revealed that PlexinB1 and PlexinB2 are expressed by different astrocyte subpopulations, and that Plxnb2⁺ astrocytes are more abundant than Plxnb1⁺ astrocytes. Thus, we investigated whether Sema4D signaling via PlexinB1 might also contribute to EAE pathogenesis. We found that Gfap-driven Cas9 Plxnb1 inactivation in astrocytes also decreased IL-1β and NOS2 signaling and ameliorated EAE, although to a lesser extent than Plxnb2. Indeed, we also detected increased interactions between microglial SEMA4D and PLXNB1 expressed in astrocytes in MS patients (1), although the detected increase in this interaction in MS was lower than the increase detected for SEIVIA4D-PLXNB2. Taken together, these findings suggest that microglia-astrocyte Sema4D-PlexinB2 (and to a lesser extent Sema4D-PlexinB1) interactions promote CNS inflammation during EAE.

### Example 4: Cell-Cell Interactions in Humans and Non-Human Primates

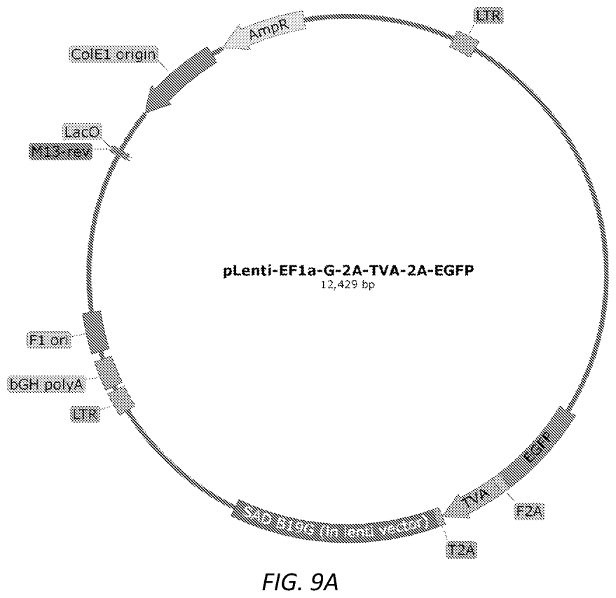

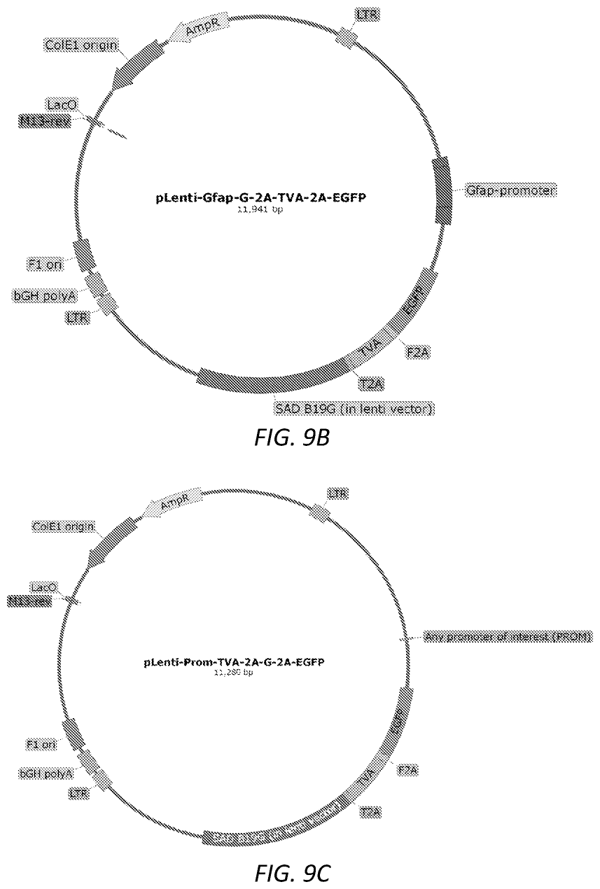

To study cell-cell interaction networks in humans and non-human primates, we developed pLenti-EFla::G-2A-TVA-2A-EGFP (FIG. 9A) and pLenti-Gfap::G-2A-TVA-2A-EGFP (FIG. 9B) lentiviral vectors that target all cells and astrocytes, respectively (derived from the Addgene plasmid 30195, see “Cortical representations of olfactory input by trans-synaptic tracing,” Miyamichi K, Amat F, Moussavi F, Wang C, Wickersham I, Wall NR, Taniguchi H, Tasic B, Huang ZJ, He Z, Callaway EM, Horowitz MA, Luo L. Nature. 2011 Apr 14; 472(7342):191-196. Epub 2010 Dec 22. 10.1038/nature09714 , pBOB-synP-HTB). These vectors are delivered to cultured explant tissue to express the rabies glycoprotein and the TVA receptor for 3 days. Next, we transduce wild-type human and non-human primate tissue with pseudotyped RabDG-BC virus for 3-7 days. Thereafter, we perform a RABID-seq workflow as described herein to isolate mCherry+ cells and perform scRNA-seq.

These plasmids comprise nucleic acid sequences encoding the SAD B19G protein, TVA, and 2A cleavage sites, driven by the EF1a and Gfap promoters for pLenti-EF1a::G-2A-TVA-2A-EGFP and pLenti-Gfap::G-2A-TVA-2A-EGFP, respectively.

TVA nucleotide sequence (SEQ ID NO: 79):
ATGGCGCGGCTGCTGCCCGCGCTGCTGCTGCTGCTGCTGC
CCGGTAACGTGACCGGTAACGGGTCCGGTAACGGTTCTTT
GTCCCGTTGCCCCCCCGGTCAGTTCCGCTGCTCGGAGCCG
CCCGGTGCCCACGGGGAGTGTTACCCGCAGGACTGGCTGT
GCGACGGACACCCCGACTGCGACGACGGGCGGGACGAGTG
GGGCTGCGGGACCAGCGCGATCCCCGCGGTGCCCACGGAC
AACGGCACAGAGGCTCCCACTGCCCCTGCTCCTGGACGTG
CTCTGCCAGCCAGGAATCACGGCCGCATGTGGATGCTGAT
CACTGCAGTGCTCCTGTGCTGCCTGGTAGCTGTGGGTGGT
ATCGCTGCATGGGGGAAGTCCAAAGCAAAAAGCAGGTCTG
ACATCTTCAGTCTTGAAAGCGCATCCAAGGAGCTGCTGGT
GCCTGACAAGAGCCAGGCAGACTTGTTCTCCGGTAGTGGA
TVA amino acid sequence (SEQ ID NO: 80):
MARLLPALLLLLLPGNVTGNGSGNGSLSRCPPGQFRCSEP
PGAHGECYPQDWLCDGHPDCDDGRDEWGCGTSAIPAVPTD
NGTEAPTAPAPGRALPARNHGRMWMLITAVLLCCLVAVGG
IAAWGKSKAKSRSDIFSLESASKELLVPDKSQADLFSGSG

### Example 5: Cell-Cell Interactions in Humans and Non-Human Primates

Further viral vectors were developed to express a tissue specific promoter (e.g., Gfap, Itgam, Ef1a), the avian receptor TVA, the SAD B19 rabies glycoprotein (G), and EGFP, each separated by a 2A element for bicistronic expression (pLenti-Prom-TVA-2A-G-2A-EGFP, FIG. 9C).

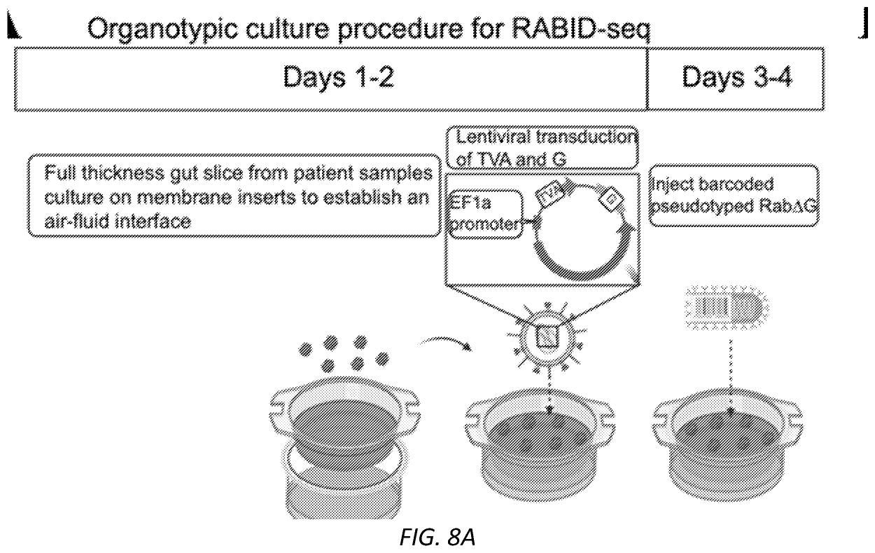

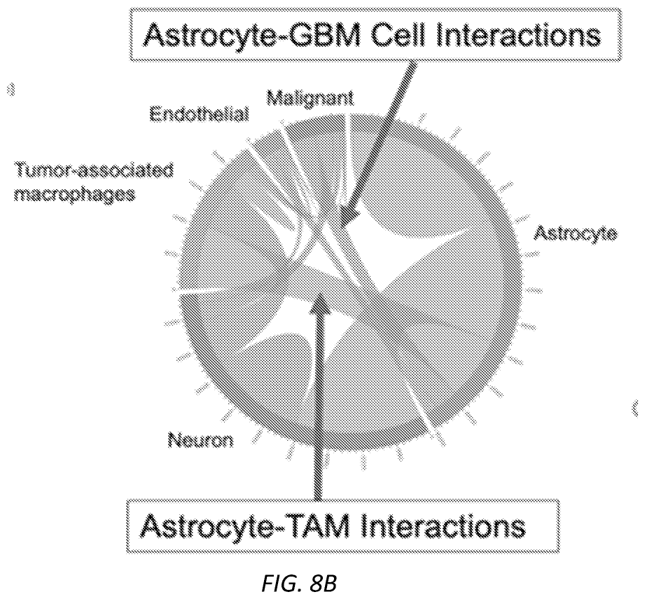

Surgically resected or low post-mortem interval CNS tissue will be sectioned on a vibratome as 350 μm sections. Tissue sections will be cultured in Hibernate-A combined with Polybrene and transduced with pLenti-Prom-TVA-2A-G-2A-EGFP for 5 days (FIG. 8A). Thereafter, cultures are transduced with RabΔG-mCherry-BC for 5 days (FIG. 8A). Following rabies virus transduction, tissue will be dissociated by papain digestion as with mouse studies. Next, fluorescently labeled mCherry+ cells will be sorted by flow cytometry, subjected to scRNA-seq workflows, and barcodes will be isolated from sorted cells analogous to mouse studies.

This approach was applied to human organotypic cultures obtained from glioblastoma samples, identifying novel brain tumor cell interactions with neurons, glia, and immune cells (FIG. 8B).

### References

1. M. A. Wheeler et al., MAFG-driven astrocytes promote CNS inflammation. Nature 578, 593-599 (2020).
2. M. A. Wheeler et al., Environmental Control of Astrocyte Pathogenic Activities in CNS Inflammation. Cell 176, 581-596.e518 (2019).
3. M. V. Sofroniew, Astrocyte barriers to neurotoxic inflammation. Nat Rev Neurosci 16, 249-263 (2015).
4. N. Habib et al., Disease-associated astrocytes in Alzheimer's disease and aging. Nature Neuroscience, 1-6 (2020).
5. M. A. Anderson et al., Astrocyte scar formation aids central nervous system axon regeneration. Nature 532, 195-200 (2016).
6. M. A. Anderson et al., Required growth facilitators propel axon regeneration across complete spinal cord injury. Nature 561, 396-400 (2018).
7. X. Tong et al., Astrocyte Kir4. 1 ion channel deficits contribute to neuronal dysfunction in Huntington's disease model mice. Nature neuroscience 17, 694 (2014).
8. S. A. Liddelow et al., Neurotoxic reactive astrocytes are induced by activated microglia. Nature 541, 481-487 (2017).
9. I. D. Vainchtein et al., Astrocyte-derived interleukin-33 promotes microglial synapse engulfment and neural circuit development. Science 359, 1269-1273 (2018).
10. V. Rothhammer et al., Microglial control of astrocytes in response to microbial metabolites. Nature 557, 724-728 (2018).
11. V. Rothhammer et al., Type I interferons and microbial metabolites of tryptophan modulate astrocyte activity and central nervous system inflammation via the aryl hydrocarbon receptor. Nature Medicine 22, 586-597 (2016).
12. A. Fontana, W. Fierz, H. Wekerle, Astrocytes present myelin basic protein to encephalitogenic T-cell lines. Nature 307, 273-276 (1984).
13. D. Sun, H. Wekerle, Ia-restricted encephalitogenic T lymphocytes mediating EAE lyse autoantigen-presenting astrocytes. Nature 320, 70-72 (1986).
14. G. Locatelli et al., Mononuclear phagocytes locally specify and adapt their phenotype in a multiple sclerosis model. Nat Neurosci 21, 1196-1208 (2018).
15. A. Giladi et al., Dissecting cellular crosstalk by sequencing physically interacting cells. Nat Biotechnol, (2020).
16. G. Pasqual et al., Monitoring T cell-dendritic cell interactions in vivo by intercellular enzymatic labelling. Nature 553, 496-500 (2018).

17. B. M. Turczyk et al., Spatial Sequencing: A Perspective. J Biomol Tech 31, 44-46 (2020).
18. M. Stoeckius et al., Simultaneous epitope and transcriptome measurement in single cells. Nat Methods 14, 865-868 (2017).
19. C. Medaglia et al., Spatial reconstruction of immune niches by combining photoactivatable reporters and scRNA-seq. Science 358, 1622-1626 (2017).
20. N. Hagendorf, K. K. Conzelmann, Pseudotyping of G-Gene-Deficient Rabies Virus. Cold Spring Harb Protoc 2015, pdb prot089417 (2015).
21. C. W. Mount, B. Yalcin, K. Cunliffe-Koehler, S. Sundaresh, M. Monje, Monosynaptic tracing maps brain-wide afferent oligodendrocyte precursor cell connectivity. Elife 8, (2019).
22. E. Motori et al., Inflammation-induced alteration of astrocyte mitochondrial dynamics requires autophagy for mitochondrial network maintenance. Cell Metab 18, 844-859 (2013).
23. J. P. Card et al., Pseudorabies virus infection of the rat central nervous system: ultrastructural characterization of viral replication, transport, and pathogenesis. The Journal of Neuroscience 13, 2515-2539 (1993).
24. I. R. Wickersham et al., Monosynaptic restriction of transsynaptic tracing from single, genetically targeted neurons. Neuron 53, 639-647 (2007).
25. N. R. Wall, I. R. Wickersham, A. Cetin, M. De La Parra, E. M. Callaway, Monosynaptic circuit tracing in vivo through Cre-dependent targeting and complementation of modified rabies virus. Proceedings of the National Academy of Sciences of the United States of America 107, 21848-21853 (2010).
26. I. R. Wickersham et al., Monosynaptic restriction of transsynaptic tracing from single, genetically targeted neurons. Neuron 53, 639-647 (2007).
27. A. D. Garcia, N. B. Doan, T. Imura, T. G. Bush, M. V. Sofroniew, GFAP-expressing progenitors are the principal source of constitutive neurogenesis in adult mouse forebrain. Nat Neurosci 7, 1233-1241 (2004).
28. E. M. Callaway, L. Luo, Monosynaptic Circuit Tracing with Glycoprotein-Deleted Rabies Viruses. J Neurosci 35, 8979-8985 (2015).
29. T. R. Reardon et al., Rabies Virus CVS-N2c(DeltaG) Strain Enhances Retrograde Synaptic Transfer and Neuronal Viability. Neuron 89, 711-724 (2016).
30. Z. Kang et al., Astrocyte-restricted ablation of interleukin-17-induced Act1-mediated signaling ameliorates autoimmune encephalomyelitis. Immunity 32, 414-425 (2010).
31. L. Xie, G. R. Choudhury, A. Winters, S. H. Yang, K. Jin, Cerebral regulatory T cells restrain microglia/macrophage-mediated inflammatory responses via IL-10. Eur J Immunol 45, 180-191 (2015).
32. L. Mayo et al., IL-10-dependent Tr1 cells attenuate astrocyte activation and ameliorate chronic central nervous system inflammation. Brain 139, 1939-1957 (2016).
33. M. Ito et al., Brain regulatory T cells suppress astrogliosis and potentiate neurological recovery. Nature 565, 246-250 (2019).
34. R. Vento-Tormo et al., Single-cell reconstruction of the early maternal-fetal interface in humans. Nature 563, 347-353 (2018).
35. A. Kumanogoh, H. Kikutani, Immunological functions of the neuropilins and plexins as receptors for semaphorins. Nat Rev Immunol 13, 802-814 (2013).
36. L. Tamagnone et al., Plexins are a large family of receptors for transmembrane, secreted, and GPI-anchored semaphorins in vertebrates. Cell 99, 71-80 (1999).
37. A. Elhabazi, S. Delaire, A. Bensussan, L. Boumsell, G. Bismuth, Biological Activity of Soluble CD100. I. The Extracellular Region of CD100 Is Released from the Surface of T Lymphocytes by Regulated Proteolysis. The Journal of Immunology 166, 4341-4347 (2001).
38. B. J. C. Janssen et al., Structural basis of semaphorin—plexin signalling. Nature 467, 1118-1122 (2010).
39. L. Zhu et al., Regulated surface expression and shedding support a dual role for semaphorin 4D in platelet responses to vascular injury. Proc Natl Acad Sci U S A 104, 1621-1626 (2007).
40. P. Fazzari et al., Plexin-B1 plays a redundant role during mouse development and in tumour angiogenesis. BMC Dev Biol 7, 55 (2007).
41. E. Paldy et al., Semaphorin 4C Plexin-B2 signaling in peripheral sensory neurons is pronociceptive in a model of inflammatory pain. Nat Commun 8, 176 (2017).
42. X. Wang et al., Three-dimensional intact-tissue sequencing of single-cell transcriptional states. Science 361, (2018).
43. J. R. Moffitt et al., Molecular, spatial, and functional single-cell profiling of the hypothalamic preoptic region. Science 362, (2018).
44. J. H. Lee et al., Fluorescent in situ sequencing (FISSEQ) of RNA for gene expression profiling in intact cells and tissues. Nat Protoc 10, 442-458 (2015).
45. S. G. Rodrigues et al., Slide-seq: A scalable technology for measuring genome-wide expression at high spatial resolution. Science 363, 1463-1467 (2019).
46. P. L. Stahl et al., Visualization and analysis of gene expression in tissue sections by spatial transcriptomics. Science 353, 78-82 (2016).
47. K. H. Chen, A. N. Boettiger, J. R. Moffitt, S. Wang, X. Zhuang, Spatially resolved, highly multiplexed RNA profiling in single cells. Science 348, aaa6090 (2015).
48. J. H. Lee et al., Highly Multiplexed Subcellular RNA Sequencing in Situ. Science 343, 1360-1363 (2014).
49. A. M. Zador et al., Sequencing the connectome. PLoS Biology 10, e1001411 (2012).
50. I. C. Macaulay et al., G&T-seq: parallel sequencing of single-cell genomes and transcriptomes. Nat Methods 12, 519-522 (2015).
51. J. Cao et al., Joint profiling of chromatin accessibility and gene expression in thousands of single cells. Science 361, 1380-1385 (2018).
52. P. Ayata et al., Epigenetic regulation of brain region-specific microglia clearance activity. Nat Neurosci 21, 1049-1060 (2018).
53. A. C. Wendeln et al., Innate immune memory in the brain shapes neurological disease hallmarks. Nature 556, 332-338 (2018).
54. M. Linnerbauer, M. A. Wheeler, F. J. Quintana, Astrocyte Crosstalk in CNS Inflammation. Neuron, (2020).
55. L. Mayo et al., Regulation of astrocyte activation by glycolipids drives chronic CNS inflammation. Nat Med 20, 1147-1156 (2014).
56. V. Rothhammer et al., Sphingosine 1-phosphate receptor modulation suppresses pathogenic astrocyte activation and chronic progressive CNS inflammation. Proc Natl Acad Sci U S A 114, 2012-2017 (2017).
57. I. D. Vainchtein, A. V. Molofsky, Astrocytes and Microglia: In Sickness and in Health. Trends Neurosci 43, 144-154 (2020).
58. M. Nishide, A. Kumanogoh, The role of semaphorins in immune responses and autoimmune rheumatic diseases. Nat Rev Rheumatol 14, 19-31 (2018).
59. G. Neufeld, O. Kessler, The semaphorins: versatile regulators of tumour progression and tumour angiogenesis. Nat Rev Cancer 8, 632-645 (2008).
60. T. S. Tran, A. L. Kolodkin, R. Bharadwaj, Semaphorin regulation of cellular morphology. Annu Rev Cell Dev Biol 23, 263-292 (2007).
61. R. J. Pasterkamp, Getting neural circuits into shape with semaphorins. Nat Rev Neurosci 13, 605-618 (2012).
62. A. Kumanogoh et al., Requirement for the lymphocyte semaphorin, CD100, in the induction of antigen-specific T cells and the maturation of dendritic cells. J Immunol 169, 1175-1181 (2002).
63. T. Okuno et al., Roles of Sema4D-plexin-B1 interactions in the central nervous system for pathogenesis of experimental autoimmune encephalomyelitis. J Immunol 184, 1499-1506 (2010).
64. T. Worzfeld, S. Offermanns, Semaphorins and plexins as therapeutic targets. Nat Rev Drug Discov 13, 603-621 (2014).
65. C. E. Hagberg et al., Vascular endothelial growth factor B controls endothelial fatty acid uptake. Nature 464, 917-921 (2010).
S1. F. Osakada et al., New rabies virus variants for monitoring and manipulating activity and gene expression in defined neural circuits. Neuron 71, 617-631 (2011).
S2. H. Y. Kueh, A. Champhekar, S. L. Nutt, M. B. Elowitz, E. V. Rothenberg, Positive feedback between PU.1 and the cell cycle controls myeloid differentiation. Science 341, 670-673 (2013).
S3. A. Ghanem, K. K. Conzelmann, G gene-deficient single-round rabies viruses for neuronal circuit analysis. Virus Res 216, 41-54 (2016).
S4. J. G. Doench et al., Optimized sgRNA design to maximize activity and minimize off-target effects of CRISPR-Cas9. Nat Biotechnol 34, 184-191 (2016).
S5. F. Osakada, E. M. Callaway, Design and generation of recombinant rabies virus vectors. Nat Protoc 8, 1583-1601 (2013).
S6. M. A. Wheeler et al., MAFG-driven astrocytes promote CNS inflammation. Nature 578, 593-599 (2020).
S7. M. A. Wheeler et al., Environmental Control of Astrocyte Pathogenic Activities in CNS Inflammation. Cell 176, 581-596.e518 (2019).
S8. L. Mayo et al., Regulation of astrocyte activation by glycolipids drives chronic CNS inflammation. Nat Med 20, 1147-1156 (2014).
S9. V. Rothhammer et al., Type I interferons and microbial metabolites of tryptophan modulate astrocyte activity and central nervous system inflammation via the aryl hydrocarbon receptor. Nature Medicine 22, 586-597 (2016).
S10. V. Rothhammer et al., Microglial control of astrocytes in response to microbial metabolites. Nature 557, 724-728 (2018).
S11. R. Zilionis et al., Single-cell barcoding and sequencing using droplet microfluidics. Nature Methods 12, 44-73 (2016).
S12. J. D. Buenrostro, P. G. Giresi, L. C. Zaba, H. Y. Chang, W. J. Greenleaf, Transposition of native chromatin for fast and sensitive epigenomic profiling of open chromatin, DNA-binding proteins and nucleosome position. Nat Methods 10, 1213-1218 (2013).
S13. J. D. Buenrostro, B. Wu, H. Y. Chang, W. J. Greenleaf, ATAC-seq: A Method for Assaying Chromatin Accessibility Genome-Wide. Curr Protoc Mol Biol 109, 21 29 21- 29 (2015).
S14. N. E. Sanjana, 0. Shalem, F. Zhang, Improved vectors and genome-wide libraries for CRISPR screening. Nat Methods 11, 783-784 (2014).
S15. S. Chen et al., Genome-wide CRISPR screen in a mouse model of tumor growth and metastasis. Cell 160, 1246-1260 (2015).
S16. Y. Lee, A. Messing, M. Su, M. Brenner, GFAP promoter elements required for region-specific and astrocyte-specific expression. Glia 56, 481-493 (2008).
S17. T. Wang et al., Gene Essentiality Profiling Reveals Gene Networks and Synthetic Lethal Interactions with Oncogenic Ras. Cell 168, 890-903 e815 (2017).
S18. B. J. C. Janssen et al., Structural basis of semaphorin—plexin signalling. Nature 467, 1118-1122 (2010).
S19. A. Elhabazi, S. Delaire, A. Bensussan, L. Boumsell, G. Bismuth, Biological Activity of Soluble CD100. I. The Extracellular Region of CD100 Is Released from the Surface of T Lymphocytes by Regulated Proteolysis. The Journal of Immunology 166, 4341-4347 (2001).
S20. L. Zhu et al., Regulated surface expression and shedding support a dual role for semaphorin 4D in platelet responses to vascular injury. Proc Natl Acad Sci U S A 104, 1621-1626 (2007).
S21. A. M. Klein et al., Droplet Barcoding for Single-Cell Transcriptomics Applied to Embryonic Stem Cells. Cell 161, 1187-1201 (2015).
S22. T. Stuart et al., Comprehensive Integration of Single-Cell Data. Cell 177, 1888-1902 e1821 (2019).
S23. D. Aran et al., Reference-based analysis of lung single-cell sequencing reveals a transitional profibrotic macrophage. Nat Immunol 20, 163-172 (2019).
S24. E. Zorita, P. Cusco, G. J. Filion, Starcode: sequence clustering based on all-pairs search. Bioinformatics 31, 1913-1919 (2015).
S25. G. C. a. T. Nepusz, The igraph software package for complex network research. InterJournal, Complex Systems 1695, (2006).
S26. M. Efremova, M. Vento-Tormo, S. A. Teichmann, R. Vento-Tormo, CellPhoneDB: inferring cell-cell communication from combined expression of multi-subunit ligand- receptor complexes. Nat Protoc 15, 1484-1506 (2020).
S27. A. Subramanian et al., Gene set enrichment analysis: a knowledge-based approach for interpreting genome-wide expression profiles. Proc Natl Acad Sci U S A 102, 15545- 15550 (2005).
S28. E. Y. Chen et al., Enrichr: interactive and collaborative HTML5 gene list enrichment analysis tool. BMC Bioinformatics 14, 128 (2013).
S29. M. V. Kuleshov et al., Enrichr: a comprehensive gene set enrichment analysis web server 2016 update. Nucleic Acids Res 44, W90-97 (2016).
S30. S. Jakel et al., Altered human oligodendrocyte heterogeneity in multiple sclerosis. Nature 20 566, 543-547 (2019).
S31. L. Schirmer et al., Neuronal vulnerability and multilineage diversity in multiple sclerosis. Nature, (2019).
S32. B. B. Lake et al., Integrative single-cell analysis of transcriptional and epigenetic states in the human adult brain. Nat Biotechnol 36, 70-80 (2018).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A recombinant rabies virus comprising:
a polynucleotide encoding a foreign virus envelope protein; and
a polynucleotide encoding:
a fluorescent protein;
a barcode sequence flanked by a first common sequence and a second common sequence; and
a 3′ poly(A) tail,
wherein the recombinant rabies virus does not encode a functional G protein.

2. The recombinant rabies virus of claim 1, wherein the foreign virus envelope protein is selected from the group consisting of a retrovirus envelope protein, a paramyxovirus envelope protein, an alphavirus envelope protein, an orthomyxovirus envelope protein, a vesiculovirus envelope protein, and combinations thereof.

3. The recombinant rabies virus of claim 2, wherein the foreign virus envelope protein is avian sarcoma leukosis virus (ASLV) envelope EnvA (ASLV EnvA).

4. The recombinant rabies virus of any one of claims 1-3, wherein the fluorescent protein is selected from the group consisting of a green fluorescent protein, a red fluorescent protein, a yellow fluorescent protein, a blue fluorescent protein, a cyan fluorescent protein, an orange fluorescent protein, and combinations thereof.

5. The recombinant rabies virus of claim 4, wherein the fluorescent protein is a red fluorescent protein, optionally mCherry.

6. The recombinant rabies virus of any one of claims claim 1-5, wherein the barcode sequence is about 15 to about 38 nucleotides long.

7. The recombinant rabies virus of any one of claims 1-6, wherein the barcode sequence comprises repeated regions.

8. The recombinant rabies virus of claim 7, wherein the barcode sequence comprises repeats of VHDBVHDB (SEQ ID NO:2).

9. The recombinant rabies virus of claim 8, wherein the barcode sequence comprises three repeats of VHDBVHDB (SEQ ID NO:2).

10. The recombinant rabies virus of claim 8 or claim 9, wherein the repeated region(s) are separated by a spacer.

11. The recombinant rabies virus of claim 10, where the spacer is an AT dinucleotide.

12. The recombinant rabies virus of any one of claims 1-11, wherein the barcode sequence comprises VHDBVHDBATVHDBVHDBATVHDBVHDB (SEQ ID NO:1).

13. A method for identifying cell-cell contacts in a network of living cells, the method comprising:
(i) providing a network of living cells comprising rabies virus-infection-competent cells expressing a receptor for the foreign envelope protein and a functional rabies virus G protein, and rabies virus-infection incompetent cells that cannot be directly infected by the recombinant rabies virus of any one of claims 1-12;
(ii) contacting the network of living cells with the recombinant rabies virus of any one of claims 1-12, and maintaining the network under conditions sufficient for the rabies virus to spread from the rabies virus-infection-competent cells to the rabies virus-infection-incompetent cells; and
(ii) isolating mRNA transcript(s) from cell(s) expressing the fluorescent protein.

14. The method of claim 13 further comprising
(iii) attaching a first adapter comprising a first common sequence to the 5′ end and a second adapter comprising a second common sequence to the 3′ end of the mRNA transcript(s); and
(iv) sequencing the mRNA transcript(s), thereby generating mRNA transcript sequence(s).

15. The method of claim 13 or claim 14, wherein the foreign envelope protein is avian sarcoma leucosis virus (ASLV) envelope EnvA (ASLV EnvA) and the receptor for the foreign envelope protein is ASLV EnvA receptor TVA.

16. The method of any one of claims 13-15, wherein isolating mRNA transcript(s) from cell(s) expressing the fluorescent protein comprises fluorescence-activated cell sorting (FACS).

17. The method of any one of claims 13-15, wherein isolating mRNA transcript(s) from cell(s) expressing the fluorescent protein comprises in situ hybridization, capture or capture of the mRNA transcript(s).

18. The method of any one of claims 13-15, wherein isolating mRNA transcript(s) from cell(s) expressing the fluorescent protein comprises fluorescent in situ hybridization (FISH).

19. The method of any one of claims 13-18, wherein either the first adapter, the second adapter, or both further comprises a cell barcode.

20. The method of any one of claims 13-19, wherein either the first adapter, the second adapter, or both further comprises a unique molecular identifier (UMI).

21. The method of any one of claims 13-20, further comprising analyzing the sequences of the mRNA transcript(s) to trace networks within the target cells by:
(i) identifying rabies virus barcode sequence(s) amongst the mRNA transcript sequence(s); and
(ii) determining which cell(s) of the living network the rabies virus barcode sequence(s) originated from.

22. The method of any one of claims 13-21, wherein the network of living cells is in a living mammal.

23. The method of claim 22, wherein the network of living cells is in the central nervous system of the mammal.

24. The method of any one of claims 13-21, wherein the network of living cells is outside a living mammal.

25. The method of any one of claims 13-24, wherein the network of living cells is a tissue sample from a living mammal. culture.

26. The method of claim 25, wherein the tissue sample is a tumor sample.

27. The method of claim 26, wherein the tumor is a brain tumor.

28. The method of claim 27, wherein the brain tumor is glioblastoma.

29. The method of claims 24, wherein the network of living cells is a mammalian cell

30. The method of any one of claims 25-29, wherein the tissue sample is a xenograft.

31. The method of claim 24, wherein the network of living cells is an organoid.

32. The method of any one of claims 13-31, wherein providing a network of living cells comprises transducing a network of living cells with a vector comprising a nucleic acid sequence encoding a cell- or tissue-specific promoter, a nucleic acid sequence encoding a receptor for the foreign envelope protein, and a nucleic acid sequence encoding a functional rabies virus G protein, wherein the network of living cells comprises multiple cell and/or tissue types, and the cell- or tissue-specific promoter is specific is specific for some, but not all, of the cell and/or tissue types.

33. The method of claim 32 wherein the foreign envelope protein is avian sarcoma leucosis virus (ASLV) envelope EnvA (ASLV EnvA) and the receptor for the foreign envelope protein is the ASLV EnvA receptor TVA.

34. The method of claim 32 or claim 33, wherein the cell- or tissue-specific promoter is a CNS cell specific promoter.

35. The method of any one of claims 32-34, wherein the vector is a lentiviral vector.

36. A composition comprising the recombinant rabies virus of any one of claims 1-12 and a network of living cells comprising rabies virus-infection-competent cells expressing a receptor for the foreign envelope protein and a functional rabies virus G protein, and rabies virus-infection incompetent cells that cannot be directly infected by the recombinant rabies virus of any one of claims 1-12.


SEQUENCE LISTING (85 sequences)

SEQ ID NO: 1 - DNA, 28 bp, Artificial Sequence
  Description of Artificial Sequence Synthetic oligonucleotide
vhdbvhdbatvhdbvhdbatvhdbvhdb

SEQ ID NO: 2 - DNA, 8 bp, Artificial Sequence
  Description of Artificial Sequence Synthetic oligonucleotide
vhdbvhdb

SEQ ID NO: 3 - DNA, 31 bp, Artificial Sequence
  Description of Artificial Sequence Synthetic primer
aaacctgcagggccaccatggtgagcaaggg

SEQ ID NO: 4 - DNA, 44 bp, Artificial Sequence
  Description of Artificial Sequence Synthetic primer
tttccgcggttttttgctagcttacttgtacagctcgtccatgc

SEQ ID NO: 5 - DNA, 100 bp, Artificial Sequence
  Description of Artificial Sequence Synthetic polynucleotide
aagtaagctagagatgtccacgaggtctctgctagcvhdbvhdbatvhdbvhdbatvhdbvhdbggcgcgcccgtacgctgcaggtcgacccgcggtagc

SEQ ID NO: 6 - DNA, 84 bp, Artificial Sequence
  Description of Artificial Sequence Synthetic oligonucleotide
caagcagaagacggcatacgagatgcacgaccgtgactggagttcagacgtgtgctcttccgatctgtcgacctgcagcgtacg

SEQ ID NO: 7 - DNA, 76 bp, Artificial Sequence
  Description of Artificial Sequence Synthetic oligonucleotide
aatgatacggcgaccaccgagatctacactctttccctacacgacgctcttccgatctgatgtccacgaggtctct

SEQ ID NO: 8 - DNA, 77 bp, Artificial Sequence
  Description of Artificial Sequence Synthetic oligonucleotide
aatgatacggcgaccaccgagatctacactctttccctacacgacgctcttccgatctcgatgtccacgaggtctct

SEQ ID NO: 9 - DNA, 78 bp, Artificial Sequence
  Description of Artificial Sequence Synthetic oligonucleotide
aatgatacggcgaccaccgagatctacactctttccctacacgacgctcttccgatctgcgatgtccacgaggtctct

SEQ ID NO: 10 - DNA, 79 bp, Artificial Sequence
  Description of Artificial Sequence Synthetic oligonucleotide
aatgatacggcgaccaccgagatctacactctttccctacacgacgctcttccgatctagcgatgtccacgaggtctct

SEQ ID NO: 11 - DNA, 80 bp, Artificial Sequence
  Description of Artificial Sequence Synthetic oligonucleotide
aatgatacggcgaccaccgagatctacactctttccctacacgacgctcttccgatctcaacgatgtccacgaggtctct

SEQ ID NO: 12 - DNA, 82 bp, Artificial Sequence
  Description of Artificial Sequence Synthetic oligonucleotide
aatgatacggcgaccaccgagatctacactctttccctacacgacgctcttccgatcttgcaccgatgtccacgaggtctct

SEQ ID NO: 13 - DNA, 83 bp, Artificial Sequence
  Description of Artificial Sequence Synthetic oligonucleotide
aatgatacggcgaccaccgagatctacactctttccctacacgacgctcttccgatctacgcaacgatgtccacgaggtctct

SEQ ID NO: 14 - DNA, 84 bp, Artificial Sequence
  Description of Artificial Sequence Synthetic oligonucleotide
aatgatacggcgaccaccgagatctacactctttccctacacgacgctcttccgatctgaagacccgatgtccacgaggtctct

SEQ ID NO: 15 - DNA, 33 bp, Artificial Sequence
  Description of Artificial Sequence Synthetic primer
aagcagtggtatcaacgcagagtacatrgrgrg

SEQ ID NO: 16 - DNA, 15 bp, Artificial Sequence
  Description of Artificial Sequence Synthetic primer
gggtgtcgggtgcag

SEQ ID NO: 17 - DNA, 27 bp, Artificial Sequence
  Description of Artificial Sequence Synthetic primer
aagcagtggtatcaacgcagagtacat

SEQ ID NO: 18 - DNA, 33 bp, Artificial Sequence
  Description of Artificial Sequence Synthetic primer
ctgtctcttatacacatctgacgctgccgacga

SEQ ID NO: 19 - DNA, 20 bp, Artificial Sequence
  Description of Artificial Sequence Synthetic primer
agatgtgtataagagacagt

SEQ ID NO: 20 - DNA, 51 bp, Artificial Sequence
  Description of Artificial Sequence Synthetic primer
  modified_base (30)..(37): This region may encompass one of the following sequences “TAGATCGC” or “CTCTCTAT” or “TATCCTCT” or “AGAGTAGA” or “GTAAGGAG” or “ACTGCATA” or “AAGGAGTA” or “CTAAGCCT” or “GGCTACTC” or “CCTCAGAC” or “TCCTTACG” or “ACGCGTGG” or
  modified_base (30)..(37): CONT. FROM ABOVE “GGAACTCC” or “TGGCCATG” or “GAGAGATT” or “CGCGGTTA” or “GACCGCCA” or “TAAGATGG” or “ATTGACAT” or “AGCCAACT” or “TACTAGGT” or “TCACGGTT” or “TGTAATGA” or “CACGTCAG” or “CTGAATTC” or “CGTACCGG” or “GATGACGG” or
  modified_base (30)..(37): CONT. FROM ABOVE “TATAGACG” or “GTCATTGA” or “GCATCGTT” or “AGGTTGAC” or “TGAAACTG” or “CAATCACA” or “ACATGCAA” or “ATCGCGCC” or “TCGGTTAA”
  See specification as filed for detailed description of substitutions and preferred embodiments
aatgatacggcgaccaccgagatctacacnnnnnnnntcgtcggcagcgtc

SEQ ID NO: 21 - DNA, 39 bp, Artificial Sequence
  Description of Artificial Sequence Synthetic primer
caagcagaagacggcatacgagatgggtgtcgggtgcag

SEQ ID NO: 22 - DNA, 25 bp, Artificial Sequence
  Description of Artificial Sequence Synthetic primer
tacaccatcgtggaacagtacgaac

SEQ ID NO: 23 - DNA, 15 bp, Artificial Sequence
  Description of Artificial Sequence Synthetic primer
gggtgtcgggtgcag

SEQ ID NO: 24 - DNA, 39 bp, Artificial Sequence; identical to SEQ ID NO: 21
  Description of Artificial Sequence Synthetic primer

SEQ ID NO: 25 - DNA, 88 bp, Artificial Sequence
  Description of Artificial Sequence Synthetic primer
  modified_base (30)..(37): This region may encompass one of the following sequences “TAGATCGC” or “CTCTCTAT” or “TATCCTCT” or “AGAGTAGA” or “GTAAGGAG” or “ACTGCATA” or “AAGGAGTA” or “CTAAGCCT” or “GGCTACTC” or “CCTCAGAC” or “TCCTTACG” or “ACGCGTGG” or
  modified_base (30)..(37): CONT. FROM ABOVE “GGAACTCC” or “TGGCCATG” or “GAGAGATT” or “CGCGGTTA” or “GACCGCCA” or “TAAGATGG” or “ATTGACAT” or “AGCCAACT” or “TACTAGGT” or “TCACGGTT” or “TGTAATGA” or “CACGTCAG” or “CTGAATTC” or “CGTACCGG” or “GATGACGG” or
  modified_base (30)..(37): CONT. FROM ABOVE “TATAGACG” or “GTCATTGA” or “GCATCGTT” or “AGGTTGAC” or “TGAAACTG” or “CAATCACA” or “ACATGCAA” or “ATCGCGCC” or “TCGGTTAA”
  See specification as filed for detailed description of substitutions and preferred embodiments
aatgatacggcgaccaccgagatctacacnnnnnnnntcgtcggcagcgtcagatgtgtataagagacaggatgtccacgaggtctct

SEQ ID NO: 26 - DNA, 89 bp, Artificial Sequence
  Description of Artificial Sequence Synthetic primer
  modified_base (30)..(37): This region may encompass one of the following sequences “TAGATCGC” or “CTCTCTAT” or “TATCCTCT” or “AGAGTAGA” or “GTAAGGAG” or “ACTGCATA” or “AAGGAGTA” or “CTAAGCCT” or “GGCTACTC” or “CCTCAGAC” or “TCCTTACG” or “ACGCGTGG” or
  modified_base (30)..(37): CONT. FROM ABOVE “GGAACTCC” or “TGGCCATG” or “GAGAGATT” or “CGCGGTTA” or “GACCGCCA” or “TAAGATGG” or “ATTGACAT” or “AGCCAACT” or “TACTAGGT” or “TCACGGTT” or “TGTAATGA” or “CACGTCAG” or “CTGAATTC” or “CGTACCGG” or “GATGACGG” or
  modified_base (30)..(37): CONT. FROM ABOVE “TATAGACG” or “GTCATTGA” or “GCATCGTT” or “AGGTTGAC” or “TGAAACTG” or “CAATCACA” or “ACATGCAA” or “ATCGCGCC” or “TCGGTTAA”
  See specification as filed for detailed description of substitutions and preferred embodiments
aatgatacggcgaccaccgagatctacacnnnnnnnntcgtcggcagcgtcagatgtgtataagagacagagatgtccacgaggtctct

SEQ ID NO: 27 - DNA, 90 bp, Artificial Sequence
  Description of Artificial Sequence Synthetic primer
  modified_base (30)..(37): This region may encompass one of the following sequences “TAGATCGC” or “CTCTCTAT” or “TATCCTCT” or “AGAGTAGA” or “GTAAGGAG” or “ACTGCATA” or “AAGGAGTA” or “CTAAGCCT” or “GGCTACTC” or “CCTCAGAC” or “TCCTTACG” or “ACGCGTGG” or
  modified_base (30)..(37): CONT. FROM ABOVE “GGAACTCC” or “TGGCCATG” or “GAGAGATT” or “CGCGGTTA” or “GACCGCCA” or “TAAGATGG” or “ATTGACAT” or “AGCCAACT” or “TACTAGGT” or “TCACGGTT” or “TGTAATGA” or “CACGTCAG” or “CTGAATTC” or “CGTACCGG” or “GATGACGG” or
  modified_base (30)..(37): CONT. FROM ABOVE “TATAGACG” or “GTCATTGA” or “GCATCGTT” or “AGGTTGAC” or “TGAAACTG” or “CAATCACA” or “ACATGCAA” or “ATCGCGCC” or “TCGGTTAA”
  See specification as filed for detailed description of substitutions and preferred embodiments
aatgatacggcgaccaccgagatctacacnnnnnnnntcgtcggcagcgtcagatgtgtataagagacagctgatgtccacgaggtctct

SEQ ID NO: 28 - DNA, 91 bp, Artificial Sequence
  Description of Artificial Sequence Synthetic primer
  modified_base (30)..(37): This region may encompass one of the following sequences “TAGATCGC” or “CTCTCTAT” or “TATCCTCT” or “AGAGTAGA” or “GTAAGGAG” or “ACTGCATA” or “AAGGAGTA” or “CTAAGCCT” or “GGCTACTC” or “CCTCAGAC” or “TCCTTACG” or “ACGCGTGG” or
  modified_base (30)..(37): CONT. FROM ABOVE “GGAACTCC” or “TGGCCATG” or “GAGAGATT” or “CGCGGTTA” or “GACCGCCA” or “TAAGATGG” or “ATTGACAT” or “AGCCAACT” or “TACTAGGT” or “TCACGGTT” or “TGTAATGA” or “CACGTCAG” or “CTGAATTC” or “CGTACCGG” or “GATGACGG” or
  modified_base (30)..(37): CONT. FROM ABOVE “TATAGACG” or “GTCATTGA” or “GCATCGTT” or “AGGTTGAC” or “TGAAACTG” or “CAATCACA” or “ACATGCAA” or “ATCGCGCC” or “TCGGTTAA”
  See specification as filed for detailed description of substitutions and preferred embodiments
aatgatacggcgaccaccgagatctacacnnnnnnnntcgtcggcagcgtcagatgtgtataagagacagtccgatgtccacgaggtctct

SEQ ID NO: 29 - DNA, 8 bp, Artificial Sequence
  Description of Artificial Sequence Synthetic oligonucleotide
tagatcgc

SEQ ID NO: 30 - DNA, 8 bp, Artificial Sequence
  Description of Artificial Sequence Synthetic oligonucleotide
ctctctat

SEQ ID NO: 31 - DNA, 8 bp, Artificial Sequence
  Description of Artificial Sequence Synthetic oligonucleotide
tatcctct

SEQ ID NO: 32 - DNA, 8 bp, Artificial Sequence
  Description of Artificial Sequence Synthetic oligonucleotide
agagtaga

SEQ ID NO: 33 - DNA, 8 bp, Artificial Sequence
  Description of Artificial Sequence Synthetic oligonucleotide
gtaaggag

SEQ ID NO: 34 - DNA, 8 bp, Artificial Sequence
  Description of Artificial Sequence Synthetic oligonucleotide
actgcata

SEQ ID NO: 35 - DNA, 8 bp, Artificial Sequence
  Description of Artificial Sequence Synthetic oligonucleotide
aaggagta

SEQ ID NO: 36 - DNA, 8 bp, Artificial Sequence
  Description of Artificial Sequence Synthetic oligonucleotide
ctaagcct

SEQ ID NO: 37 - DNA, 8 bp, Artificial Sequence
  Description of Artificial Sequence Synthetic oligonucleotide
ggctactc

SEQ ID NO: 38 - DNA, 8 bp, Artificial Sequence
  Description of Artificial Sequence Synthetic oligonucleotide
cctcagac

SEQ ID NO: 39 - DNA, 8 bp, Artificial Sequence
  Description of Artificial Sequence Synthetic oligonucleotide
tccttacg

SEQ ID NO: 40 - DNA, 8 bp, Artificial Sequence
  Description of Artificial Sequence Synthetic oligonucleotide
acgcgtgg

SEQ ID NO: 41 - DNA, 8 bp, Artificial Sequence
  Description of Artificial Sequence Synthetic oligonucleotide
ggaactcc

SEQ ID NO: 42 - DNA, 8 bp, Artificial Sequence
  Description of Artificial Sequence Synthetic oligonucleotide
tggccatg

SEQ ID NO: 43 - DNA, 8 bp, Artificial Sequence
  Description of Artificial Sequence Synthetic oligonucleotide
gagagatt

SEQ ID NO: 44 - DNA, 8 bp, Artificial Sequence
  Description of Artificial Sequence Synthetic oligonucleotide
cgcggtta

SEQ ID NO: 45 - DNA, 8 bp, Artificial Sequence
  Description of Artificial Sequence Synthetic oligonucleotide
gaccgcca

SEQ ID NO: 46 - DNA, 8 bp, Artificial Sequence
  Description of Artificial Sequence Synthetic oligonucleotide
taagatgg

SEQ ID NO: 47 - DNA, 8 bp, Artificial Sequence
  Description of Artificial Sequence Synthetic oligonucleotide
attgacat

SEQ ID NO: 48 - DNA, 8 bp, Artificial Sequence
  Description of Artificial Sequence Synthetic oligonucleotide
agccaact

SEQ ID NO: 49 - DNA, 8 bp, Artificial Sequence
  Description of Artificial Sequence Synthetic oligonucleotide
tactaggt

SEQ ID NO: 50 - DNA, 8 bp, Artificial Sequence
  Description of Artificial Sequence Synthetic oligonucleotide
tcacggtt

SEQ ID NO: 51 - DNA, 8 bp, Artificial Sequence
  Description of Artificial Sequence Synthetic oligonucleotide
tgtaatga

SEQ ID NO: 52 - DNA, 8 bp, Artificial Sequence
  Description of Artificial Sequence Synthetic oligonucleotide
cacgtcag

SEQ ID NO: 53 - DNA, 8 bp, Artificial Sequence
  Description of Artificial Sequence Synthetic oligonucleotide
ctgaattc

SEQ ID NO: 54 - DNA, 8 bp, Artificial Sequence
  Description of Artificial Sequence Synthetic oligonucleotide
cgtaccgg

SEQ ID NO: 55 - DNA, 8 bp, Artificial Sequence
  Description of Artificial Sequence Synthetic oligonucleotide
gatgacgg

SEQ ID NO: 56 - DNA, 8 bp, Artificial Sequence
  Description of Artificial Sequence Synthetic oligonucleotide
tatagacg

SEQ ID NO: 57 - DNA, 8 bp, Artificial Sequence
  Description of Artificial Sequence Synthetic oligonucleotide
gtcattga

SEQ ID NO: 58 - DNA, 8 bp, Artificial Sequence
  Description of Artificial Sequence Synthetic oligonucleotide
gcatcgtt

SEQ ID NO: 59 - DNA, 8 bp, Artificial Sequence
  Description of Artificial Sequence Synthetic oligonucleotide
aggttgac

SEQ ID NO: 60 - DNA, 8 bp, Artificial Sequence
  Description of Artificial Sequence Synthetic oligonucleotide
tgaaactg

SEQ ID NO: 61 - DNA, 8 bp, Artificial Sequence
  Description of Artificial Sequence Synthetic oligonucleotide
caatcaca

SEQ ID NO: 62 - DNA, 8 bp, Artificial Sequence
  Description of Artificial Sequence Synthetic oligonucleotide
acatgcaa

SEQ ID NO: 63 - DNA, 8 bp, Artificial Sequence
  Description of Artificial Sequence Synthetic oligonucleotide
atcgcgcc

SEQ ID NO: 64 - DNA, 8 bp, Artificial Sequence
  Description of Artificial Sequence Synthetic oligonucleotide
tcggttaa

SEQ ID NO: 65 - DNA, 23 bp, Artificial Sequence
  Description of Artificial Sequence Synthetic primer
aaaggcgcgccgagggcctattt

SEQ ID NO: 66 - DNA, 33 bp, Artificial Sequence
  Description of Artificial Sequence Synthetic primer
ttttttggtctcccggtgtttcgtcctttccac

SEQ ID NO: 67 - DNA, 62 bp, Artificial Sequence
  Description of Artificial Sequence Synthetic primer
  modified_base (18)..(37): a, c, t, g, unknown or other
  misc_feature (18)..(37): sgRNA substitution site
  See specification as filed for detailed description of substitutions and preferred embodiments
aaaaaaggtctctaccgnnnnnnnnnnnnnnnnnnnngttttagagctagaaatagcaagtt

SEQ ID NO: 68 - DNA, 25 bp, Artificial Sequence
  Description of Artificial Sequence Synthetic primer
gttccctgcaggaaaaaagcaccga

SEQ ID NO: 69 - DNA, 20 bp, Artificial Sequence
  Description of Artificial Sequence Synthetic oligonucleotide
gccgagtagttaaagatgcc

SEQ ID NO: 70 - DNA, 20 bp, Artificial Sequence
  Description of Artificial Sequence Synthetic oligonucleotide
ggggaaggcacagagcacag

SEQ ID NO: 71 - DNA, 20 bp, Artificial Sequence
  Description of Artificial Sequence Synthetic oligonucleotide
ggaggtcaccagccccacgg

SEQ ID NO: 72 - DNA, 20 bp, Artificial Sequence
  Description of Artificial Sequence Synthetic oligonucleotide
gcactaccagagctaactca

SEQ ID NO: 73 - DNA, 11928 bp, Rabies lyssavirus
acgcttaacaaccagatcaaagaaaaaacagacattgtcaattgcaaagcaaaaatgtaacacccctacaatggatgccgacaagattgtattcaaagtcaataatcaggtggtctctttgaagcctgagattatcgtggatcaatatgagtacaagtaccctgccatcaaagatttgaaaaagccctgtataaccctaggaaaggctcccgatttaaataaagcatacaagtcagttttgtcaggcatgagcgccgccaaacttaatcctgacgatgtatgttcctatttggcagcggcaatgcagttttttgaggggacatgtccggaagactggaccagctatggaattgtgattgcacgaaaaggagataagatcaccccaggttctctggtggagataaaacgtactgatgtagaagggaattgggctctgacaggaggcatggaactgacaagagaccccactgtccctgagcatgcgtccttagtcggtcttctcttgagtctgtataggttgagcaaaatatccgggcaaaacactggtaactataagacaaacattgcagacaggatagagcagatttttgagacagccccttttgttaaaatcgtggaacaccatactctaatgacaactcacaaaatgtgtgctaattggagtactataccaaacttcagatttttggccggaacctatgacatgtttttctcccggattgagcatctatattcagcaatcagagtgggcacagttgtcactgcttatgaagactgttcaggactggtatcatttactgggttcataaaacaaatcaatctcaccgctagagaggcaatactatatttcttccacaagaactttgaggaagagataagaagaatgtttgagccagggcaggagacagctgttcctcactcttatttcatccacttccgttcactaggcttgagtgggaaatctccttattcatcaaatgctgttggtcacgtgttcaatctcattcactttgtaggatgctatatgggtcaagtcagatccctaaatgcaacggttattgctgcatgtgctcctcatgaaatgtctgttctagggggctatctgggagaggaattcttcgggaaagggacatttgaaagaagattcttcagagatgagaaagaacttcaagaatacgaggcggctgaactgacaaagactgacgtagcactggcagatgatggaactgtcaactctgacgacgaggactacttttcaggtgaaaccagaagtccggaggctgtttatactcgaatcatgatgaatggaggtcgactaaagagatctcacatacggagatatgtctcagtcagttccaatcatcaagcccgtccaaactcattcgccgagtttctaaacaagacatattcgagtgactcataagaagttgaataacaaaatgccggaaatctacggattgtgtatatccatcatgaaaaaaactaacacccctcctttcgaaccatcccaaacatgagcaagatctttgtcaatcctagtgctattagagccggtctggccgatcttgagatggctgaagaaactgttgatctgatcaatagaaatatcgaagacaatcaggctcatctccaaggggaacccatagaggtggacaatctccctgaggatatggggcgacttcacctggatgatggaaaatcgcccaaccatggtgagatagccaaggtgggagaaggcaagtatcgagaggactttcagatggatgaaggagaggatcctagcttcctgttccagtcatacctggaaaatgttggagtccaaatagtcagacaaatgaggtcaggagagagatttctcaagatatggtcacagaccgtagaagagattatatcctatgtcgcggtcaactttcccaaccctccaggaaagtcttcagaggataaatcaacccagactactggccgagagctcaagaaggagacaacacccactccttctcagagagaaagccaatcatcgaaagccaggatggcggctcaaattgcttctggccctccagcccttgaatggtcggctaccaatgaagaggatgatctatcagtggaggctgagatcgctcaccagattgcagaaagtttctccaaaaaatataagtttccctctcgatcctcagggatactcttgtataattttgagcaattgaaaatgaaccttgatgatatagttaaagaggcaaaaaatgtaccaggtgtgacccgtttagcccatgacgggtccaaactccccctaagatgtgtactgggatgggtcgctttggccaactctaagaaattccagttgttagtcgaatccgacaagctgagtaaaatcatgcaagatgacttgaatcgctatacatcttgctaaccgaacctctcccctcagtccctctagacaataaaatccgagatgtcccaaagtcaacatgaaaaaaacaggcaacaccactgataaaatgaacctcctacgtaagatagtgaaaaaccgcagggacgaggacactcaaaaatcctctcccgcgtcagcccctctggatgacgatgacttgtggcttccaccccctgaatacgtcccgctgaaagaacttacaggcaagaagaacatgaggaacttttgtatcaacggaagggttaaagtgtgtagcccgaatggttactcgttcaggatcctgcggcacattctgaaatcattcgacgagatatattctgggaatcataggatgatcgggttagtcaaagtggttattggactggctttgtcaggatctccagtccctgagggcctgaactgggtatacaaattgaggagaacctttatcttccagtgggctgattccaggggccctcttgaaggggaggagttggaatactctcaggagatcacttgggatgatgatactgagttcgtcggattgcaaataagagtgattgcaaaacagtgtcatatccagggcagagtctggtgtatcaacatgaacccgagagcatgtcaactatggtctgacatgtctcttcagacacaaaggtccgaagaggacaaagattcctctctgcttctagaataatcagattatatcccgcaaatttatcacttgtttacctctggaggagagaacatatgggctcaactccaacccttgggagcaatataacaaaaaacatgttatggtgccattaaaccgctgcatttcatcaaagtcaagttgattacctttacattttgatcctcttggatgtgaaaaaaactattaacatccctcaaaagactcaaggaaagatggttcctcaggctctcctgtttgtaccccttctggtttttccattgtgttttgggaaattccctatttacacgataccagacaagcttggtccctggagtccgattgacatacatcacctcagctgcccaaacaatttggtagtggaggacgaaggatgcaccaacctgtcagggttctcctacatggaacttaaagttggatacatcttagccataaaagtgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccgctggcttcgaactgtaaaaaccaccaaggagtctctcgttatcatatctccaagtgtggcagatttggacccatatgacagatcccttcactcgagggtcttccctagcgggaagtgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtctcgatgcaaacatcaaatgaaaccaaatggtgccctcccgataagttggtgaacctgcacgactttcgctcagacgaaattgagcaccttgttgtagaggagttggtcaggaagagagaggagtgtctggatgcactagagtccatcatgacaaccaagtcagtgagtttcagacgtctcagtcatttaagaaaacttgtccctgggtttggaaaagcatataccatattcaacaagaccttgatggaagccgatgctcactacaagtcagtcagaacttggaatgagatcctcccttcaaaagggtgtttaagagttggggggaggtgtcatcctcatgtgaacggggtgtttttcaatggtataatattaggacctgacggcaatgtcttaatcccagagatgcaatcatccctcctccagcaacatatggagttgttggaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactgtaaggactggccgtcctttcaacgatccaagtcctgaagatcacctccccttggggggttctttttgaaaaacctgggttcaatagtcctccttgaactccatgcaactgggtagattcaagagtcatgagattttcattaatcctctcagttgatcaagcaagatcatgtcgattctcataataggggagatcttctagcagtttcagtgactaacggtactttcattctccaggaactgacaccaacagttgtagacaaaccacggggtgtctcgggtgactctgtgcttgggcacagacaaaggtcatggtgtgttccatgatagcggactcaggatgagttaattgagagaggcagtcttcctcccgtgaaggacataagcagtagctcacaatcatctcgcgtctcagcaaagtgtgcataattataaagtgctgggtcatctaagcttttcagtcgagaaaaaaacattagatcagaagaacaactggcaacacttctcaacctgagacttacttcaagatgctcgatcctggagaggtctatgatgaccctattgacccaatcgagttagaggctgaacccagaggaacccccattgtccccaacatcttgaggaactctgactacaatctcaactctcctttgatagaagatcctgctagactaatgttagaatggttaaaaacagggaatagaccttatcggatgactctaacagacaattgctccaggtctttcagagttttgaaagattatttcaagaaggtagatttgggttctctcaaggtgggcggaatggctgcacagtcaatgatttctctctggttatatggtgcccactctgaatccaacaggagccggagatgtataacagacttggcccatttctattccaagtcgtcccccatagagaagctgttgaatctcacgctaggaaatagagggctgagaatccccccagagggagtgttaagttgccttgagagggttgattatgataatgcatttggaaggtatcttgccaacacgtattcctcttacttgttcttccatgtaatcaccttatacatgaacgccctagactgggatgaagaaaagaccatcctagcattatggaaagatttaacctcagtggacatcgggaaggacttggtaaagttcaaagaccaaatatggggactgctgatcgtgacaaaggactttgtttactcccaaagttccaattgtctttttgacagaaactacacacttatgctaaaagatcttttcttgtctcgcttcaactccttaatggtcttgctctctcccccagagccccgatactcagatgacttgatatctcaactatgccagctgtacattgctggggatcaagtcttgtctatgtgtggaaactccggctatgaagtcatcaaaatattggagccatatgtcgtgaatagtttagtccagagagcagaaaagtttaggcctctcattcattccttgggagactttcctgtatttataaaagacaaggtaagtcaacttgaagagacgttcggtccctgtgcaagaaggttctttagggctctggatcaattcgacaacatacatgacttggtttttgtgtttggctgttacaggcattgggggcacccatatatagattatcgaaagggtctgtcaaaactatatgatcaggttcaccttaaaaaaatgatagataagtcctaccaggagtgcttagcaagcgacctagccaggaggatccttagatggggttttgataagtactccaagtggtatctggattcaagattcctagcccgagaccaccccttgactccttatatcaaaacccaaacatggccacccaaacatattgtagacttggtgggggatacatggcacaagctcccgatcacgcagatctttgagattcctgaatcaatggatccgtcagaaatattggatgacaaatcacattctttcaccagaacgagactagcttcttggctgtcagaaaaccgaggggggcctgttcctagcgaaaaagttattatcacggccctgtctaagccgcctgtcaatccccgagagtttctgaggtctatagacctcggaggattgccagatgaagacttgataattggcctcaagccaaaggaacgggaattgaagattgaaggtcgattctttgctctaatgtcatggaatctaagattgtattttgtcatcactgaaaaactcttggccaactacatcttgccactttttgacgcgctgactatgacagacaacctgaacaaggtgtttaaaaagctgatcgacagggtcaccgggcaagggcttttggactattcaagggtcacatatgcatttcacctggactatgaaaagtggaacaaccatcaaagattagagtcaacagaggatgtattttctgtcctagatcaagtgtttggattgaagagagtgttttctagaacacacgagttttttcaaaaggcctggatctattattcagacagatcagacctcatcgggttacgggaggatcaaatatactgcttagatgcgtccaacggcccaacctgttggaatggccaggatggcgggctagaaggcttacggcagaagggctggagtctagtcagcttattgatgatagatagagaatctcaaatcaggaacacaagaaccaaaatactagctcaaggagacaaccaggttttatgtccgacatacatgttgtcgccagggctatctcaagaggggctcctctatgaattggagagaatatcaaggaatgcactttcgatatacagagccgtcgaggaaggggcatctaagctagggctgatcatcaagaaagaagagaccatgtgtagttatgacttcctcatctatggaaaaacccctttgtttagaggtaacatattggtgcctgagtccaaaagatgggccagagtctcttgcgtctctaatgaccaaatagtcaacctcgccaatataatgtcgacagtgtccaccaatgcgctaacagtggcacaacactctcaatctttgatcaaaccgatgagggattttctgctcatgtcagtacaggcagtctttcactacctgctatttagcccaatcttaaagggaagagtttacaagattctgagcgctgaaggggagagctttctcctagccatgtcaaggataatctatctagatccttctttgggagggatatctggaatgtccctcggaagattccatatacgacagttctcagaccctgtctctgaagggttatccttctggagagagatctggttaagctcccaagagtcctggattcacgcgttgtgtcaagaggctggaaacccagatcttggagagagaacactcgagagcttcactcgccttctagaagatccgaccaccttaaatatcagaggaggggccagtcctaccattctactcaaggatgcaatcagaaaggctttatatgacgaggtggacaaggtggaaaattcagagtttcgagaggcaatcctgttgtccaagacccatagagataattttatactcttcttaatatctgttgagcctctgtttcctcgatttctcagtgagctattcagttcgtcttttttgggaatccccgagtcaatcattggattgatacaaaactcccgaacgataagaaggcagtttagaaagagtctctcaaaaactttagaagaatccttctacaactcagagatccacgggattagtcggatgacccagacacctcagagggttgggggggtgtggccttgctcttcagagagggcagatctacttagggagatctcttggggaagaaaagtggtaggcacgacagttcctcacccttctgagatgttgggattacttcccaagtcctctatttcttgcacttgtggagcaacaggaggaggcaatcctagagtttctgtatcagtactcccgtcctttgatcagtcatttttttcacgaggccccctaaagggatacttgggctcgtccacctctatgtcgacccagctattccatgcatgggaaaaagtcactaatgttcatgtggtgaagagagctctatcgttaaaagaatctataaactggttcattactagagattccaacttggctcaagctctaattaggaacattatgtctctgacaggccctgatttccctctagaggaggcccctgtcttcaaaaggacggggtcagccttgcataggttcaagtctgccagatacagcgaaggagggtattcttctgtctgcccgaacctcctctctcatatttctgttagtacagacaccatgtctgatttgacccaagacgggaagaactacgatttcatgttccagccattgatgctttatgcacagacatggacatcagagctggtacagagagacacaaggctaagagactctacgtttcattggcacctccgatgcaacaggtgtgtgagacccattgacgacgtgaccctggagacctctcagatcttcgagtttccggatgtgtcgaaaagaatatccagaatggtttctggggctgtgcctcacttccagaggcttcccgatatccgtctgagaccaggagattttgaatctctaagcggtagagaaaagtctcaccatatcggatcagctcaggggctcttatactcaatcttagtggcaattcacgactcaggatacaatgatggaaccatcttccctgtcaacatatacggcaaggtttcccctagagactatttgagagggctcgcaaggggagtattgataggatcctcgatttgcttcttgacaagaatgacaaatatcaatattaatagacctcttgaattggtctcaggggtaatctcatatattctcctgaggctagataaccatccctccttgtacataatgctcagagaaccgtctcttagaggagagatattttctatccctcagaaaatccccgccgcttatccaaccactatgaaagaaggcaacagatcaatcttgtgttatctccaacatgtgctacgctatgagcgagagataatcacggcgtctccagagaatgactggctatggatcttttcagactttagaagtgccaaaatgacgtacctatccctcattacttaccagtctcatcttctactccagagggttgagagaaacctatctaagagtatgagagataacctgcgacaattgagttctttgatgaggcaggtgctgggcgggcacggagaagataccttagagtcagacgacaacattcaacgactgctaaaagactctttacgaaggacaagatgggtggatcaagaggtgcgccatgcagctagaaccatgactggagattacagccccaacaagaaggtgtcccgtaaggtaggatgttcagaatgggtctgctctgctcaacaggttgcagtctctacctcagcaaacccggcccctgtctcggagcttgacataagggccctctctaagaggttccagaaccctttgatctcgggcttgagagtggttcagtgggcaaccggtgctcattataagcttaagcctattctagatgatctcaatgttttcccatctctctgccttgtagttggggacgggtcaggggggatatcaagggcagtcctcaacatgtttccagatgccaagcttgtgttcaacagtcttttagaggtgaatgacctgatggcttccggaacacatccactgcctccttcagcaatcatgaggggaggaaatgatatcgtctccagagtgatagatcttgactcaatctgggaaaaaccgtccgacttgagaaacttggcaacctggaaatacttccagtcagtccaaaagcaggtcaacatgtcctatgacctcattatttgcgatgcagaagttactgacattgcatctatcaaccggatcaccctgttaatgtccgattttgcattgtctatagatggaccactctatttggtcttcaaaacttatgggactatgctagtaaatccaaactacaaggctattcaacacctgtcaagagcgttcccctcggtcacagggtttatcacccaagtaacttcgtctttttcatctgagctctacctccgattctccaaacgagggaagtttttcagagatgctgagtacttgacctcttccacccttcgagaaatgagccttgtgttattcaattgtagcagccccaagagtgagatgcagagagctcgttccttgaactatcaggatcttgtgagaggatttcctgaagaaatcatatcaaatccttacaatgagatgatcataactctgattgacagtgatgtagaatcttttctagtccacaagatggttgatgatcttgagttacagaggggaactctgtctaaagtggctatcattatagccatcatgatagttttctccaacagagtcttcaacgtttccaaacccctaactgacccctcgttctatccaccgtctgatcccaaaatcctgaggcacttcaacatatgttgcagtactatgatgtatctatctactgctttaggtgacgtccctagcttcgcaagacttcacgacctgtataacagacctataacttattacttcagaaagcaagtcattcgagggaacgtttatctatcttggagttggtccaacgacacctcagtgttcaaaagggtagcctgtaattctagcctgagtctgtcatctcactggatcaggttgatttacaagatagtgaagactaccagactcgttggcagcatcaaggatctatccagagaagtggaaagacaccttcataggtacaacaggtggatcaccctagaggatatcagatctagatcatccctactagactacagttgcctgtgaaccggatactcctggaagcctgcccatgctaagactcttgtgtgatgtatcttgaaaaaaacaagatcctaaatctgaacctttggttgtttgattgtttttctcatttttgttgtttatttgttaagcgt

SEQ ID NO: 74 - protein, 450 aa, Rabies lyssavirus
MDADKIVFKVNNQVVSLKPEIIVDQYEYKYPAIKDLKKPCITLGKAPDLNKAYKSVLSGMSAAKLNPDDVCSYLAAAMQFFEGTCPEDWTSYGIVIARKGDKITPGSLVEIKRTDVEGNWALTGGMELTRDPTVPEHASLVGLLLSLYRLSKISGQNTGNYKTNIADRIEQIFETAPFVKIVEHHTLMTTHKMCANWSTIPNFRFLAGTYDMFFSRIEHLYSAIRVGTVVTAYEDCSGLVSFTGFIKQINLTAREAILYFFHKNFEEEIRRMFEPGQETAVPHSYFIHFRSLGLSGKSPYSSNAVGHVFNLIHFVGCYMGQVRSLNATVIAACAPHEMSVLGGYLGEEFFGKGTFERRFFRDEKELQEYEAAELTKTDVALADDGTVNSDDEDYFSGETRSPEAVYTRIMMNGGRLKRSHIRRYVSVSSNHQARPNSFAEFLNKTYSSDS

SEQ ID NO: 75 - protein, 297 aa, Rabies lyssavirus
MSKIFVNPSAIRAGLADLEMAEETVDLINRNIEDNQAHLQGEPIEVDNLPEDMGRLHLDDGKSPNHGEIAKVGEGKYREDFQMDEGEDPSFLFQSYLENVGVQIVRQMRSGERFLKIWSQTVEEIISYVAVNFPNPPGKSSEDKSTQTTGRELKKETTPTPSQRESQSSKARMAAQIASGPPALEWSATNEEDDLSVEAEIAHQIAESFSKKYKFPSRSSGILLYNFEQLKMNLDDIVKEAKNVPGVTRLAHDGSKLPLRCVLGWVALANSKKFQLLVESDKLSKIMQDDLNRYTSC

SEQ ID NO: 76 - protein, 202 aa, Rabies lyssavirus
MNLLRKIVKNRRDEDTQKSSPASAPLDDDDLWLPPPEYVPLKELTGKKNMRNFCINGRVKVCSPNGYSFRILRHILKSFDEIYSGNHRMIGLVKVVIGLALSGSPVPEGLNWVYKLRRTFIFQWADSRGPLEGEELEYSQEITWDDDTEFVGLQIRVIAKQCHIQGRVWCINMNPRACQLWSDMSLQTQRSEEDKDSSLLLE

SEQ ID NO: 77 - protein, 523 aa, Rabies lyssavirus
MVPQALLFVPLLVFPLCFGKFPIYTIPDKLGPWSPIDIHHLSCPNLVVEDEGCTNLSGFSYMELKVGYILAIKVNGFTCTGVVTEAETYTNFVGYVTTTFKRKHFRPTPDACRAAYNWKMAGDPRYEESLHNPYPDYRWLRTVKTTKESLVIISPSVADLDPYDRSLHSRVFPSGKCSGVAVSSTYCSTNHDYTIWMPENPRLGMSCDIFTNSRGKRASKGSETCGFVDERGLYKSLKGACKLKLCGVLGLRLMDGTWVSMQTSNETKWCPPDKLVNLHDFRSDEIEHLVVEELVRKREECLDALESIMTTKSVSFRRLSHLRKLVPGFGKAYTIFNKTLMEADAHYKSVRTWNEILPSKGCLRVGGRCHPHVNGVFFNGIILGPDGNVLIPEMQSSLLQQHMELLESSVIPLVHPLADPSTVFKDGDEAEDFVEVHLPDVHNQVSGVDLGLPNWGKYVLLSAGALTALMLIIFLMTCCRRVNRSEPTQHNLRGTGREVSVTPQSGKIISSWESHKSGGETRL

SEQ ID NO: 78 - protein, 2125 aa, Rabies lyssavirus
MLDPGEVYDDPIDPIELEAEPRGTPIVPNILRNSDYNLNSPLIEDPARLMLEWLKTGNRPYRMTLTDNCSRSFRVLKDYFKKVDLGSLKVGGMAAQSMISLWLYGAHSESNRSRRCITDLAHFYSKSSPIEKLLNLTLGNRGLRIPPEGVLSCLERVDYDNAFGRYLANTYSSYLFFHVITLYMNALDWDEEKTILALWKDLTSVDIGKDLVKFKDQIWGLLIVTKDFVYSQSSNCLFDRNYTLMLKDLFLSRFNSLMVLLSPPEPRYSDDLISQLCQLYIAGDQVLSMCGNSGYEVIKILEPYVVNSLVQRAEKFRPLIHSLGDFPVFIKDKVSQLEETFGPCARRFFRALDQFDNIHDLVFVFGCYRHWGHPYIDYRKGLSKLYDQVHLKKMIDKSYQECLASDLARRILRWGFDKYSKWYLDSRFLARDHPLTPYIKTQTWPPKHIVDLVGDTWHKLPITQIFEIPESMDPSEILDDKSHSFTRTRLASWLSENRGGPVPSEKVIITALSKPPVNPREFLRSIDLGGLPDEDLIIGLKPKERELKIEGRFFALMSWNLRLYFVITEKLLANYILPLFDALTMTDNLNKVFKKLIDRVTGQGLLDYSRVTYAFHLDYEKWNNHQRLESTEDVFSVLDQVFGLKRVFSRTHEFFQKAWIYYSDRSDLIGLREDQIYCLDASNGPTCWNGQDGGLEGLRQKGWSLVSLLMIDRESQIRNTRTKILAQGDNQVLCPTYMLSPGLSQEGLLYELERISRNALSIYRAVEEGASKLGLIIKKEETMCSYDFLIYGKTPLFRGNILVPESKRWARVSCVSNDQIVNLANIMSTVSTNALTVAQHSQSLIKPMRDFLLMSVQAVFHYLLFSPILKGRVYKILSAEGESFLLAMSRIIYLDPSLGGISGMSLGRFHIRQFDPVSEGLSFWREIWLSSQESWIHALCQEAGNPDLGERTLESFTRLLEDPTTLNIRGGASPTILLKDAIRKALYDEVDKVENSEFREAILLSKTHRDNFILFLISVEPLFPRFLSELFSSSFLGIPESIIGLIQNSRTIRRQFRKSLSKTLEESFYNSEIHGISRMTQTPQRVGGVWPCSSERADLLREISWGRKVVGTTVPHPSEMLGLLPKSSISCTCGATGGGNPRVSVSVLPSFDQSFFSRGPLKGYLGSSTSMSTQLFHAWEKVTNVHVVKRALSLKESINWFITRDSNLAQALIRNIMSLTGPDFPLEEAPVFKRTGSALHRFKSARYSEGGYSSVCPNLLSHISVSTDTMSDLTQDGKNYDFMFQPLMLYAQTWTSELVQRDTRLRDSTFHWHLRCNRCVRPIDDVTLETSQIFEFPDVSKRISRMVSGAVPHFQRLPDIRLRPGDFESLSGREKSHHIGSAQGLLYSILVAIHDSGYNDGTIFPVNIYGKVSPRDYLRGLARGVLIGSSICFLTRMTNININRPLELVSGVISYILLRLDNHPSLYIMLREPSLRGEIFSIPQKIPAAYPTTMKEGNRSILCYLQHVLRYEREIITASPENDWLWIFSDFRSAKMTYLSLITYQSHLLLQRVERNLSKSMRDNLRQLSSLMRQVLGGHGEDTLESDDNIQRLLKDSLRRTRWVDQEVRHAARTMTGDYSPNKKVSRKVGCSEWVCSAQQVAVSTSANPAPVSELDIRALSKRFQNPLISGLRVVQWATGAHYKLKPILDDLNVFPSLCLVVGDGSGGISRAVLNMFPDAKLVFNSLLEVNDLMASGTHPLPPSAIMRGGNDIVSRVIDLDSIWEKPSDLRNLATWKYFQSVQKQVNMSYDLIICDAEVTDIASINRITLLMSDFALSIDGPLYLVFKTYGTMLVNPNYKAIQHLSRAFPSVTGFITQVTSSFSSELYLRFSKRGKFFRDAEYLTSSTLREMSLVLFNCSPKSEMQRARSLNYQDLVRGFPEEIISNPYNEMIITLIDSDVESFLVHKMVDDLELQRGTLSKVAIIIAIMIVFSNRVFNVSKPLTDPSFYPPSDPKILRHFNICCSTMMYLSTALGDVPSFARLHDLYNRPITYYFRKQVIRGNVYLSWSWSNDTSVFKRVACNSSLSLSSHWIRLIYKIVKTTRLVGSIKDLSREVERHLHRYNRWITLEDIRSRSSLLDYSCL

SEQ ID NO: 79 - DNA, 480 bp, Artificial Sequence
  Description of Artificial Sequence Synthetic polynucleotide
atggcgcggctgctgcccgcgctgctgctgctgctgctgcccggtaacgtgaccggtaacgggtccggtaacggttctttgtcccgttgcccccccggtcagttccgctgctcggagccgcccggtgcccacggggagtgttacccgcaggactggctgtgcgacggacaccccgactgcgacgacgggcgggacgagtggggctgcgggaccagcgcgatccccgcggtgcccacggacaacggcacagaggctcccactgcccctgctcctggacgtgctctgccagccaggaatcacggccgcatgtggatgctgatcactgcagtgctcctgtgctgcctggtagctgtgggtggtatcgctgcatgggggaagtccaaagcaaaaagcaggtctgacatcttcagtcttgaaagcgcatccaaggagctgctggtgcctgacaagagccaggcagacttgttctccggtagtgga

SEQ ID NO: 80 - protein, 160 aa, Artificial Sequence
  Description of Artificial Sequence Synthetic polypeptide
MARLLPALLLLLLPGNVTGNGSGNGSLSRCPPGQFRCSEPPGAHGECYPQDWLCDGHPDCDDGRDEWGCGTSAIPAVPTDNGTEAPTAPAPGRALPARNHGRMWMLITAVLLCCLVAVGGIAAWGKSKAKSRSDIFSLESASKELLVPDKSQADLFSGSG

SEQ ID NO: 81 - DNA, 833 bp, Artificial Sequence
  Description of Artificial Sequence Synthetic polynucleotide
gccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaataagctagagatgtccacgaggtctctgctagcvhdbvhdbatvhdbvhdbatvhdbvhdbggcgcgcccgtacgctgcaggtcgacccgcggtagcttttcagtcgagaaaaaaa

SEQ ID NO: 82 - DNA, 20 bp, Artificial Sequence
  Description of Artificial Sequence Synthetic primer
tttttttttttttttttttt

SEQ ID NO: 83 - DNA, 10 bp, Artificial Sequence
  Description of Artificial Sequence Synthetic oligonucleotide
vhdbvhdbat

SEQ ID NO: 84 - DNA, 10 bp, Artificial Sequence
  Description of Artificial Sequence Synthetic oligonucleotide
vhdbthdbat

SEQ ID NO: 85 - DNA, 11 bp, Artificial Sequence
  Description of Artificial Sequence Synthetic oligonucleotide
vhdbvhdbbga